SEQUENCE LISTING (283 sequences)

SEQ ID NO: 1 - protein, 9 aa, synthetic construct
FAFSTYWMH

SEQ ID NO: 2 - protein, 17 aa, synthetic construct
RINGDGSGTNYADSVKG

SEQ ID NO: 3 - protein, 16 aa, synthetic construct
TRTAGRGAYDFGRFDP

SEQ ID NO: 4 - protein, 16 aa, synthetic construct
RSSQSLLHSNGYNYLD

SEQ ID NO: 5 - protein, 7 aa, synthetic construct
LGSNRAS

SEQ ID NO: 6 - protein, 9 aa, synthetic construct
MQALQIPIT

SEQ ID NO: 7 - protein, 123 aa, synthetic construct
EVQLVESGGGVVQPGGSLRLSCAASGFAFSTYWMHWVRQAPGKGLVWVSRINGDGSGTNYADSVKGRFTISRDNAKSTLYLQMNSLRTEDTAVYYCTRTAGRGAYDFGRFDPWGQGTLVTVSS

SEQ ID NO: 8 - protein, 112 aa, synthetic construct
DIVLTQTPLSLPVTPGEPASISCRSSQSLLHSNGYNYLDWYLQKAGQSPQLLVFLGSNRASGVPDRFSGSASGTDFTLKISRVEAEDVGVYYCMQALQIPITFGQGTRLEIK

SEQ ID NO: 9 - protein, 9 aa, synthetic construct
FTVSSNYMS

SEQ ID NO: 10 - protein, 16 aa, synthetic construct
IIYSGGSTYYADSVKG

SEQ ID NO: 11 - protein, 14 aa, synthetic construct
ARAPSGEYGDPIYY

SEQ ID NO: 12 - protein, 12 aa, synthetic construct
TLSSGHSTYTIA

SEQ ID NO: 13 - protein, 11 aa, synthetic construct
LNSDGSHSKGD

SEQ ID NO: 14 - protein, 10 aa, synthetic construct
QTWGTGNHWV

SEQ ID NO: 15 - protein, 120 aa, synthetic construct
EVQLVESGGGLIQPGGSLRLSCAASGFTVSSNYMSWVRQAPGKGLEWVSIIYSGGSTYYADSVKGRFTISRDNSKNTLYLQMNSLRAEDTAVYYCARAPSGEYGDPIYYWGQGTLVTVSS

SEQ ID NO: 16 - protein, 112 aa, synthetic construct
QSVLTQPPSASASLGASVKLTCTLSSGHSTYTIAWHQQQPEKGPRYLMKLNSDGSHSKGDGIPDRFSGSSSGAERYLTISSLQSEDEADYYCQTWGTGNHWVFGGGTKLTVL

SEQ ID NO: 17 - protein, 11 aa, synthetic construct
GSINSRSYYWG

SEQ ID NO: 18 - protein, 16 aa, synthetic construct
SIYYSGSTYYNPSLKS

SEQ ID NO: 19 - protein, 15 aa, synthetic construct
ARLEQWELPGGWFDP

SEQ ID NO: 20 - protein, 13 aa, synthetic construct
TRSSGSIASNYVQ

SEQ ID NO: 21 - protein, 7 aa, synthetic construct
EDNQRPS

SEQ ID NO: 22 - protein, 9 aa, synthetic construct
QSSDSSNVV

SEQ ID NO: 23 - protein, 123 aa, synthetic construct
EVQLLESGPGLVKPSETLSLTCTVSGGSINSRSYYWGWIRQPPGKGLEWIGSIYYSGSTYYNPSLKSRVTISVDTSKNQFSLKLSSVIAADTAVYFCARLEQWELPGGWFDPWGQGTLVTVSS

SEQ ID NO: 24 - protein, 110 aa, synthetic construct
NFMLTQPHSVSESPGKTVTISCTRSSGSIASNYVQWYQQRPGSAPTTVIYEDNQRPSGVPDRFSGSIDISSNSASLTISGLKTEDEADYSCQSSDSSNVVFGGGTKLTVL

SEQ ID NO: 25 - protein, 9 aa, synthetic construct
FTVSSNYMN

SEQ ID NO: 26 - protein, 16 aa, synthetic construct
VIYSGGSTYYADSVKG

SEQ ID NO: 27 - protein, 10 aa, synthetic construct
AREVYSYFDY

SEQ ID NO: 28 - protein, 11 aa, synthetic construct
RASQGISSYLA

SEQ ID NO: 29 - protein, 7 aa, synthetic construct
AASTLQS

SEQ ID NO: 30 - protein, 10 aa, synthetic construct
QQLNSYPPHT

SEQ ID NO: 31 - protein, 116 aa, synthetic construct
EVQLVESGGGLIQPGGSLTLSCAASGFTVSSNYMNWVRQAPGKGLEWVSVIYSGGSTYYADSVKGRFTISRDNSKNTLYLQMNSLRAEDTAVYYCAREVYSYFDYWGQGTLVTVSS

SEQ ID NO: 32 - protein, 108 aa, synthetic construct
DIQLTQSPSFLSASVGDRVTITCRASQGISSYLAWYQQKPGKAPKLLIYAASTLQSGVPSRFSGSGSGTEFTLTISRLQPEDLATYYCQQLNSYPPHTFGQGAKLEIK

SEQ ID NO: 33 - protein, 9 aa, synthetic construct
FIVSRNYMS

SEQ ID NO: 34 - protein, 16 aa, synthetic construct
VIYSGGNTYYADSVKG

SEQ ID NO: 35 - protein, 12 aa, synthetic construct
ARDLRGPGCFDF

SEQ ID NO: 36 - protein, 11 aa, synthetic construct
QASQDISNFLN

SEQ ID NO: 37 - protein, 7 aa, synthetic construct
DASNLET

SEQ ID NO: 38 - protein, 9 aa, synthetic construct
QQYDNLRVT

SEQ ID NO: 39 - protein, 118 aa, synthetic construct
QVQLVESGGGLIQPGESLRLSCAASEFIVSRNYMSWVRQAPGKGLEWVSVIYSGGNTYYADSVKGRFTISRDNSKNTLYLQMNSLRAEDTAVYYCARDLRGPGCFDFWGQGTTVTVSS

SEQ ID NO: 40 - protein, 107 aa, synthetic construct
DIQVTQSPSSLSASVGDRVTITCQASQDISNFLNWYQQKPGKAPKLLIYDASNLETGVPSRFSASGSGTEFTFTISSLQPEDIATYYCQQYDNLRVTFGGGTKLEIK

SEQ ID NO: 41 - protein, 9 aa, synthetic construct
FTFSNFAMH

SEQ ID NO: 42 - protein, 17 aa, synthetic construct
VILYDGINKYYADSVKG

SEQ ID NO: 43 - protein, 20 aa, synthetic construct
ARAQNYYDRSGTLQLDAFDI

SEQ ID NO: 44 - protein, 11 aa, synthetic construct
RASQGIRNDLG

SEQ ID NO: 45 - protein, 7 aa, synthetic construct
AASSLQS

SEQ ID NO: 46 - protein, 9 aa, synthetic construct
LQDYNYPLT

SEQ ID NO: 47 - protein, 127 aa, synthetic construct
QVQLVQSGGGVVQPGRSLRLSCAASGFTFSNFAMHWVRQAPGKGLEWVAVILYDGINKYYADSVKGRFTISRGNSKNTLSLQMNSLRAEDTAVYYCARAQNYYDRSGTLQLDAFDIWGQGTMVTVSS

SEQ ID NO: 48 - protein, 107 aa, synthetic construct
DIVMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIK

SEQ ID NO: 49 - protein, 9 aa, synthetic construct
YTFTFYYIH

SEQ ID NO: 50 - protein, 17 aa, synthetic construct
WISPISGATNYAQTFQG

SEQ ID NO: 51 - protein, 14 aa, synthetic construct
AREKYYDSSGSSDY

SEQ ID NO: 52 - protein, 13 aa, synthetic construct
SGSSSNIGNNYVS

SEQ ID NO: 53 - protein, 7 aa, synthetic construct
ENNKRPS

SEQ ID NO: 54 - protein, 12 aa, synthetic construct
GTWESSLSAPRV

SEQ ID NO: 55 - protein, 121 aa, synthetic construct
EVQLVESGAEVRKPGASVKVSCKASGYTFTFYYIHWVRQAPGQGLEWMGWISPISGATNYAQTFQGRVTMTRDTSITTAYMELSSLTSDDTAVYYCAREKYYDSSGSSDYWGQGTLVTVSS

SEQ ID NO: 56 - protein, 111 aa, synthetic construct
QPVLTQPPSVSAAPGQNVTIFCSGSSSNIGNNYVSWYQQLPGPAPKLLIYENNKRPSGIPDRFSGSKSGTSATLGITGLQTGDEADYYCGTWESSLSAPRVFGSGTKLTVL

SEQ ID NO: 57 - protein, 9 aa, synthetic construct
FTFSSYAMS

SEQ ID NO: 58 - protein, 17 aa, synthetic construct
AISGSGNSTSYADSVKG

SEQ ID NO: 59 - protein, 16 aa, synthetic construct
AKGLGTRSFNWGAFDY

SEQ ID NO: 60 - protein, 11 aa, synthetic construct
RASQGISYSLA

SEQ ID NO: 61 - protein, 7 aa, synthetic construct
AASNLES

SEQ ID NO: 62 - protein, 9 aa, synthetic construct
QQYYSTPWT

SEQ ID NO: 63 - protein, 123 aa, synthetic construct
EVQLLESGGGLVQPGGSLRLSCAASGFTFSSYAMSWVRQAPGKGLEWVSAISGSGNSTSYADSVKGRFTLSRDNSKNTLYLQMNSLRAEDTAIYYCAKGLGTRSFNWGAFDYWGQGTLVTVSS

SEQ ID NO: 64 - protein, 107 aa, synthetic construct
DIRVTQSPSSLSASVGDRVTITCRASQGISYSLAWYQQKPGKAPKLLLYAASNLESGVPSRFSGSGSGTDYTLTVSSLQPEDFATYYCQQYYSTPWTFGQGTKVEIK

SEQ ID NO: 65 - protein, 9 aa, synthetic construct
FTFADYAMS

SEQ ID NO: 66 - protein, 19 aa, synthetic construct
FIRSKPYGGTTEYAASVKG

SEQ ID NO: 67 - protein, 22 aa, synthetic construct
SRDPWYCSGGDCYAVTGSWFDP

SEQ ID NO: 68 - protein, 11 aa, synthetic construct
QASQDIKKYLN

SEQ ID NO: 69 - protein, 7 aa, synthetic construct
DVSNLET

SEQ ID NO: 70 - protein, 9 aa, synthetic construct
QQYDNLPQT

SEQ ID NO: 71 - protein, 131 aa, synthetic construct
EVQLVESGGGLVKPGRSLRLSCAASGFTFADYAMSWFRQAPGKGLEWVGFIRSKPYGGTTEYAASVKGRFTISRDDSKSIAYLQMNSLKTEDTAVYYCSRDPWYCSGGDCYAVTGSWFDPWGQGTLVTVSS

SEQ ID NO: 72 - protein, 107 aa, synthetic construct
DIVLTQSPSSLSASVGDRVTITCQASQDIKKYLNWYQQKPGKAPKLLIYDVSNLETGVPSRFSGSGSGTDFTFTISSLQPEDVAIYYCQQYDNLPQTFGQGTKVDIK

SEQ ID NO: 73 - protein, 9 aa, synthetic construct
FTFSSYGMH

SEQ ID NO: 74 - protein, 17 aa, synthetic construct
VISYDGSNKYYTDSVKG

SEQ ID NO: 75 - protein, 27 aa, synthetic construct
AKDLPREVYYDILTGYDNPYYYYGMDV

SEQ ID NO: 76 - protein, 12 aa, synthetic construct
RASQSVSSSYLA

SEQ ID NO: 77 - protein, 7 aa, synthetic construct
GASTRAT

SEQ ID NO: 78 - protein, 10 aa, synthetic construct
QQYGSSPRWT

SEQ ID NO: 79 - protein, 134 aa, synthetic construct
QVQLVQSGGGVVQPGRSLRLSCAASGFTFSSYGMHWVRQAPGKGLEWVAVISYDGSNKYYTDSVKGRFTISRDNSKNTLYLQMNSLRAEDTAVYYCAKDLPREVYYDILTGYDNPYYYYGMDVWGQGTTVTVSS

SEQ ID NO: 80 - protein, 109 aa, synthetic construct
EIVMTQSPGTLSLSPGERATLSCRASQSVSSSYLAWYQQKPGQAPRLLIYGASTRATGIPDRFSGSGSGTDFTLTISRLEPEDFAVYYCQQYGSSPRWTFGQGTKVEIK

SEQ ID NO: 81 - protein, 9 aa, synthetic construct
GTFSRYAIS

SEQ ID NO: 82 - protein, 17 aa, synthetic construct
GIIPVFGIVHYAQKFQG

SEQ ID NO: 83 - protein, 18 aa, synthetic construct
ARDRQQQPSPYYYHGMDV

SEQ ID NO: 84 - protein, 11 aa, synthetic construct
QASQDISNYLN

SEQ ID NO: 85 - protein, 8 aa, synthetic construct
QQYDNLPL

SEQ ID NO: 86 - protein, 125 aa, synthetic construct
EVQLVESGAEVKKPGSSVMVSCKASGGTFSRYAISWVRQAPGQGLEWMGGIIPVFGIVHYAQKFQGRVTITADKSTSTAYMELSSLRSEDTAAYYCARDRQQQPSPYYYHGMDVWGQGTTVTVSS

SEQ ID NO: 87 - protein, 106 aa, synthetic construct
DIRLTQSPSSLSASVGDRVTITCQASQDISNYLNWYQQKPGKAPKLLIYDASNLETGVPTRFSGSGSGTDFTFTISSLQPEDIATYYCQQYDNLPLFGGGTKVEIK

SEQ ID NO: 88 - protein, 9 aa, synthetic construct
LTVSSNYMS

SEQ ID NO: 89 - protein, 16 aa, synthetic construct
LIYSGGSTYYADSVKG

SEQ ID NO: 90 - protein, 13 aa, synthetic construct
ARGELGIPYGMDV

SEQ ID NO: 91 - protein, 13 aa, synthetic construct
SGGSSNVGDNFVS

SEQ ID NO: 92 - protein, 7 aa, synthetic construct
DNNRRPS

SEQ ID NO: 93 - protein, 11 aa, synthetic construct
GTWDSSLSAGV

SEQ ID NO: 94 - protein, 119 aa, synthetic construct
EVQLLESGGGLVQPGGSLRLSCAVSGLTVSSNYMSWVRQAPGKGLEWVSLIYSGGSTYYADSVKGRFTISRDNSKNTLYLQMNSLRAEDTAVYYCARGELGIPYGMDVWGQGTTVTVSS

SEQ ID NO: 95 - protein, 110 aa, synthetic construct
QSVLTQPPSVSAAPGQKVTISCSGGSSNVGDNFVSWYQQLPGTAPKLLIYDNNRRPSGIPDRFSGSKSGTSATLGITGLQTGDEADYYCGTWDSSLSAGVFGGGTKVTVL

SEQ ID NO: 96 - protein, 9 aa, synthetic construct
YTFTDYYMH

SEQ ID NO: 97 - protein, 17 aa, synthetic construct
WINPNSGGTNSAQKFQG

SEQ ID NO: 98 - protein, 15 aa, synthetic construct
ARDSHLAMVRGDFEY

SEQ ID NO: 99 - protein, 14 aa, synthetic construct
TGTSSDVESYNVVS

SEQ ID NO: 100 - protein, 7 aa, synthetic construct
EVSKRPS

SEQ ID NO: 101 - protein, 10 aa, synthetic construct
CSYAGNSTWV

SEQ ID NO: 102 - protein, 122 aa, synthetic construct
QVQLVQSGAEVKKPGASVKVSCKASGYTFTDYYMHWVRQAPGQGLEWMGWINPNSGGTNSAQKFQGRVTMTRDTSINTAYMELSRLRSDDTAVYFCARDSHLAMVRGDFEYWGQGTLVTVSS

SEQ ID NO: 103 - protein, 110 aa, synthetic construct
QSVLTQPASVSGSPGQSITISCTGTSSDVESYNVVSWYQQHPGKAPKLMIYEVSKRPSGVSHRFSGSKSGNTASLTISGLQAEDESDYYCCSYAGNSTWVFGGGTKLTVL

SEQ ID NO: 104 - protein, 9 aa, synthetic construct
FTFSTYGMH

SEQ ID NO: 105 - protein, 17 aa, synthetic construct
VIWYDGSNKYYADSVKG

SEQ ID NO: 106 - protein, 14 aa, synthetic construct
ARSVDGDYLEYFQH

SEQ ID NO: 107 - protein, 14 aa, synthetic construct
TGTSSDVGGYNYVS

SEQ ID NO: 108 - protein, 7 aa, synthetic construct
DVSKRPS

SEQ ID NO: 109 - protein, 11 aa, synthetic construct
CSYAGGYTNYV

SEQ ID NO: 110 - protein, 121 aa, synthetic construct
EVQLVESGGGVVQPGRSLRLSCAASGFTFSTYGMHWVRQAPGKGLEWVAVIWYDGSNKYYADSVKGRFTISRDNSKNTLYLQMNSLRAEDTAVYYCARSVDGDYLEYFQHWGQGTLVTVSS

SEQ ID NO: 111 - protein, 111 aa, synthetic construct
QPVLTQPRSVSGSPGQSVTISCTGTSSDVGGYNYVSWYQQHPGKAPKLMIYDVSKRPSGVPDRFSGSKSGNTASLTISGLQAEDEADYYCCSYAGGYTNYVFGTGTKLTVL

SEQ ID NO: 112 - protein, 9 aa, synthetic construct
YTSSSYYMH

SEQ ID NO: 113 - protein, 17 aa, synthetic construct
IINPGGTSTSYAQKFQG

SEQ ID NO: 114 - protein, 18 aa, synthetic construct
AIGYCSTTSCRWGDAFDI

SEQ ID NO: 115 - protein, 11 aa, synthetic construct
RASQSISSYLN

SEQ ID NO: 116 - protein, 9 aa, synthetic construct
QQSSTTPPT

SEQ ID NO: 117 - protein, 125 aa, synthetic construct
QVQLVQSGAEVKKPGASVKVSCKASGYTSSSYYMHWVRQAPGQGPEWMGIINPGGTSTSYAQKFQGRVTMTRDTSTSTVYMELSSLRSEDTAVYYCAIGYCSTTSCRWGDAFDIWGQGTMVTVSS

SEQ ID NO: 118 - protein, 107 aa, synthetic construct
DIQVTQSPSSLSASVGDRVTITCRASQSISSYLNWYQQKPEKAPNLLIYAASSLQSGVPSRFSGSGSGTDFTLTISSLQPEDFATYYCQQSSTTPPTFGQGTKVEIK

SEQ ID NO: 119 - protein, 9 aa, synthetic construct
FTFDDYAMH

SEQ ID NO: 120 - protein, 17 aa, synthetic construct
GISWNSGTIGYADSVKG

SEQ ID NO: 121 - protein, 16 aa, synthetic construct
GKDRKREDPSLGGMDV

SEQ ID NO: 122 - protein, 11 aa, synthetic construct
GGNNIGSKSVH

SEQ ID NO: 123 - protein, 7 aa, synthetic construct
DDTDRPS

SEQ ID NO: 124 - protein, 11 aa, synthetic construct
QVWDSSSDHYV

SEQ ID NO: 125 - protein, 123 aa, synthetic construct
QVQLVESGGGLVQPGRSLRLSCAASGFTFDDYAMHWVRQAPGKGLEWVSGISWNSGTIGYADSVKGRFIISRDNAKNSLYLQMNSLRGEDMALYYCGKDRKREDPSLGGMDVWGQGTTVTVSS

SEQ ID NO: 126 - protein, 108 aa, synthetic construct
SYELTQPPSVSVAPGQTARITCGGNNIGSKSVHWYQQKPGQAPVLVVYDDTDRPSGIPERFSGSNSGNTATLTISRVEAGDEADYYCQVWDSSSDHYVFGTGTKLTVL

SEQ ID NO: 127 - protein, 9 aa, synthetic construct
GSISSYYWS

SEQ ID NO: 128 - protein, 16 aa, synthetic construct
YIYYSGSTNYNPSLKS

SEQ ID NO: 129 - protein, 26 aa, synthetic construct
AKSASDEPPPSTAAAGSHYYYYGMDV

SEQ ID NO: 130 - protein, 9 aa, synthetic construct
MQALQTPNT

SEQ ID NO: 131 - protein, 132 aa, synthetic construct
EVQLQESGPGLVKPSETLSLTCTVSGGSISSYYWSWIRQPPGKGLEWIGYIYYSGSTNYNPSLKSRVTISVGTSKNQFSLKLSSVTAADTAVYYCAKSASDEPPPSTAAAGSHYYYYGMDVWGQGTTVTVSS

SEQ ID NO: 132 - protein, 112 aa, synthetic construct
EIVMTQSPLSLPVTPGEPASISCRSSQSLLHSNGYNYLDWYLQKPGQSPHVLIYLGSNRASGVPDRFSGSGSGTDFTLKISRVEAEDVGVYYCMQALQTPNTFGQGTKLEIK

SEQ ID NO: 133 - protein, 9 aa, synthetic construct
FTFSRYWMH

SEQ ID NO: 134 - protein, 17 aa, synthetic construct
RINSDGSSTTYADSVKG

SEQ ID NO: 135 - protein, 21 aa, synthetic construct
ARELYYYDSRGAEEGEGWFDP

SEQ ID NO: 136 - protein, 14 aa, synthetic construct
TGISSNIGAGYDVH

SEQ ID NO: 137 - protein, 7 aa, synthetic construct
GNSNRPS

SEQ ID NO: 138 - protein, 11 aa, synthetic construct
QSSDRSLSGSV

SEQ ID NO: 139 - protein, 128 aa, synthetic construct
EVQLLESGGGLVQPGGSLRLSCAASGFTFSRYWMHWVRQAPGKGLVWVSRINSDGSSTTYADSVKGRFTISRDNAKNTLYLQMNSLRAEDTAVYYCARELYYYDSRGAEEGEGWFDPWGQGTLVTVSS

SEQ ID NO: 140 - protein, 111 aa, synthetic construct
QSVLTQPPSVSGAPGQRVTISCTGISSNIGAGYDVHWYQQLPGTAPKLLIYGNSNRPSGVPDRFSASKSGTSAPLAITGLQAEDEADYYCQSSDRSLSGSVFGTGTKLTVL

SEQ ID NO: 141 - protein, 9 aa, synthetic construct
YSFTSYWIA

SEQ ID NO: 142 - protein, 17 aa, synthetic construct
IIYPGDSDTSYSPSFQG

SEQ ID NO: 143 - protein, 12 aa, synthetic construct
ARGPNKYNWFDT

SEQ ID NO: 144 - protein, 13 aa, synthetic construct
SGSSSNIGSNTVN

SEQ ID NO: 145 - protein, 7 aa, synthetic construct
SNNQRPS

SEQ ID NO: 146 - protein, 11 aa, synthetic construct
AAWDDSLNGVV

SEQ ID NO: 147 - protein, 119 aa, synthetic construct
QVQLVQSGAEVKKPGESLKISCKGSGYSFTSYWIAWVRQMPGKGLEWMGIIYPGDSDTSYSPSFQGQVTISADKSISTAYLQWSSLKASDTAMYYCARGPNKYNWFDTWGQGTLVTVSS

SEQ ID NO: 148 - protein, 110 aa, synthetic construct
SYVLTQPPSASGTPGQRVTISCSGSSSNIGSNTVNWYQQLPGTAPKLLIYSNNQRPSGVPDRFSGSKSGTSASLAISGLQSEDETDYYCAAWDDSLNGVVFGGGTKLTVL

SEQ ID NO: 149 - protein, 127 aa, synthetic construct
QVQLVESGGGVVQPGRSLRLSCAASGFTFSNFAMHWVRQAPGKGLEWVAVILYDGINKYYADSVKGRFTISRGNSKNTLSLQMNSLRAEDTAVYYCARAQNYYDRSGTLQLDAFDIWGQGTLVTVSS

SEQ ID NO: 150 - protein, 107 aa, synthetic construct
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIK

SEQ ID NO: 151 - protein, 11 aa, synthetic construct
VASQGIRNDLG

SEQ ID NO: 152 - protein, 7 aa, synthetic construct
EASSLES

SEQ ID NO: 153 - protein, 107 aa, synthetic construct
AIQMTQSPSSLSASVGDRVTITCVASQGIRNDLGWYQQKPGKAPKLLIYEASSLESGVPSRFSGSGSGTDFTLTISSLQPEDFATYYCLQDYNYPLTFGGGTKVEIK

SEQ ID NO: 154 - protein, 9 aa, synthetic construct
FTFDNFAMH

SEQ ID NO: 155 - protein, 17 aa, synthetic construct
VIEYDGINKYYADSVKG

SEQ ID NO: 156 - protein, 20 aa, synthetic construct
ARAQNYYDRYGTLQLDAFDI

SEQ ID NO: 157 - protein, 7 aa, synthetic construct
DASSLQS

SEQ ID NO: 158 - protein, 127 aa, synthetic construct
QVQLVQSGGGVVQPGRSLRLSCAASGFTFDNFAMHWVRQAPGKGLEWVAVIEYDGINKYYADSVKGRFTISRGNSKNTLSLQMNSLRAEDTAVYYCARAQNYYDRYGTLQLDAFDIWGQGTLVTVSS

SEQ ID NO: 159 - protein, 107 aa, synthetic construct
AIQMTQSPSSLSASVGDRVTITCRASQGIRNDLGWYQQKPGKAPKLLIYDASSLQSGVPSRFSGSGSGTDFTLTISSLQPEDFATYYCLQDYNYPLTFGGGTKVEIK

SEQ ID NO: 160 - protein, 17 aa, synthetic construct
VILYDGINDYYADSVKG

SEQ ID NO: 161 - protein, 20 aa, synthetic construct
ARAQNYYDRHGTLQLDAFDI

SEQ ID NO: 162 - protein, 11 aa, synthetic construct
RASDGIRNDLG

SEQ ID NO: 163 - protein, 7 aa, synthetic construct
EASSLQS

SEQ ID NO: 164 - protein, 9 aa, synthetic construct
LQNYNYPLT

SEQ ID NO: 165 - protein, 127 aa, synthetic construct
QVQLVQSGGGVVQPGRSLRLSCAASGFTFDNFAMHWVRQAPGKGLEWVAVILYDGINDYYADSVKGRFTISRGNSKNTLSLQMNSLRAEDTAVYYCARAQNYYDRHGTLQLDAFDIWGQGTLVTVSS

SEQ ID NO: 166 - protein, 107 aa, synthetic construct
AIQMTQSPSSLSASVGDRVTITCRASDGIRNDLGWYQQKPGKAPKLLIYEASSLQSGVPSRFSGSGSGTDFTLTISSLQPEDFATYYCLQNYNYPLTFGGGTKVEIK

SEQ ID NO: 167 - protein, 128 aa, synthetic construct
EVQLVESGGGLVQPGGSLRLSCAASGFTFSRYWMHWVRQAPGKGLVWVSRINSDGSSTTYADSVKGRFTISRDNAKNTLYLQMNSLRAEDTAVYYCARELYYYDSRGAEEGEGWFDPWGQGTLVTVSS

SEQ ID NO: 168 - protein, 111 aa, synthetic construct
QSVLTQPPSVSGAPGQRVTISCTGISSNIGAGYDVHWYQQLPGTAPKLLIYGNSNRPSGVPDRFSASKSGTSAPLAITGLQAEDEADYYCQSSDRSLSGSVFGTGTKVTVL

SEQ ID NO: 169 - protein, 9 aa, synthetic construct
FTFRRYWMH

SEQ ID NO: 170 - protein, 21 aa, synthetic construct
ARELYYYDSRGFEEGEGWFDP

SEQ ID NO: 171 - protein, 128 aa, synthetic construct
EVQLVESGGGLVQPGGSLRLSCAASGFTFRRYWMHWVRQAPGKGLVWVSRINSDGSSTTYADSVKGRFTISRDNAKNTLYLQMNSLRAEDTAVYYCARELYYYDSRGFEEGEGWFDPWGQGTLVTVSS

SEQ ID NO: 172 - protein, 14 aa, synthetic construct
TGISSNIGAQYDVH

SEQ ID NO: 173 - protein, 7 aa, synthetic construct
GNSNRPR

SEQ ID NO: 174 - protein, 11 aa, synthetic construct
QSSDRSVSGSV

SEQ ID NO: 175 - protein, 111 aa, synthetic construct
QSVLTQPPSVSGAPGQRVTISCTGISSNIGAQYDVHWYQQLPGTAPKLLIYGNSNRPRGVPDRFSASKSGTSAPLAITGLQAEDEADYYCQSSDRSVSGSVFGTGTKVTVL

SEQ ID NO: 176 - protein, 17 aa, synthetic construct
RINSDGSSTTYADSVEG

SEQ ID NO: 177 - protein, 21 aa, synthetic construct
ARELYYYDRRGAEEGEGWFDP

SEQ ID NO: 178 - protein, 14 aa, synthetic construct
TGISSNIGAEYDVH

SEQ ID NO: 179 - protein, 11 aa, synthetic construct
QVSDRYLSGSV

SEQ ID NO: 180 - protein, 128 aa, synthetic construct
EVQLVESGGGLVQPGGSLRLSCAASGFTFRRYWMHWVRQAPGKGLEWVSRINSDGSSTTYADSVEGRFTISRDNAKNTLYLQMNSLRAEDTAVYYCARELYYYDRRGAEEGEGWFDPWGQGTLVTVSS

SEQ ID NO: 181 - protein, 111 aa, synthetic construct
QSVLTQPPSVSGAPGQRVTISCTGISSNIGAEYDVHWYQQLPGTAPKLLIYGNSNRPRGVPDRFSASKSGTSAPLAITGLQAEDEADYYCQVSDRYLSGSVFGTGTKVTVL

SEQ ID NO: 182 - protein, 9 aa, synthetic construct
FTFSSYDMS

SEQ ID NO: 183 - protein, 17 aa, synthetic construct
GISGSGAITYYTDSVKG

SEQ ID NO: 184 - protein, 12 aa, synthetic construct
TKENNRNTFFDY

SEQ ID NO: 185 - protein, 11 aa, synthetic construct
RASQTVRSDLA

SEQ ID NO: 186 - protein, 12 aa, synthetic construct
QQYYEWPPHSDS

SEQ ID NO: 187 - protein, 119 aa, synthetic construct
EVQLVESGGGVVRPGESLRLSCAASGFTFSSYDMSWVRQAPGEGLEWVSGISGSGAITYYTDSVKGRFTISRDNSKNTLYLQMNSLRAEDTAVYYCTKENNRNTFFDYWGQGTLVTVSS

SEQ ID NO: 188 - protein, 110 aa, synthetic construct
EIVLTQSPGTLSLSPGERATLSCRASQTVRSDLAWYQQKPGQAPRLLIYGASTRATDISDRFSGSGSGTEFTLTISSLQSEDFAVYYCQQYYEWPPHSDSFGGGTKVEIK

SEQ ID NO: 189 - protein, 9 aa, synthetic construct
FTFSSTDMS

SEQ ID NO: 190 - protein, 12 aa, synthetic construct
TKENNRATFFDY

SEQ ID NO: 191 - protein, 119 aa, synthetic construct
EVQLVESGGGVVRPGESLRLSCAASGFTFSSTDMSWVRQAPGEGLEWVSGISGSGAITYYTDSVKGRFTISRDNSKNTLYLQMNSLRAEDTAVYYCTKENNRATFFDYWGQGTLVTVSS

SEQ ID NO: 192 - protein, 9 aa, synthetic construct
PTFSSYDMS

SEQ ID NO: 193 - protein, 12 aa, synthetic construct
TKENNRGTFFDY

SEQ ID NO: 194 - protein, 11 aa, synthetic construct
SASQTVRSDLA

SEQ ID NO: 195 - protein, 7 aa, synthetic construct
GGSTRAT

SEQ ID NO: 196 - protein, 119 aa, synthetic construct
EVQLVESGGGVVRPGESLRLSCAASGPTFSSYDMSWVRQAPGEGLEWVSGISGSGAITYYTDSVKGRFTISRDNSKNTLYLQMNSLRAEDTAVYYCTKENNRGTFFDYWGQGTLVTVSS

SEQ ID NO: 197 - protein, 110 aa, synthetic construct
EIVLTQSPGTLSLSPGERATLSCSASQTVRSDLAWYQQKPGQAPRLLIYGGSTRATDISDRFSGSGSGTEFTLTISSLQSEDFAVYYCQQYYEWPPHSDSFGGGTKVEIK

SEQ ID NO: 198 - protein, 17 aa, synthetic construct
GISGEGAITYYTDSVKG

SEQ ID NO: 199 - protein, 119 aa, synthetic construct
EVQLVESGGGVVRPGESLRLSCAASGFTFSSYDMSWVRQAPGEGLEWVSGISGEGAITYYTDSVKGRFTISRDNSKNTLYLQMNSLRAEDTAVYYCTKENNRATFFDYWGQGTLVTVSS

SEQ ID NO: 200 - protein, 110 aa, synthetic construct
EIVLTQSPGTLSLSPGERATLSCRASQTVRSDLAWYQQKPGQAPRLLIYGGSTRATDISDRFSGSGSGTEFTLTISSLQSEDFAVYYCQQYYEWPPHSDSFGGGTKVEIK

SEQ ID NO: 201 - protein, 123 aa, synthetic construct
EVQLVESGGGLVQPGRSLRLSCAASGFTFDDYAMHWVRQAPGKGLEWVSGISWNSGTIGYADSVKGRFIISRDNAKNSLYLQMNSLRGEDMALYYCGKDRKREDPSLGGMDVWGQGTTVTVSS

SEQ ID NO: 202 - protein, 108 aa, synthetic construct
SYVLTQPPSVSVAPGQTARITCGGNNIGSKSVHWYQQKPGQAPVLVVYDDTDRPSGIPERFSGSNSGNTATLTISRVEAGDEADYYCQVWDSSSDHYVFGTGTKVTVL

SEQ ID NO: 203 - protein, 9 aa, synthetic construct
FTIDDYAMH

SEQ ID NO: 204 - protein, 17 aa, synthetic construct
GISWNGGTIGYADSVKG

SEQ ID NO: 205 - protein, 11 aa, synthetic construct
LGNNIGSKSVH

SEQ ID NO: 206 - protein, 11 aa, synthetic construct
QVWDSESDHYV

SEQ ID NO: 207 - protein, 123 aa, synthetic construct
EVQLVESGGGLVQPGRSLRLSCAASGFTIDDYAMHWVRQAPGKGLEWVSGISWNGGTIGYADSVKGRFIISRDNAKNSLYLQMNSLRGEDMALYYCGKDRKREDPSLGGMDVWGQGTTVTVSS

SEQ ID NO: 208 - protein, 108 aa, synthetic construct
SYVLTQPPSVSVAPGQTARITCLGNNIGSKSVHWYQQKPGQAPVLVVYDDTDRPSGIPERFSGSNSGNTATLTISRVEAGDEADYYCQVWDSESDHYVFGTGTKVTVL

SEQ ID NO: 209 - protein, 17 aa, synthetic construct
GTSWNSGTIGYADSVKG

SEQ ID NO: 210 - protein, 16 aa, synthetic construct
VKDRKREDPSLGGMDV

SEQ ID NO: 211 - protein, 11 aa, synthetic construct
QVWDVSSDSYV

SEQ ID NO: 212 - protein, 123 aa, synthetic construct
EVQLVESGGGLVQPGRSLRLSCAASGFTFDDYAMHWVRQAPGKGLEWVSGTSWNSGTIGYADSVKGRFIISRDNAKNSLYLQMNSLRGEDMALYYCVKDRKREDPSLGGMDVWGQGTTVTVSS

SEQ ID NO: 213 - protein, 108 aa, synthetic construct
SYVLTQPPSVSVAPGQTARITCGGNNIGSKSVHWYQQKPGQAPVLVVYDDTDRPSGIPERFSGSNSGNTATLTISRVEAGDEADYYCQVWDVSSDSYVFGTGTKVTVL

SEQ ID NO: 214 - protein, 11 aa, synthetic construct
QVWDSSSDDYV

SEQ ID NO: 215 - protein, 108 aa, synthetic construct
SYVLTQPPSVSVAPGQTARITCGGNNIGSKSVHWYQQKPGQAPVLVVYDDTDRPSGIPERFSGSNSGNTATLTISRVEAGDEADYYCQVWDSSSDDYVFGTGTKVTVL

SEQ ID NO: 216 - protein, 107 aa, synthetic construct
DIQMTQSPSSLSASVGDRVTITCQASQDIKKYLNWYQQKPGKAPKLLIYDVSNLETGVPSRFSGSGSGTDFTFTISSLQPEDVAIYYCQQYDNLPQTFGQGTKVEIK

SEQ ID NO: 217 - protein, 9 aa, synthetic construct
FTFADYTMS

SEQ ID NO: 218 - protein, 19 aa, synthetic construct
FIRSEPYGGTTEYAASVKG

SEQ ID NO: 219 - protein, 131 aa, synthetic construct
EVQLVESGGGLVKPGRSLRLSCAASGFTFADYTMSWFRQAPGKGLEWVGFIRSEPYGGTTEYAASVKGRFTISRDDSKSIAYLQMNSLKTEDTAVYYCSRDPWYCSGGDCYAVTGSWFDPWGQGTLVTVSS

SEQ ID NO: 220 - protein, 11 aa, synthetic construct
QASQDIKQYLN

SEQ ID NO: 221 - protein, 7 aa, synthetic construct
DQSNLET

SEQ ID NO: 222 - protein, 9 aa, synthetic construct
QQYWNLPQT

SEQ ID NO: 223 - protein, 107 aa, synthetic construct
DIQMTQSPSSLSASVGDRVTITCQASQDIKQYLNWYQQKPGKAPKLLIYDQSNLETGVPSRFSGSGSGTDFTFTISSLQPEDVAIYYCQQYWNLPQTFGQGTKVEIK

SEQ ID NO: 224 - protein, 11 aa, synthetic construct
QASRDIKKYLN

SEQ ID NO: 225 - protein, 9 aa, synthetic construct
QQYDQLPQT

SEQ ID NO: 226 - protein, 107 aa, synthetic construct
DIQMTQSPSSLSASVGDRVTITCQASRDIKKYLNWYQQKPGKAPKLLIYDVSNLETGVPSRFSGSGSGTDFTFTISSLQPEDVAIYYCQQYDQLPQTFGQGTKVEIE

SEQ ID NO: 227 - protein, 1273 aa, unidentified
  source 1..1273: note = SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 228 - protein, 330 aa, Homo sapiens [sequence omitted]

SEQ ID NO: 229 - protein, 107 aa, synthetic construct [sequence omitted]

SEQ ID NO: 230 - protein, 9 aa, synthetic construct
  VARIANT 4: note = any one of S, T, D or E
FTFXNFAMH

SEQ ID NO: 231 - protein, 17 aa, synthetic construct
  VARIANT 3: note = any one of A, L, I, V, E, or D,
  VARIANT 9: note = any one of K, R, D or E
VIXYDGINXYYADSVKG

SEQ ID NO: 232 - protein, 20 aa, synthetic construct
  VARIANT 10: note = any one of S, T, Y, H
ARAQNYYDRXGTLQLDAFDI

SEQ ID NO: 233 - protein, 11 aa, synthetic construct
  VARIANT 1: note = any one of R, K, V, L, A, or I
  VARIANT 4: note = any one of Q, N, E or D
XASXGIRNDLG

SEQ ID NO: 234 - protein, 7 aa, synthetic construct
  VARIANT 1: note = any one of A, V, L, I, E or D
  VARIANT 6: note = any one of Q, N, D or E
XASSLXS

SEQ ID NO: 235 - protein, 9 aa, synthetic construct
  VARIANT 3: note = any one of N, Q, E or D
LQXYNYPLT

SEQ ID NO: 236 - protein, 9 aa, synthetic construct
  VARIANT 4: note = any one of S or D
FTFXNFAMH

SEQ ID NO: 237 - protein, 17 aa, synthetic construct
  VARIANT 3: note = any one of L or E
  VARIANT 9: note = any one of K or D
VIXYDGINXYYADSVKG

SEQ ID NO: 238 - protein, 20 aa, synthetic construct
  VARIANT 10: note = any one of S, Y, or H
ARAQNYYDRXGTLQLDAFDI

SEQ ID NO: 239 - protein, 11 aa, synthetic construct
  VARIANT 1: note = any one of R or V
  VARIANT 4: note = any one of Q or D
XASXGIRNDLG

SEQ ID NO: 240 - protein, 7 aa, synthetic construct
  VARIANT 1: note = any one of A, E or D
  VARIANT 6: note = any one of Q or E
XASSLXS

SEQ ID NO: 241 - protein, 9 aa, synthetic construct
  VARIANT 3: note = any one of N or D
LQXYNYPLT

SEQ ID NO: 242 - protein, 9 aa, synthetic construct
  VARIANT 4: note = any one of S, T, K or R
FTFXRYWMH

SEQ ID NO: 243 - protein, 17 aa, synthetic construct
  VARIANT 16: note = any one of K, R, D or E
RINSDGSSTTYADSVXG

SEQ ID NO: 244 - protein, 21 aa, synthetic construct
  VARIANT 9: note = any one of S, T, K or R
  VARIANT 12: note = any one of A, V, I, L, W or F
ARELYYYDXRGXEEGEGWFDP

SEQ ID NO: 245 - protein, 14 aa, synthetic construct
  VARIANT 10: note = any one of G, N, Q, D, or E
TGISSNIGAXYDVH

SEQ ID NO: 246 - protein, 7 aa, synthetic construct
  VARIANT 7: note = any one of S, T, K or R
GNSNRPX

SEQ ID NO: 247 - protein, 11 aa, synthetic construct
  VARIANT 2: note = any one of S, T, A, I, L or V,
  VARIANT 6: note = any one of S, T, or Y
  VARIANT 7: note = any one of S, T, or Y
QXSDRXXSGSV

SEQ ID NO: 248 - protein, 9 aa, synthetic construct
  VARIANT 4: note = any one of S or R
FTFXRYWMH

SEQ ID NO: 249 - protein, 17 aa, synthetic construct
  VARIANT 16: note = any one of K or E
RINSDGSSTTYADSVXG

SEQ ID NO: 250 - protein, 21 aa, synthetic construct
  VARIANT 9: note = any one of S or R
  VARIANT 12: note = any one of A or F
ARELYYYDXRGXEEGEGWFDP

SEQ ID NO: 251 - protein, 14 aa, synthetic construct
  VARIANT 10: note = any one of G, Q, or E
TGISSNIGAXYDVH

SEQ ID NO: 252 - protein, 7 aa, synthetic construct
  VARIANT 7: note = any one of S or R
GNSNRPX

SEQ ID NO: 253 - protein, 11 aa, synthetic construct
  VARIANT 2: note = any one of S or V
  VARIANT 6: note = any one of S or Y
  VARIANT 7: note = any one of L or V
QXSDRXXSGSV

SEQ ID NO: 254 - protein, 9 aa, synthetic construct
  VARIANT 1: note = any one of F, W or P
  VARIANT 6: note = any one of Y, T or S
XTFSSXDMS

SEQ ID NO: 255 - protein, 17 aa, synthetic construct
  VARIANT 5: note = any one of S, T, D or E
GISGXGAITYYTDSVKG

SEQ ID NO: 256 - protein, 12 aa, synthetic construct
  VARIANT 7: note = any one of N, Q, A, L, I, V, or G
TKENNRXTFFDY

SEQ ID NO: 257 - protein, 11 aa, synthetic construct
  VARIANT 1: note = any one of R, K, S or T
XASQTVRSDLA

SEQ ID NO: 258 - protein, 7 aa, synthetic construct
  VARIANT 2: note = any one of A, V, L, I or G
GXSTRAT

SEQ ID NO: 259 - protein, 9 aa, synthetic construct
  VARIANT 1: note = any one of F or P
  VARIANT 6: note = any one of Y or T
XTFSSXDMS

SEQ ID NO: 260 - protein, 17 aa, synthetic construct
  VARIANT 5: note = any one of S or E
GISGXGAITYYTDSVKG

SEQ ID NO: 261 - protein, 12 aa, synthetic construct
  VARIANT 7: note = any one of N, A, or G
TKENNRXTFFDY

SEQ ID NO: 262 - protein, 11 aa, synthetic construct
  VARIANT 1: note = any one of R or S
XASQTVRSDLA

SEQ ID NO: 263 - protein, 7 aa, synthetic construct
  VARIANT 2: note = any one of A or G
GXSTRAT

SEQ ID NO: 264 - protein, 9 aa, synthetic construct
  VARIANT 3: note = any one of F, W, I, or L
FTXDDYAMH

SEQ ID NO: 265 - protein, 17 aa, synthetic construct
  VARIANT 2: note = any one of I, L, V, A, S or T
  VARIANT 6: note = any one of S, T or G
GXSWNXGTIGYADSVKG

SEQ ID NO: 266 - protein, 16 aa, synthetic construct
  VARIANT 1: note = any one of G I, L, A or V
XKDRKREDPSLGGMDV

SEQ ID NO: 267 - protein, 11 aa, synthetic construct
  VARIANT 1: note = any one of G, L, I, A or V
XGNNIGSKSVH

SEQ ID NO: 268 - protein, 11 aa, synthetic construct
  VARIANT 5: note = any one of S, T, I, L, A or V
  VARIANT 6: note = any one of S, T, D or E
  VARIANT 9: note = any one of H, S, T, E or D
QVWDXXSDXYV

SEQ ID NO: 269 - protein, 9 aa, synthetic construct
  VARIANT 3: note = any one of F or I
FTXDDYAMH

SEQ ID NO: 270 - protein, 17 aa, synthetic construct
  VARIANT 2: note = any one of I or T
  VARIANT 6: note = any one of S or G
GXSWNXGTIGYADSVKG

SEQ ID NO: 271 - protein, 16 aa, synthetic construct
  VARIANT 1: note = any one of G or V
XKDRKREDPSLGGMDV

SEQ ID NO: 272 - protein, 11 aa, synthetic construct
  VARIANT 1: note = any one of G or L
XGNNIGSKSVH

SEQ ID NO: 273 - protein, 11 aa, synthetic construct
  VARIANT 5: note = any one of S or V
  VARIANT 6: note = any one of S or E
  VARIANT 9: note = any one of H, S, or D
QVWDXXSDXYV

SEQ ID NO: 274 - protein, 9 aa, synthetic construct
  VARIANT 7: note = any one of A, V, S or T
FTFADYXMS

SEQ ID NO: 275 - protein, 19 aa, synthetic construct
  VARIANT 5: note = any one of K, R, E, or D
FIRSXPYGGTTEYAASVKG

SEQ ID NO: 276 - protein, 11 aa, synthetic construct
  VARIANT 4: note = any one of Q, N, K or R
  VARIANT 8: note = any one of K, R, N, or Q
QASXDIKXYLN

SEQ ID NO: 277 - protein, 7 aa, synthetic construct
  VARIANT 2: note = any one of V, I, L, A, N or Q
DXSNLET

SEQ ID NO: 278 - protein, 9 aa, synthetic construct
  VARIANT 4: note = any one of D, E, F or W
  VARIANT 5: note = any one of N or Q
QQYXXLPQT

SEQ ID NO: 279 - protein, 9 aa, synthetic construct
  VARIANT 7: note = any one of A or T
FTFADYXMS

SEQ ID NO: 280 - protein, 19 aa, synthetic construct
  VARIANT 5: note = any one of K or E
FIRSXPYGGTTEYAASVKG

SEQ ID NO: 281 - protein, 11 aa, synthetic construct
  VARIANT 4: note = any one of Q or R
  VARIANT 8: note = any one of K or Q
QASXDIKXYLN

SEQ ID NO: 282 - protein, 7 aa, synthetic construct
  VARIANT 2: note = any one of V or Q
DXSNLET

SEQ ID NO: 283 - protein, 9 aa, synthetic construct
  VARIANT 4: note = any one of D or W
  VARIANT 5: note = any one of N or Q
QQYXXLPQT

(19) United States
(12) Patent Application Publication
(10) Pub. No.: US 2026/0217797 A1
(43) Pub. Date: Jul. 30, 2026

(54) CORONAVIRUS ANTIBODIES AND THERAPEUTIC USES THEREOF

(71) Applicant: Takeda Pharmaceutical Company Limited, Osaka (JP)

(72) Inventors: Jerry Thomas, San Diego, CA (US); Weihong Yu, San Diego, CA (US); Dianna Lundberg, Brentwood, NH (US); David Bouck, Arlington, MA (US); Stefan Koester, Herne (DE); Kurt Y. Eng, Northborough, MA (US); James Geoghegan, Lebanon, NH (US); C. Garrett Rappazzo, Lebanon, NH (US); Caitlin Stein, Lebanon, NH (US); Rebekah Costello, Lebanon, NH (US); Shelley Izquierdo, Berkley, CA (US); William D. Harriman, Alameda, CA (US)

(73) Assignee: Takeda Pharmaceutical Company Limited, Osaka (JP)

(21) Appl. No.: 19/147,605

(22) PCT Filed: Jan. 12, 2024
(86) PCT No.: PCT/IB2024/000062

§ 371 Date: Jul. 11, 2025

Related U.S. Application Data
(60) Provisional application No. 63/439,034, filed on Jan. 13, 2023.

(51) Int. Cl. C07K 16/104; A61K 31/403; A61K 31/427; A61K 31/7068; A61K 39/00; A61P 31/14

(52) U.S. Cl. CPC C07K 16/104; A61K 31/403; A61K 31/427; A61K 31/7068; A61P 31/14; A61K 2039/505; C07K 2317/24; C07K 2317/52; C07K 2317/55; C07K 2317/565; C07K 2317/76; C07K 2317/92

(57) ABSTRACT

Disclosed herein are antibodies that bind to coronavirus spike protein (e.g., SARS-CoV-2 spike protein), and methods of use thereof (e.g., treatment of subjects infected with SARSCoV-2).

### CROSS-REFERENCE TO RELATED

This application claims the benefit under 35 U.S.C. § 119(e) of U.S. provisional application No. 63/439,034, filed Jan. 13, 2023, the entire contents of which are incorporated herein by reference.

### REFERENCE TO AN ELECTRONIC SEQUENCE LISTING

The contents of the electronic sequence listing (T083370007WO00-SEQ-ROS.xml; Size: 285,663 bytes; and Date of Creation: Jan. 11, 2024) is herein incorporated by reference in its entirety.

### BACKGROUND

Since the COVID-19 (Coronavirus Disease 2019) outbreak, caused by the severe acute respiratory syndrome coronavirus 2 (SARS-CoV-2) virus, more than 535 million cases of COVID-19 have been confirmed, resulting in over 6.3 million deaths. Most currently available SARS-CoV-2 antibodies are ineffective against known and emerging SARS-CoV-2 variants with spike protein mutations.

### SUMMARY

In some aspects, the present disclosure provides antibodies that bind to coronavirus spike protein (e.g., SARS-CoV-2 spike protein), and methods of use thereof (e.g., treatment of SARS-CoV-2 infection). In some embodiments, the antibodies described herein bind to and/or neutralize various SARS-CoV-2 strains as well as related coronavirus family members, including: SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, SARS-CoV-2 Omicron, SARS-CoV-1, and WIV.

In some aspects, this disclosure provides an antibody that binds to a severe acute respiratory syndrome coronavirus 2 (SARS-CoV-2) spike protein, wherein the antibody comprises: a heavy chain variable domain (VH) comprising a heavy chain complementary determining region 1 (CDR-H1) comprising the amino acid sequence of FTFX₁NFAMH (SEQ ID NO: 230), wherein X₁ is S, T, D or E; a heavy chain complementary determining region 2 (CDR-H2) comprising the amino acid sequence of VIX₂YDGINX₃YYADSVKG (SEQ ID NO: 231), wherein X₂ is A, L, I, V, E, or D, and X₃ is K, R, D or E; a heavy chain complementary determining region 3 (CDR-H3) comprising the amino acid sequence of ARAQNYYDRX₄GTLQLDAFDI (SEQ ID NO: 232), wherein X₄ is S. T, Y, H; and/or a light chain variable domain (VL) comprising a light chain complementary determining region 1 (CDR-L1) comprising the amino acid sequence of X₅ASX₆GIRNDLG (SEQ ID NO: 233), wherein X₅ is R, K, V, L, A, or I and X₆ is Q, N, E or D; a light chain complementary determining region 1 (CDR-L2) comprising the amino acid sequence of X₇ASSLX₈S (SEQ ID NO: 234), wherein X₇ is A, V, L, I, E or D, and X₈ is Q, N, D or E; and a light chain complementary determining region 1 (CDR-L3) comprising the amino acid sequence of LQX₉YNYPLT (SEQ ID NO: 235), wherein X₉ is N, Q. E or D.

In some embodiments, the CDR-H1 comprises the amino acid sequence of FTFX₁NFAMH (SEQ ID NO: 236), wherein X₁ is S or D; the CDR-H2 comprises the amino acid sequence of VIX₂YDGINX₃YYADSVKG (SEQ ID NO: 237), wherein X₂ is L or E, and X₃ is K or D; the CDR-H3 comprises the amino acid sequence of ARAQNYYDRX₄GTLQLDAFDI (SEQ ID NO: 238), wherein X₄ is S. Y, or H; and/or the CDR-L1 comprises the amino acid sequence of X₅ASX₆GIRNDLG (SEQ ID NO: 239), wherein X₅ is R or V and X₆ is Q or D; the CDR-L2 comprises the amino acid sequence of X₇ASSLX₈S (SEQ ID NO: 240), wherein X₇ is A, E or D, and X₈ is Q or E; and the CDR-L3 comprises the amino acid sequence of LQX₉YNYPLT (SEQ ID NO: 241), wherein X₉ is N or D.

In some embodiments, the CDR-H1 comprises the amino acid sequence of SEQ ID NO: 41, the CDR-H2 comprises the amino acid sequence of SEQ ID NO: 42, the CDR-H3 comprises the amino acid sequence of SEQ ID NO: 43; and/or the CDR-L1 comprises the amino acid sequence of SEQ ID NO: 44, the CDR-L2 comprises the amino acid sequence of SEQ ID NO: 45, and the CDR-L3 comprises the amino acid sequence of SEQ ID NO: 46.

In some embodiments, the VH comprises the amino acid sequence of SEQ ID NO: 149, and the VL comprises the amino acid sequence of SEQ ID NO: 150.

In some embodiments, the VH comprises the amino acid sequence of SEQ ID NO: 47, and the VL comprises the amino acid sequence of SEQ ID NO: 48.

In some embodiments, the CDR-H1 comprises the amino acid sequence of SEQ ID NO: 41, the CDR-H2 comprises the amino acid sequence of SEQ ID NO: 42, the CDR-H3 comprises the amino acid sequence of SEQ ID NO: 43; and/or the CDR-L1 comprises the amino acid sequence of SEQ ID NO: 151, the CDR-L2 comprises the amino acid sequence of SEQ ID NO: 152, and the CDR-L3 comprises the amino acid sequence of SEQ ID NO: 46.

In some embodiments, the VH comprises the amino acid sequence of SEQ ID NO: 149, and the VL comprises the amino acid sequence of SEQ ID NO: 153.

In some embodiments, the CDR-H1 comprises the amino acid sequence of SEQ ID NO: 154, the CDR-H2 comprises the amino acid sequence of SEQ ID NO: 155, the CDR-H3 comprises the amino acid sequence of SEQ ID NO: 156; and/or the CDR-L1 comprises the amino acid sequence of SEQ ID NO: 44, the CDR-L2 comprises the amino acid sequence of SEQ ID NO: 157, and the CDR-L3 comprises the amino acid sequence of SEQ ID NO: 46.

In some embodiments, the VH comprises the amino acid sequence of SEQ ID NO: 158, and the VL comprises the amino acid sequence of SEQ ID NO: 159.

In some embodiments, the CDR-H1 comprises the amino acid sequence of SEQ ID NO: 154, the CDR-H2 comprises the amino acid sequence of SEQ ID NO: 160, the CDR-H3 comprises the amino acid sequence of SEQ ID NO: 161; and/or the CDR-L1 comprises the amino acid sequence of SEQ ID NO: 162, the CDR-L2 comprises the amino acid sequence of SEQ ID NO: 163, and the CDR-L3 comprises the amino acid sequence of SEQ ID NO: 164.

In some embodiments, the VH comprises the amino acid sequence of SEQ ID NO: 165, and the VL comprises the amino acid sequence of SEQ ID NO: 166.

In some embodiments, the antibody comprises: a VH comprising a CDR-H1 comprising the amino acid sequence of FTFX₁RYWMH (SEQ ID NO: 242), wherein X₁ is S, T, K or R; a CDR-H2 comprising the amino acid sequence of RINSDGSSTTYADSVX₂G (SEQ ID NO: 243), wherein X₂ is K, R, D or E; a CDR-H3 comprising the amino acid sequence of ARELYYYDX₃RGX₄EEGEGWFDP (SEQ ID NO: 244), wherein X₃ is S, T, K and R, and X₄ is A, V, I, L, W or F; and/or a VL comprising a CDR-L1 comprising the amino acid sequence of TGISSNIGAX₅YDVH (SEQ ID NO: 245), wherein X₅ is G, N, Q, D, or E; a CDR-L2 comprising the amino acid sequence of GNSNRPX₆ (SEQ ID NO: 246), wherein X₆ is S, T, K or R; and a CDR-L3 comprising the amino acid sequence of QX₇SDRX₈X₉SGSV (SEQ ID NO: 247), wherein X₇ is S, T, A, I, L or V, and X₈ is S, T, or Y, and X₉ is L, I, or V.

In some embodiments, the CDR-H1 comprises the amino acid sequence of FTFX₁RYWMH (SEQ ID NO: 248), wherein X₁ is S or R; the CDR-H2 comprises the amino acid sequence of RINSDGSSTTYADSVX₂G (SEQ ID NO: 249), wherein X₂ is K or E; the CDR-H3 comprises the amino acid sequence of ARELYYYDX₃RGX₄EEGEGWFDP (SEQ ID NO: 250), wherein X₃ is S or R, and X₄ is A or F; and/or the CDR-L1 comprises the amino acid sequence of TGISSNIGAX₅YDVH (SEQ ID NO: 251), wherein X₅ is G, Q, or E; the CDR-L2 comprises the amino acid sequence of GNSNRPX₆ (SEQ ID NO: 252), wherein X₆ is S or R; and the CDR-L3 comprises the amino acid sequence of QX₇SDRX₈X₉SGSV (SEQ ID NO: 253), wherein X₇ is S or V, and X₈ is S or Y, and X₉ is L or V.

In some embodiments, the CDR-H1 comprises the amino acid sequence of SEQ ID NO: 133, the CDR-H2 comprises the amino acid sequence of SEQ ID NO: 134, the CDR-H3 comprises the amino acid sequence of SEQ ID NO: 135; and/or the CDR-L1 comprises the amino acid sequence of SEQ ID NO: 136, the CDR-L2 comprises the amino acid sequence of SEQ ID NO: 137, and the CDR-L3 comprises the amino acid sequence of SEQ ID NO: 138.

In some embodiments, the VH comprises the amino acid sequence of SEQ ID NO: 167, and the VL comprises the amino acid sequence of SEQ ID NO: 168.

In some embodiments, the VH comprises the amino acid sequence of SEQ ID NO: 139, and the VL comprises the amino acid sequence of SEQ ID NO: 140.

In some embodiments, the CDR-H1 comprises the amino acid sequence of SEQ ID NO: 169, the CDR-H2 comprises the amino acid sequence of SEQ ID NO: 134, the CDR-H3 comprises the amino acid sequence of SEQ ID NO: 170; and/or the CDR-L1 comprises the amino acid sequence of SEQ ID NO: 136, the CDR-L2 comprises the amino acid sequence of SEQ ID NO: 137, and the CDR-L3 comprises the amino acid sequence of SEQ ID NO: 138.

In some embodiments, the VH comprises the amino acid sequence of SEQ ID NO: 171, and the VL comprises the amino acid sequence of SEQ ID NO: 168.

In some embodiments, the CDR-H1 comprises the amino acid sequence of SEQ ID NO: 133, the CDR-H2 comprises the amino acid sequence of SEQ ID NO: 134, the CDR-H3 comprises the amino acid sequence of SEQ ID NO: 135; and/or the CDR-L1 comprises the amino acid sequence of SEQ ID NO: 172, the CDR-L2 comprises the amino acid sequence of SEQ ID NO: 173, and the CDR-L3 comprises the amino acid sequence of SEQ ID NO: 174.

In some embodiments, the VH comprises the amino acid sequence of SEQ ID NO: 167, and the VL comprises the amino acid sequence of SEQ ID NO: 175.

In some embodiments, the CDR-H1 comprises the amino acid sequence of SEQ ID NO: 169, the CDR-H2 comprises the amino acid sequence of SEQ ID NO: 176, the CDR-H3 comprises the amino acid sequence of SEQ ID NO: 177; and/or the CDR-L1 comprises the amino acid sequence of SEQ ID NO: 178, the CDR-L2 comprises the amino acid sequence of SEQ ID NO: 173, and the CDR-L3 comprises the amino acid sequence of SEQ ID NO: 179.

In some embodiments, the VH comprises the amino acid sequence of SEQ ID NO: 180, and the VL comprises the amino acid sequence of SEQ ID NO: 181.

In some aspects, this disclosure provides an antibody that binds to a SARS-CoV-2 spike protein, wherein the antibody comprises: a VH comprising a CDR-H1 comprising the amino acid sequence of X₁TFSSX₂DMS (SEQ ID NO: 254), wherein X₁ is F, W or P, and X₂ is Y, T or S; a CDR-H2 comprising the amino acid sequence of GISGX₃GAITYYTDSVKG (SEQ ID NO: 255), wherein X₃ is S, T, D or E; a CDR-H3 comprising the amino acid sequence of TKENNRX₄TFFDY (SEQ ID NO: 256), wherein X₄ is N, Q, A, L, I, V, or G; and/or a VL comprising a CDR-L1 comprising the amino acid sequence of X₅ASQTVRSDLA (SEQ ID NO: 257), wherein X₅ is R, K, S or T; a CDR-L2 comprising the amino acid sequence of GX₆STRAT (SEQ ID NO: 258), wherein X₆ is A, V, L, I or G; and a CDR-L3 comprising the amino acid sequence of QQYYEWPPHSDS (SEQ ID NO: 186).

In some embodiments, the CDR-H1 comprises the amino acid sequence of X₁TFSSX₂DMS (SEQ ID NO: 259), wherein X₁ is F or P, and X₂ is Y or T; the CDR-H2 comprises the amino acid sequence of GISGX₃GAITYYTDSVKG (SEQ ID NO: 260), wherein X₃ is S or E; the CDR-H3 comprises the amino acid sequence of TKENNRX₄TFFDY (SEQ ID NO: 261), wherein X₄ is N, A, or G; and/or the CDR-L1 comprises the amino acid sequence of X₅ASQTVRSDLA (SEQ ID NO: 262), wherein X₅ is R or S; the CDR-L2 comprises the amino acid sequence of GX₆STRAT (SEQ ID NO: 263), wherein X₆ is A or G; and the CDR-L3 comprises the amino acid sequence of QQYYEWPPHSDS (SEQ ID NO: 186).

In some embodiments, the CDR-H1 comprises the amino acid sequence of SEQ ID NO: 182, the CDR-H2 comprises the amino acid sequence of SEQ ID NO: 183, the CDR-H3 comprises the amino acid sequence of SEQ ID NO: 184; and/or the CDR-L1 comprises the amino acid sequence of SEQ ID NO: 185, the CDR-L2 comprises the amino acid sequence of SEQ ID NO: 77, and the CDR-L3 comprises the amino acid sequence of SEQ ID NO: 186.

In some embodiments, the VH comprises the amino acid sequence of SEQ ID NO: 187, and the VL comprises the amino acid sequence of SEQ ID NO: 188.

In some embodiments, the CDR-H1 comprises the amino acid sequence of SEQ ID NO: 189, the CDR-H2 comprises the amino acid sequence of SEQ ID NO: 183, the CDR-H3 comprises the amino acid sequence of SEQ ID NO: 190; and/or the CDR-L1 comprises the amino acid sequence of SEQ ID NO: 185, the CDR-L2 comprises the amino acid sequence of SEQ ID NO: 77, and the CDR-L3 comprises the amino acid sequence of SEQ ID NO: 186.

In some embodiments, the VH comprises the amino acid sequence of SEQ ID NO: 191, and the VL comprising the amino acid sequence of SEQ ID NO: 188.

In some embodiments, the CDR-H1 comprises the amino acid sequence of SEQ ID NO: 192, the CDR-H2 comprises the amino acid sequence of SEQ ID NO: 183, the CDR-H3 comprises the amino acid sequence of SEQ ID NO: 193; and/or the CDR-L1 comprises the amino acid sequence of SEQ ID NO: 194, the CDR-L2 comprises the amino acid sequence of SEQ ID NO: 195, and the CDR-L3 comprises the amino acid sequence of SEQ ID NO: 186.

In some embodiments, the VH comprises the amino acid sequence of SEQ ID NO: 196, and the VL comprises the amino acid sequence of SEQ ID NO: 197.

In some embodiments, the CDR-H1 comprises the amino acid sequence of SEQ ID NO: 182, the CDR-H2 comprises the amino acid sequence of SEQ ID NO: 198, the CDR-H3 comprises the amino acid sequence of SEQ ID NO: 190; and/or the CDR-L1 comprises the amino acid sequence of SEQ ID NO: 185, the CDR-L2 comprises the amino acid sequence of SEQ ID NO: 195, and the CDR-L3 comprises the amino acid sequence of SEQ ID NO: 186.

In some embodiments, the VH comprises the amino acid sequence of SEQ ID NO: 199, and the VL comprises the amino acid sequence of SEQ ID NO: 200.

In some embodiments, the antibody comprises: a VH comprising a CDR-H1 comprising the amino acid sequence of FTX₁DDYAMH (SEQ ID NO: 264), wherein X₁ is F, W, I, or L; a CDR-H2 comprising the amino acid sequence of GX₂SWNX₃GTIGYADSVKG (SEQ ID NO: 265), wherein X₂ is I, L, V, A, S or T, and X₃ is S, T or G; comprising CDR-H3 comprising the amino acid sequence of X₄KDRKREDPSLGGMDV (SEQ ID NO: 266), wherein X₄ is G I, L, A or V; and/or a VL comprising a CDR-L1 comprising the amino acid sequence of X₅GNNIGSKSVH (SEQ ID NO: 267), wherein X₅ is G, L, I, A or V; a CDR-L2 comprising the amino acid sequence of DDTDRPS (SEQ ID NO: 123); and a CDR-L3 comprising the amino acid sequence of QVWDX₆X₇SDX₈YV (SEQ ID NO: 268), wherein X₆ is S, T, I, L, A or V, and X₇ is S, T, D or E, and X₈ is H, S, T, E or D.

In some embodiments, the CDR-H1 comprises the amino acid sequence of FTX₁DDYAMH (SEQ ID NO: 269), wherein X₁ is F or I; the CDR-H2 comprises the amino acid sequence of GX₂SWNX₃GTIGYADSVKG (SEQ ID NO: 270), wherein X₂ is I or T, and X₃ is S or G; the CDR-H3 comprises the amino acid sequence of X₄KDRKREDPSLGGMDV (SEQ ID NO: 271), wherein X₄ is G or V; and/or the CDR-L1 comprises the amino acid sequence of X₅GNNIGSKSVH (SEQ ID NO: 272), wherein X₅ is G or L; the CDR-L2 comprises the amino acid sequence of DDTDRPS (SEQ ID NO: 123); and the CDR-L3 comprises the amino acid sequence of QVWDX₆X₇SDX₈YV (SEQ ID NO: 273), wherein X₆ is S or V, and X₇ is S or E, and X₈ is H, S, or D.

In some embodiments, the CDR-H1 comprises the amino acid sequence of SEQ ID NO: 119, the CDR-H2 comprises the amino acid sequence of SEQ ID NO: 120, the CDR-H3 comprises the amino acid sequence of SEQ ID NO: 121; and/or the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 124.

In some embodiments, the VH comprises the amino acid sequence of SEQ ID NO: 201, and the VL comprises the amino acid sequence of SEQ ID NO: 202.

In some embodiments, the VH comprises the amino acid sequence of SEQ ID NO: 125, and the VL comprises the amino acid sequence of SEQ ID NO: 126.

In some embodiments, the CDR-H1 comprises the amino acid sequence of SEQ ID NO: 203, the CDR-H2 comprises the amino acid sequence of SEQ ID NO: 204, the CDR-H3 comprises the amino acid sequence of SEQ ID NO: 121; and/or the CDR-L1 comprises the amino acid sequence of SEQ ID NO: 205, the CDR-L2 comprises the amino acid sequence of SEQ ID NO: 123, and the CDR-L3 comprises the amino acid sequence of SEQ ID NO: 206.

In some embodiments, the VH comprises the amino acid sequence of SEQ ID NO: 207, and the VL comprises the amino acid sequence of SEQ ID NO: 208.

In some embodiments, the CDR-H1 comprises the amino acid sequence of SEQ ID NO: 119, the CDR-H2 comprises the amino acid sequence of SEQ ID NO: 209, the CDR-H3 comprises the amino acid sequence of SEQ ID NO: 210; and/or the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 211.

In some embodiments, the VH comprises the amino acid sequence of SEQ ID NO: 212, and the VL comprises the amino acid sequence of SEQ ID NO: 213.

In some embodiments, the CDR-H1 comprises the amino acid sequence of SEQ ID NO: 119, the CDR-H2 comprises the amino acid sequence of SEQ ID NO: 209, the CDR-H3 comprises the amino acid sequence of SEQ ID NO: 210; and/or the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 214.

In some embodiments, the VH comprises the amino acid sequence of SEQ ID NO: 212, and the VL comprises the amino acid sequence of SEQ ID NO: 215.

In some embodiments, the antibody comprises: a VH comprising a CDR-H1 comprising the amino acid sequence of FTFADYX₁MS (SEQ ID NO: 274), wherein X₁ is A, V, S or T; a CDR-H2 comprising the amino acid sequence of FIRSX₂PYGGTTEYAASVKG (SEQ ID NO: 275), wherein X₂ is K, R, E, and D; a CDR-H3 comprising the amino acid sequence of SRDPWYCSGGDCYAVTGSWFDP (SEQ ID NO: 67); and/or a VL comprising a CDR-L1 comprising the amino acid sequence of QASX₃DIKX₄YLN (SEQ ID NO: 276), wherein X₃ is Q, N, K or R and X₄ is K, R. N, or Q; a CDR-L2 comprising the amino acid sequence of DX₅SNLET (SEQ ID NO: 277), wherein X₅ is V, I, L, A, N or Q; and a CDR-L3 comprising the amino acid sequence of QQYX₆X₇LPQT (SEQ ID NO: 278), wherein X₆ is D, E, F or W, and X₇ is N or Q.

In some embodiments, the CDR-H1 comprises the amino acid sequence of FTFADYX₁MS (SEQ ID NO: 279), wherein X₁ is A or T; the CDR-H2 comprises the amino acid sequence of FIRSX₂PYGGTTEYAASVKG (SEQ ID NO: 280), wherein X₂ is K or E; the CDR-H3 comprises the amino acid sequence of SRDPWYCSGGDCYAVTGSWFDP (SEQ ID NO: 67); and/or the CDR-L1 comprises the amino acid sequence of QASX₃DIKX₄YLN (SEQ ID NO: 281), wherein X₃ is Q or R and X₄ is K or Q; the CDR-L2 comprises the amino acid sequence of DX₅SNLET (SEQ ID NO: 282), wherein X₅ is V or Q; and the CDR-L3 comprises the amino acid sequence of QQYX₆X₇LPQT (SEQ ID NO: 283), wherein X₆ is D or W, and X₇ is N or Q.

In some embodiments, the CDR-H1 comprises the amino acid sequence of SEQ ID NO: 65, the CDR-H2 comprises the amino acid sequence of SEQ ID NO: 66, the CDR-H3 comprises the amino acid sequence of SEQ ID NO: 67; and/or the CDR-L1 comprises the amino acid sequence of SEQ ID NO: 68, the CDR-L2 comprises the amino acid sequence of SEQ ID NO: 69, and the CDR-L3 comprises the amino acid sequence of SEQ ID NO: 70.

In some embodiments, the VH comprises the amino acid sequence of SEQ ID NO: 71, and the VL comprises the amino acid sequence of SEQ ID NO: 216.

In some embodiments, the VH comprises the amino acid sequence of SEQ ID NO: 71, and the VL comprises the amino acid sequence of SEQ ID NO: 72.

In some embodiments, the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or the CDR-L1 comprises the amino acid sequence of SEQ ID NO: 68, the CDR-L2 comprises the amino acid sequence of SEQ ID NO: 69, and the CDR-L3 comprises the amino acid sequence of SEQ ID NO: 70.

In some embodiments, the VH comprises the amino acid sequence of SEQ ID NO: 219, and the VL comprises the amino acid sequence of SEQ ID NO: 216.

In some embodiments, the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or the CDR-L1 comprises the amino acid sequence of SEQ ID NO: 220, the CDR-L2 comprises the amino acid sequence of SEQ ID NO: 221, and the CDR-L3 comprises the amino acid sequence of SEQ ID NO: 222.

In some embodiments, the VH comprises the amino acid sequence of SEQ ID NO: 219, and the VL comprises the amino acid sequence of SEQ ID NO: 223.

In some embodiments, the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or the CDR-L1 comprises the amino acid sequence of SEQ ID NO: 224, the CDR-L2 comprises the amino acid sequence of SEQ ID NO: 69, and the CDR-L3 comprises the amino acid sequence of SEQ ID NO: 225.

In some embodiments, the VH comprises the amino acid sequence of SEQ ID NO: 219, and the VL comprises the amino acid sequence of SEQ ID NO: 226.

In some aspects, this disclosure provides an antibody that binds to a SARS-CoV-2 spike protein, the antibody comprising: a VH comprising a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3; and/or a VL comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6; a VH comprising a CDR-H1 comprising the amino acid sequence of SEQ ID NO: 9, a CDR-H2 comprising the amino acid sequence of SEQ ID NO: 10, a CDR-H3 comprising the amino acid sequence of SEQ ID NO: 11; and/or a VL comprising a CDR-L1 comprising the amino acid sequence of SEQ ID NO: 12, a CDR-L2 comprising the amino acid sequence of SEQ ID NO: 13, and a CDR-L3 comprising the amino acid sequence of SEQ ID NO: 14; a VH comprising a CDR-H1 comprising the amino acid sequence of SEQ ID NO: 17, a CDR-H2 comprising the amino acid sequence of SEQ ID NO: 18, a CDR-H3 comprising the amino acid sequence of SEQ ID NO: 19; and/or a VL comprising a CDR-L1 comprising the amino acid sequence of SEQ ID NO: 20, a CDR-L2 comprising the amino acid sequence of SEQ ID NO: 21, and a CDR-L3 comprising the amino acid sequence of SEQ ID NO: 22; a VH comprising a CDR-H1 comprising the amino acid sequence of SEQ ID NO: 25, a CDR-H2 comprising the amino acid sequence of SEQ ID NO: 26, a CDR-H3 comprising the amino acid sequence of SEQ ID NO: 27; and/or a VL comprising a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30; a VH comprising a CDR-H1 comprises the amino acid sequence of SEQ ID NO: 33, a CDR-H2 comprising the amino acid sequence of SEQ ID NO: 34, a CDR-H3 comprising the amino acid sequence of SEQ ID NO: 35; and/or a VL comprising a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 38; a VH comprising a CDR-H1 comprising the amino acid sequence of SEQ ID NO: 49, a CDR-H2 comprising the amino acid sequence of SEQ ID NO: 50, a CDR-H3 comprising the amino acid sequence of SEQ ID NO: 51; and/or a VL comprising a CDR-L1 comprising the amino acid sequence of SEQ ID NO: 52, a CDR-L2 comprising the amino acid sequence of SEQ ID NO: 53 and a CDR-L3 comprising the amino acid sequence of SEQ ID NO: 54; a VH comprising a CDR-H1 comprising the amino acid sequence of SEQ ID NO: 57, a CDR-H2 comprising the amino acid sequence of SEQ ID NO: 58, a CDR-H3 comprising the amino acid sequence of SEQ ID NO: 59; and/or a VL comprising a CDR-L1 comprising the amino acid sequence of SEQ ID NO: 60, a CDR-L2 comprising the amino acid sequence of SEQ ID NO: 61, and a CDR-L3 comprising the amino acid sequence of SEQ ID NO: 62; a VH comprising a CDR-H1 comprising the amino acid sequence of SEQ ID NO: 73, a CDR-H2 comprising the amino acid sequence of SEQ ID NO: 74, a CDR-H3 comprising the amino acid sequence of SEQ ID NO: 75; and/or a VL comprising a CDR-L1 comprising the amino acid sequence of SEQ ID NO: 76, a CDR-L2 comprising the amino acid sequence of SEQ ID NO: 77, and a CDR-L3 comprising the amino acid sequence of SEQ ID NO: 78; a VH comprising a CDR-H1 comprising the amino acid sequence of SEQ ID NO: 81, a CDR-H2 comprising the amino acid sequence of SEQ ID NO: 82, a CDR-H3 comprising the amino acid sequence of SEQ ID NO: 83; and/or a VL comprising a CDR-L1 comprising the amino acid sequence of SEQ ID NO: 84, a CDR-L2 comprising the amino acid sequence of SEQ ID NO: 37, and a CDR-L3 comprising the amino acid sequence of SEQ ID NO: 85; a VH comprising a CDR-H1 comprising the amino acid sequence of SEQ ID NO: 88, a CDR-H2 comprising the amino acid sequence of SEQ ID NO: 89, a CDR-H3 comprising the amino acid sequence of SEQ ID NO: 90; and/or a VL comprising a CDR-L1 comprising the amino acid sequence of SEQ ID NO: 91, a CDR-L2 comprising the amino acid sequence of SEQ ID NO: 92, and a CDR-L3 comprising the amino acid sequence of SEQ ID NO: 93; a VH comprising a CDR-H1 comprising the amino acid sequence of SEQ ID NO: 96, a CDR-H2 comprising the amino acid sequence of SEQ ID NO: 97, a CDR-H3 comprising the amino acid sequence of SEQ ID NO: 98; and/or a VL comprising a CDR-L1 comprising the amino acid sequence of SEQ ID NO: 99, a CDR-L2 comprising the amino acid sequence of SEQ ID NO: 100, and a CDR-L3 comprising the amino acid sequence of SEQ ID NO: 101; a VH comprising a CDR-H1 comprising the amino acid sequence of SEQ ID NO: 104, a CDR-H2 comprising the amino acid sequence of SEQ ID NO: 105, a CDR-H3 comprising the amino acid sequence of SEQ ID NO: 106; and/or a VL comprising a CDR-L1 comprising the amino acid sequence of SEQ ID NO: 107, a CDR-L2 comprising the amino acid sequence of SEQ ID NO: 108, and a CDR-L3 comprising the amino acid sequence of SEQ ID NO: 109; a VH comprising a CDR-H1 comprising the amino acid sequence of SEQ ID NO: 112, a CDR-H2 comprising the amino acid sequence of SEQ ID NO: 113, a CDR-H3 comprising the amino acid sequence of SEQ ID NO: 114; and/or a VL comprising a CDR-L1 comprising the amino acid sequence of SEQ ID NO: 115, a CDR-L2 comprising the amino acid sequence of SEQ ID NO: 45, and a CDR-L3 comprising the amino acid sequence of SEQ ID NO: 116; a VH comprising a CDR-H1 comprising the amino acid sequence of SEQ ID NO: 127, a CDR-H2 comprising the amino acid sequence of SEQ ID NO: 128, a CDR-H3 comprising the amino acid sequence of SEQ ID NO: 129; and/or a VL comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 130; or a VH comprising a CDR-H1 comprising the amino acid sequence of SEQ ID NO: 141, a CDR-H2 comprising the amino acid sequence of SEQ ID NO: 142, a CDR-H3 comprising the amino acid sequence of SEQ ID NO: 143; and/or a VL comprising a CDR-L1 comprising the amino acid sequence of SEQ ID NO: 144, a CDR-L2 comprising the amino acid sequence of SEQ ID NO: 145, and a CDR-L3 comprising the amino acid sequence of SEQ ID NO: 146.

In some embodiments, the CDR-H1 comprises the amino acid sequence of SEQ ID NO: 1, the CDR-H2 comprises the amino acid sequence of SEQ ID NO: 2, and the CDR-H3 comprises the amino acid sequence of SEQ ID NO: 3; and/or the CDR-L1 comprises the amino acid sequence of SEQ ID NO: 4, the CDR-L2 comprises the amino acid sequence of SEQ ID NO: 5, and the CDR-L3 comprises the amino acid sequence of SEQ ID NO: 6.

In some embodiments, the VH comprises the amino acid sequence of SEQ ID NO: 7, and the VL comprises the amino acid sequence of SEQ ID NO: 8.

In some embodiments, the CDR-H1 comprises the amino acid sequence of SEQ ID NO: 9, the CDR-H2 comprises the amino acid sequence of SEQ ID NO: 10, and the CDR-H3 comprises the amino acid sequence of SEQ ID NO: 11; and/or the CDR-L1 comprises the amino acid sequence of SEQ ID NO: 12, the CDR-L2 comprises the amino acid sequence of SEQ ID NO: 13, and the CDR-L3 comprises the amino acid sequence of SEQ ID NO: 14.

In some embodiments, the VH comprises the amino acid sequence of SEQ ID NO: 15, and the VL comprises the amino acid sequence of SEQ ID NO: 16.

In some embodiments, the CDR-H1 comprises the amino acid sequence of SEQ ID NO: 17, the CDR-H2 comprises the amino acid sequence of SEQ ID NO: 18, and the CDR-H3 comprises the amino acid sequence of SEQ ID NO: 19; and/or the CDR-L1 comprises the amino acid sequence of SEQ ID NO: 20, the CDR-L2 comprises the amino acid sequence of SEQ ID NO: 21, and the CDR-L3 comprises the amino acid sequence of SEQ ID NO: 22.

In some embodiments, the VH comprises the amino acid sequence of SEQ ID NO: 23, and the VL comprises the amino acid sequence of SEQ ID NO: 24.

In some embodiments, the CDR-H1 comprises the amino acid sequence of SEQ ID NO: 25, the CDR-H2 comprises the amino acid sequence of SEQ ID NO: 26, and the CDR-H3 comprises the amino acid sequence of SEQ ID NO: 27; and/or the CDR-L1 comprises the amino acid sequence of SEQ ID NO: 28, the CDR-L2 comprises the amino acid sequence of SEQ ID NO: 29, and the CDR-L3 comprises the amino acid sequence of SEQ ID NO: 30.

In some embodiments, the VH comprises the amino acid sequence of SEQ ID NO: 31, and the VL comprises the amino acid sequence of SEQ ID NO: 32.

In some embodiments, the CDR-H1 comprises the amino acid sequence of SEQ ID NO: 33, the CDR-H2 comprises the amino acid sequence of SEQ ID NO: 34, and the CDR-H3 comprises the amino acid sequence of SEQ ID NO: 35; and/or the CDR-L1 comprises the amino acid sequence of SEQ ID NO: 36, the CDR-L2 comprises the amino acid sequence of SEQ ID NO: 37, and the CDR-L3 comprises the amino acid sequence of SEQ ID NO: 38.

In some embodiments, the VH comprises the amino acid sequence of SEQ ID NO: 39, and the VL comprises the amino acid sequence of SEQ ID NO: 40.

In some embodiments, the CDR-H1 comprises the amino acid sequence of SEQ ID NO: 49, the CDR-H2 comprises the amino acid sequence of SEQ ID NO: 50, and the CDR-H3 comprises the amino acid sequence of SEQ ID NO: 51; and/or the CDR-L1 comprises the amino acid sequence of SEQ ID NO: 52, the CDR-L2 comprises the amino acid sequence of SEQ ID NO: 53 and the CDR-L3 comprises the amino acid sequence of SEQ ID NO: 54.

In some embodiments, the VH comprises the amino acid sequence of SEQ ID NO: 55, and the VL comprises the amino acid sequence of SEQ ID NO: 56.

In some embodiments, the CDR-H1 comprises the amino acid sequence of SEQ ID NO: 57, the CDR-H2 comprises the amino acid sequence of SEQ ID NO: 58, and the CDR-H3 comprises the amino acid sequence of SEQ ID NO: 59; and/or the CDR-L1 comprises the amino acid sequence of SEQ ID NO: 60, the CDR-L2 comprises the amino acid sequence of SEQ ID NO: 61, and the CDR-L3 comprises the amino acid sequence of SEQ ID NO: 62.

In some embodiments, the VH comprises the amino acid sequence of SEQ ID NO: 63, and the VL comprises the amino acid sequence of SEQ ID NO: 64.

In some embodiments, the CDR-H1 comprises the amino acid sequence of SEQ ID NO: 73, the CDR-H2 comprises the amino acid sequence of SEQ ID NO: 74, and the CDR-H3 comprises the amino acid sequence of SEQ ID NO: 75; and/or the CDR-L1 comprises the amino acid sequence of SEQ ID NO: 76, the CDR-L2 comprises the amino acid sequence of SEQ ID NO: 77, and the CDR-L3 comprises the amino acid sequence of SEQ ID NO: 78.

In some embodiments, the VH comprises the amino acid sequence of SEQ ID NO: 79, and the VL comprises the amino acid sequence of SEQ ID NO: 80.

In some embodiments, the CDR-H1 comprises the amino acid sequence of SEQ ID NO: 81, the CDR-H2 comprises the amino acid sequence of SEQ ID NO: 82, and the CDR-H3 comprises the amino acid sequence of SEQ ID NO: 83; and/or the CDR-L1 comprises the amino acid sequence of SEQ ID NO: 84, the CDR-L2 comprises the amino acid sequence of SEQ ID NO: 37, and the CDR-L3 comprises the amino acid sequence of SEQ ID NO: 85.

In some embodiments, the VH comprises the amino acid sequence of SEQ ID NO: 86, and the VL comprises the amino acid sequence of SEQ ID NO: 87.

In some embodiments, the CDR-H1 comprises the amino acid sequence of SEQ ID NO: 88, the CDR-H2 comprises the amino acid sequence of SEQ ID NO: 89, and the CDR-H3 comprises the amino acid sequence of SEQ ID NO: 90; and/or the CDR-L1 comprises the amino acid sequence of SEQ ID NO: 91, the CDR-L2 comprises the amino acid sequence of SEQ ID NO: 92, and the CDR-L3 comprises the amino acid sequence of SEQ ID NO: 93.

In some embodiments, the VH comprises the amino acid sequence of SEQ ID NO: 94, and the VL comprises the amino acid sequence of SEQ ID NO: 95.

In some embodiments, the CDR-H1 comprises the amino acid sequence of SEQ ID NO: 96, the CDR-H2 comprises the amino acid sequence of SEQ ID NO: 97, and the CDR-H3 comprises the amino acid sequence of SEQ ID NO: 98; and/or the CDR-L1 comprises the amino acid sequence of SEQ ID NO: 99, the CDR-L2 comprises the amino acid sequence of SEQ ID NO: 100, and the CDR-L3 comprises the amino acid sequence of SEQ ID NO: 101.

In some embodiments, the VH comprises the amino acid sequence of SEQ ID NO: 102, and the VL comprises the amino acid sequence of SEQ ID NO: 103.

In some embodiments, the CDR-H1 comprises the amino acid sequence of SEQ ID NO: 104, the CDR-H2 comprises the amino acid sequence of SEQ ID NO: 105, and the CDR-H3 comprises the amino acid sequence of SEQ ID NO: 106; and/or the CDR-L1 comprises the amino acid sequence of SEQ ID NO: 107, the CDR-L2 comprises the amino acid sequence of SEQ ID NO: 108, and the CDR-L3 comprises the amino acid sequence of SEQ ID NO: 109.

In some embodiments, the VH comprises the amino acid sequence of SEQ ID NO: 110, and the VL comprises the amino acid sequence of SEQ ID NO: 111.

In some embodiments, the CDR-H1 comprises the amino acid sequence of SEQ ID NO: 112, the CDR-H2 comprises the amino acid sequence of SEQ ID NO: 113, and the CDR-H3 comprises the amino acid sequence of SEQ ID NO: 114; and/or the CDR-L1 comprises the amino acid sequence of SEQ ID NO: 115, the CDR-L2 comprises the amino acid sequence of SEQ ID NO: 45, and the CDR-L3 comprises the amino acid sequence of SEQ ID NO: 116.

In some embodiments, the VH comprises the amino acid sequence of SEQ ID NO: 117, and the VL comprises the amino acid sequence of SEQ ID NO: 118.

In some embodiments, the CDR-H1 comprises the amino acid sequence of SEQ ID NO: 127, the CDR-H2 comprises the amino acid sequence of SEQ ID NO: 128, and the CDR-H3 comprises the amino acid sequence of SEQ ID NO: 129; and/or the CDR-L1 comprises the amino acid sequence of SEQ ID NO: 4, the CDR-L2 comprises the amino acid sequence of SEQ ID NO: 5, and the CDR-L3 comprises the amino acid sequence of SEQ ID NO: 130.

In some embodiments, the VH comprises the amino acid sequence of SEQ ID NO: 131, and the VL comprises the amino acid sequence of SEQ ID NO: 132.

In some embodiments, the CDR-H1 comprises the amino acid sequence of SEQ ID NO: 141, the CDR-H2 comprises the amino acid sequence of SEQ ID NO: 142, and the CDR-H3 comprises the amino acid sequence of SEQ ID NO: 143; and/or the CDR-L1 comprises the amino acid sequence of SEQ ID NO: 144, the CDR-L2 comprises the amino acid sequence of SEQ ID NO: 145, and the CDR-L3 comprises the amino acid sequence of SEQ ID NO: 146.

In some embodiments, the VH comprises the amino acid sequence of SEQ ID NO: 147, and the VL comprises the amino acid sequence of SEQ ID NO: 148.

In some embodiments, the antibody is selected from the group consisting of a full length IgG, a Fab fragment, a F(ab′) fragment, a F(ab′)₂ fragment, an scFv, and Fv.

In some embodiments, the antibody is a full length IgG. In some embodiments, the full length IgG comprises a heavy chain constant region of isotype IgG1, IgG2, IgG3, or IgG4. In some embodiments, the antibody is a Fab.

In some embodiments, the antibody binds to a SARS-CoV-2 spike protein of SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, or SARS-CoV-2 Omicron.

In some embodiments, the antibody binds to a SARS-COV-2 spike protein with a K_{D} between 1.0×10⁻⁸ M and 1.0×10⁻¹⁰ M.

In some embodiments, the antibody is a humanized antibody. In some embodiments, the antibody is a human antibody.

In some aspects, this disclosure provides a pharmaceutical composition comprising any one of the antibodies described herein and a pharmaceutically acceptable excipient.

In some aspects, this disclosure provides a nucleic acid sequence encoding any one of the antibodies described herein.

In some aspects, this disclosure provides a vector comprising a nucleic acid sequence encoding any one of the antibodies described herein.

In some aspects, this disclosure provides a cell comprising any one of the antibodies described herein, a nucleic acid described herein, or the vector described herein.

In some aspects, this disclosure provides a method of treating a subject infected with a coronavirus, the method comprising administering any one of the antibodies described herein, a pharmaceutical composition described herein, a nucleic acid sequence described herein, a vector described herein, or a cell described herein.

In some embodiments, the subject is infected with severe acute respiratory syndrome corona virus 1 (SARS-CoV-1), SARS-CoV-2 or Bat coronavirus WIV16.

In some embodiments, the subject is infected with SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, and/or SARS-CoV-2 Omicron.

In some embodiments, the subject is a human subject.

In some embodiments, the subject has an underlying condition that increases the risk of severe disease or death from SARS-CoV-2 infection.

In some embodiments, the underlying condition is cancer, chronic liver disease, chronic kidney disease, chronic lung disease, cystic fibrosis, dementia, diabetes, disability, heart disease, HIV infection, immunocompromise, obesity, pregnancy, sickle cell disease, thalassemia, smoking, transplant recipient, cerebrovascular disease, substance abuse disorders, or tuberculosis.

In some embodiments, the method further comprises administering an additional therapeutic for treatment of coronavirus.

In some embodiments, the additional therapeutic comprises an antiviral molecule or an antibody.

In some embodiments, the antiviral molecule is nirmatrelvir and ritonavir, remdesivir, or molnupiravir.

### BRIEF DESCRIPTION OF DRAWINGS

FIG. 1 is an exemplary schematic depicting a method for identifying pseudoviral neutralizing monoclonal antibodies (mAbs) that bind to the SARS-Cov-2 spike protein (spike protein). The method involves isolating, modifying and testing antibodies from either (1) human peripheral blood mononuclear cells (PMBCs) of patients recently (e.g., infected at least one month prior to collecting the PMBC cells), infected with SARS-CoV-2 or (2) transgenic animal recently immunized with the SARS-CoV-2 spike protein. Over 250 pseudoviral neutralizing antibodies were generated and tested. The antibodies tested bound to multiple epitopes on the spike protein, as determined by binning studies using FORTEBIO OCTET.

FIG. 2 shows representative epitope binning data and potency in pseudovirus neutralizing antibody (PSV) assay (PBMC mAbs). Control antibodies known to bind to SARS-CoV-2 were tested against the mAbs disclosed herein. Bins are based on control antibodies. Verification that the mAbs recognized the same epitopes as the control antibodies was performed via immunoassay showing the ability of one antibody to block the binding of another antibody to the target antigen (e.g., “binning.”). Dotted data points indicate antibodies that do not compete with human angiotensin-converting enzyme 2 (ACE2), which binds to the SARS-CoV-2 spike protein during viral entry.

FIGS. 3A-3B show results of single point PSV assays from humanized chicken derived scFv-Fc supernatants revealing neutralizers which bind on or outside of the SARS-2 spike protein Receptor Binding Domain (RBD) as well as some which bind to SARS-1 spike protein. Positive clones were reformatted to IgG for further characterization and this included evaluation of cross reactivity to SARS1. FIG. 3A shows the neutralization potency to SARS2 for those clones that demonstrated cross-reactivity to SARS1. A-22, as shown in FIG. 3A, corresponds to A-22 in Table 2. FIG. 3B shows the same as FIG. 3A, but for clones that only bound SARS2.

FIGS. 4A-4F show results of pseudovirus assays for mAbs A-6 (FIG. 4A), A-9 (FIG. 4B), A-16 (FIG. 4C), A-18 (FIG. 4D), A-22 (FIG. 4E), and a control antibody, REGN10933 (FIG. 4F), which is known to target the SARS-CoV-2 spike protein RBD. Antibodies were tested for neutralization of wild type, UK (alpha), South African (beta), Southern California (epsilon) and Brazilian (gamma) SARS-CoV-2 virus strains (See Table 3). Antibodies tested showed potent virus neutralization for all variants tested.

FIG. 5 shows that mAbs A-6, A18, and A-22 show cross neutralization to extended beta coronoviral family members. A-6 and A-14 both neutralize SARS2, SARS1, and related Bat coronavirus WIV16 (see phylogenetic tree inset), indicating the breadth of neutralization and therefore a conserved epitope being bound. In contrast, control antibodies RGN10933 and RGN10987 neutralize SARS2, but did not neutralize SARS1 or WIV16-S. OC43 coronavirus was also tested (data not shown), but no neutralization was observed for this more distant coronavirus.

FIG. 6 shows pseudovirus IC50 values agree well with live viral IC50 assay results.

FIGS. 7A-7B show that affinity maturation (Aff Mat) progeny of A-6 have improved binding affinity to SARS2 and SARS1 (FIG. 7A), and improved PsV neutralization to all tested variants (FIG. 7B). Parental antibody A-6 (square) and affinity matured progeny (circles, and highlighted progeny clones in triangle, inverted triangle, or diamond) are VHH72 competitive and neutralize SARS1, WIV16, and all tested SARS2 variants (Alpha, Beta, Delta, Epsilon, Gamma, and Iota). SARS2 PsV IC50=6 nM.

FIGS. 8A-8B show that affinity matured progeny of A-18 have improved binding affinity to SARS2 and SARS1 (FIG. 8A), and improved PsV neutralization to all tested variants (FIG. 8B). Parental antibody A-18 (square) and affinity matured progeny (circles, and highlighted progeny clones in triangle, inverted triangle, or diamond) are VHH72 competitive, and neutralize SARS1, WIV16, and all tested SARS2 variants. SARS2 PsV IC50=10 nM.

FIGS. 9A-9B show that affinity matured progeny of A-22 have similar binding affinity to SARS2 and SARS1 (FIG. 9A), and slightly improved PsV neutralization to all tested variants (FIG. 9B). Parental antibody A-22 (square) and affinity matured progeny (circles, and highlighted progeny clones in triangle, inverted triangle, or diamond) are VHH72 competitive, and neutralize SARS1, and all tested SARS2 variants. SARS2 PsV IC50=5 nM.

FIGS. 10A-10B show that affinity matured progeny of A-9 have improved binding affinity to SARS2 (FIG. 10A), and improved PsV neutralization to all tested variants (FIG. 10B). Parental antibody A-9 (square) and affinity matured progeny (circles, and highlighted progeny clones in triangle, inverted triangle, or diamond) are RGN10933 competitive and neutralize all tested SARS2 variants. SARS2 PsV IC50=200 pM.

FIGS. 11A-11B show that affinity matured progeny of A-16 have improved binding affinity to SARS2 (FIG. 11A), and improved PsV neutralization to all tested variants (FIG. 11B). Parental antibody A-16 (square) and affinity matured progeny (circles, and highlighted progeny clones in triangle, inverted triangle, or diamond) are RGN10987 competitive and neutralize all tested SARS2 variants. SARS2 PsV IC50=1 nM.

FIG. 12 shows all three parental antibodies that were tested in vivo neutralize virus (mouse infection study). K18-hACE2 mice were immunized with protein preparations of parental clones A-9 (RGN10987 epitope bin), A-16 (RGN10933 epitope bin), A-22 (VHH72 epitope bin) or RGN10933 either the day prior (day −1) or day after (day 1) infection with SARS-CoV-2 (day 0). Mice were dosed with 10 mg/kg, 5 mg/kg, 0.5 mg/kg or 0.1 mg/kg of the antibody of interest. Plaque-forming units (PFU) were measured in broncho-alveolar lavage fluid (BALF) and lung tissue. A-9, A-16, and A-22 all resulted in viral neutralization.

FIG. 13 shows exemplary progeny of A-6 have improved PsV IC50 to WT, Delta, and Omicron variants.

FIG. 14 shows exemplary progeny of A-18 have improved PsV IC50 to WT, Delta, and Omicron variants, similar to A-6.

FIG. 15 shows exemplary progeny of antibody A-22 are higher affinity binders, but not as potent for PsV neutralization as A-6 and A-18.

FIG. 16 shows exemplary progeny of antibody A-16 are the most potent mAbs binding outside the ACE2 epitope (Class 3).

FIG. 17 shows exemplary progeny of antibody A-9 are highly potent Class 1 mAbs and have activity against Omicron.

FIG. 18 shows results of multiple PsV assays confirming the activity of parental antibodies against Omicron. Relative rankings are comparable between Test 1 and Test 2. IC50 values can vary by approximately 10-fold.

### DETAILED DESCRIPTION

One challenge in treating SARS-COV-2 is developing therapeutics that are effective against multiple different variants of the virus. In some aspects, disclosed herein, are antibodies that bind to and neutralize numerous SARS-COV-2 variants, including, but not limited to SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, and SARS-CoV-2 Omicron.

Further aspects of the disclosure, including a description of defined terms, are provided below.

### Definitions

Administering: As used herein, the terms “administering” or “administration” means to provide a therapeutic (e.g., an antibody) to a subject in a manner that is physiologically and/or pharmacologically useful (e.g., to treat a condition in the subject). In some embodiments, an antibody or composition described herein is administered to a subject in need thereof via one or more suitable routes of administration, using one or more of a variety of methods known in the art. The route and/or mode of administration will vary depending upon the desired results. An acceptable route of administration may refer to any administration pathway known in the art which may be taken into consideration by a clinician in conjunction with the intended therapeutic use, such as by parenteral administration which is typically associated with injection at or in communication with the intended site of action (e.g., intravenous administration).

In some embodiments, a composition is administered to the same subject once or on multiple occasions.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, single domain antibodies, diabodies, multispecific antibodies (e.g., bispecific antibodies), and antibody fragments including but not limited to scFv, Fab, and (Fab′)2, so long as they exhibit the desired biological activity (e.g., binding to and/or neutralization of SARS-CoV-2). In some embodiments, an antibody is a polypeptide that includes at least one immunoglobulin variable domain or at least one antigenic determinant, e.g., paratope that specifically binds to an antigen. In some embodiments, an antibody is a full-length antibody. In some embodiments, an antibody is a humanized antibody. However, in some embodiments, an antibody is a Fab fragment, a F(ab′) fragment, a F(ab′)2 fragment, a Fv fragment or a scFv fragment. In some embodiments the antibody is a Fab. In some embodiments, an antibody is a diabody. In some embodiments, an antibody comprises a framework having a human germline sequence. In some embodiments, an antibody is a full length IgG. In some embodiments the full length IgG is selected from the group consisting of IgG1, IgG2, IgG3, or IgG4. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, the VL domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecules are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecules are a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designate the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

Conservative amino acid substitution: As used herein, a “conservative amino acid substitution” refers to an amino acid substitution that does not alter the relative charge or size characteristics of the protein in which the amino acid substitution is made (i.e., an amino acid replacement in a protein that changes a given amino acid to a different amino acid with similar biochemical properties). Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids may include substitutions made amongst amino acids within the following groups: (a) M, I, L, V; (b) F, Y, W; (c) K, R, H; (d) A, G; (e) S, T; (f) Q, N; and (g) E, D.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against severe acute respiratory syndrome corona virus 1 (SARS-CoV-1) and SARS-CoV-2 antigens is capable of binding to and neutralizing the SARS-CoV-1 and SARS-CoV-2 with a similar affinity or avidity. In another example, an antibody may be cross reactive with multiple different SARS-CoV-2 variants (e.g., SARS-CoV-2 Alpha, SARS-CoV-2 Beta, SARS-CoV-2 Delta, SARS-CoV-2 Epsilon, SARS-CoV-2 Gamma, SARS-CoV-2 Kappa, SARS-CoV-2 Iota, and SARS-CoV-2 Omicron.

Effective amount/Therapeutically effective amount: As used in herein, the term “effective amount” means the amount of a drug or agent (e.g., an antibody) that elicits a biological or pharmaceutical response of a tissue, system, animal, or human, for example, which is sought by a researcher or clinician. In addition, the term “therapeutically effective amount” refers to an amount that causes an improved treatment, cure, prevention, or alleviation of a disease, disorder, or side effect, or reduces the rate of progression of the disease or condition, compared to a corresponding subject who did not receive the amount. In some embodiments, a therapeutically effective amount is an amount of antibody needed to neutralize SARS-CoV-2 in a subject (e.g., neutralize at least 10%, neutralize at least 25%, neutralize at least 50%, neutralize at least 75%, neutralize at least 90%, neutralize at least 95%, or neutralize at least 99% of SARS-COV-2 in a subject).

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a chicken, have been grafted onto human framework sequences.

Humanized antibody: The term “humanized antibody” refers to antibodies which comprise heavy and light chain variable region sequences from a non-human species (e.g., a chicken) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

Isolated antibody: An “isolated antibody”, as used herein, is intended to refer to an antibody that is substantially free of other antibodies having different antigenic specificities (e.g., an isolated antibody that specifically binds SARS-CoV-2 spike receptor binding domain (RBD) is substantially free of antibodies that specifically bind antigens other than SARS-CoV-2 spike RBD). An isolated antibody that specifically binds SARS-CoV-2 spike RBD complex may, however, have cross-reactivity to other antigens, such as SARS-CoV-1. Moreover, an isolated antibody may be substantially free of other cellular material and/or chemicals.

Neutralizing antibody: A “neutralizing antibody”, as used herein, refers to an antibody that can eliminate or significantly reduce the virulence of the target virus. Neutralizing antibodies bind to a virus and interfere with its ability to infect a cell.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Subject: As used herein, the term “subject” refers to animals in need of alleviation, prevention and/or treatment of diseases or conditions such as viral infections, preferably mammals, and more preferably humans. Mammals also include, but are not limited to, farm animals, race animals, pets, primates, horses, dogs, cats, mice, and rats. The term includes human subjects who have coronavirus infection or are at risk of coronavirus infection. In the present disclosure, administering an antibody or a pharmaceutical composition disclosed herein to a subject in need thereof means administering an effective amount of the antibody or pharmaceutical composition or product or the like. In some embodiments, the subject is a human infant, child, adolescent, or adult. In some embodiments, the subject is an elderly human.

Treat: As used herein, the terms “treat,” “treating,” or “treatment”, and grammatical variants thereof, have the same meaning as commonly understood by those of ordinary skill in the art. In some embodiments, these terms refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For example, slowing the onset or rate of development of a coronavirus infection (e.g., SARS-CoV-2 infection), reducing or alleviating symptoms associated with it, generating a complete or partial regression of the coronavirus infection, or some combination of any of the above. For the purposes of this disclosure, beneficial or desired clinical results include, but are not limited to, reduction or alleviation of symptoms, diminishment of extent of disease, stabilization (e.g., not worsening) of state of disease, delay or slowing of disease progression, amelioration or palliation of the disease state, and remission (whether partial or total), whether detectable or undetectable. “Treat,” “treating,” or “treatment” can also include prolonging survival relative to expected survival time if not receiving treatment. A subject (e.g., a human) in need of treatment may thus be a subject already afflicted with the disease or disorder in question. The terms “treat,” “treating,” or “treatment” include inhibition or reduction of an increase in severity of a pathological state or symptoms relative to the absence of treatment, and is not necessarily meant to imply complete cessation of the relevant disease or condition.

Vector: As used herein, refers to a nucleic acid delivery vehicle into which polynucleotides can be inserted. When the vector can express the protein encoded by the inserted polynucleotide, the vector is called an expression vector. The vector can be introduced into the host cell through transformation, transduction or transfection, so that the genetic material elements it carries can be expressed in the host cell. Vectors are well known to those skilled in the art, including but not limited to: plasmids; phagemids; artificial chromosomes, such as yeast artificial chromosomes (YAC), bacterial artificial chromosomes (BAC) or artificial chromosomes (PAC) derived from P1; bacteriophages such as lambda Bacteriophage or M13 phage and animal virus etc. Animal viruses that can be used as vectors include, but are not limited to, retroviruses (including lentiviruses), adenoviruses, adeno-associated viruses, herpes viruses (such as herpes simplex virus), poxviruses, baculoviruses, papillomaviruses, and papillary viruses. Polyoma vacuole virus (such as SV40). A vector can contain a variety of elements that control expression, including but not limited to promoter sequences, transcription initiation sequences, enhancer sequences, selection elements, and reporter genes. In addition, the vector may also contain an origin of replication site.

### Coronavirus Antibodies

Provided herein, in some aspects, are antibodies that bind to Coronavirus (e.g., SARS-CoV-2). In some embodiments, the antibodies described herein bind to SARS-CoV-2 and its variants. In some embodiments, the antibodies described herein are cross-reactive with other coronaviruses (e.g., SARS-CoV-1, SARS-CoV-2, MERS-CoV, 229E, NL63, HKU1, OC43, bat coronavirus WIV16, and/or pangolin coronavirus). In some embodiments, the antibodies described herein bind to SARS-CoV-1, SARS-CoV2, and Bat WIV16-S.

In some embodiments, antibodies provided herein bind to a coronavirus spike protein (e.g., SARS-CoV-2 spike protein). In some embodiments, antibodies provided herein bind to the receptor binding domain (RBD) of a coronavirus spike protein (e.g., a SARS-CoV-2 spike protein, RBD corresponds to amino acids 319-541 of Genbank Accession No. YP_009724390.1 (SEQ ID NO: 227)).

In some embodiments, antibodies provided herein bind to spike proteins of a SARS-CoV-2 variant, in which the spike protein comprises mutations compared to the spike protein of a wild-type SARS-CoV-2 strain. In some embodiments, the antibodies bind to spike proteins of SARS-CoV-2 variants. Such SARS-CoV-2 variants include, without limitation, SARS-CoV-2 alpha (spike protein containing N501Y mutation relative to SEQ ID NO: 227), SARS-CoV-2 beta (spike protein containing K417N, E484K, and N501Y mutations relative to SEQ ID NO: 227), SARS-CoV-2 gamma (spike protein containing K417T, E484K, and N501Y mutations relative to SEQ ID NO: 227), SARS-CoV-2 delta (spike protein containing L452R and T478K mutations relative to SEQ ID NO: 227), SARS-CoV-2 epsilon (spike protein containing L452R mutation relative to SEQ ID NO: 227), SARS-CoV-2 kappa (spike protein containing L452R and E484Q mutations relative to SEQ ID NO: 227), SARS-CoV-2 iota (spike protein containing S477N and E484K mutations relative to SEQ ID NO: 227), and SARS-CoV-2 omicron (spike protein containing various mutations relative to SEQ ID NO: 227).

Wild type SARS-CoV-2 Spike protein (YP_009724390.1, SEQ ID NO: 227; RBD underlined)

MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

In some embodiments, binding of an antibody described herein to the RBD of a coronavirus spike protein blocks the spike protein from binding to a receptor (e.g., the ACE2 receptor). In some embodiments, binding of an antibody described herein to the RBD of a coronavirus spike protein does not block the spike protein from binding to a receptor (e.g., the ACE2 receptor). In some embodiments, binding of an antibody described herein to the RBD of a coronavirus spike protein neutralizes the coronavirus.

In some embodiments, antibodies disclosed herein bind to the same or overlapping epitopes in the spike protein as known SARS-CoV-2 antibodies. For example, in some embodiments, antibodies described herein (e.g., antibodies A-6, A-18, A-20, A-20.1, A-20.2, A-20.3, A-21, A-21.1, A-21.2, A-21.3, A-22, A-22.2, A-22.2, and A-22.3 shown in Tables 1 and 2) bind to the same or overlapping epitopes in the spike protein as VHH-72 (e.g., as described in Wrapp et al., Cell, Volume 181, Issue 5, 28 May 2020, Pages 1004-1015.e15) and/or compete with VHH-72 in binding to the spike protein. In some embodiments, antibodies described herein (e.g., antibodies A-9, A-24, A-24.1, A-24.2, and A-24.3 shown in Tables 1 and 2) bind to the same or overlapping epitopes in the spike protein as RGN10933 (e.g., as described in Deshpande et al., Front Immunol. 2021; 12: 691715) and/or compete with RGN10933 in binding to the spike protein. In some embodiments, antibodies described herein (e.g., A16, A-23, A-23.1, A-23.2, and A-23.3 shown in Tables 1 and 2) bind to the same or overlapping epitopes in the spike protein as RGN10987 (e.g., as described in Deshpande et al., Front Immunol. 2021; 12: 691715) and/or compete with RGN10987 in binding to the spike protein.

In some embodiments, the antibodies of the present disclosure comprise a heavy chain variable region (VH) comprising a heavy chain complementary determining region 1 (CDR-H1), a heavy chain complementary determining region 2 (CDR-H2), a heavy chain complementary determining region 3 (CDR-H3), and/or (e.g., and) a light chain variable domain (VL) comprising a light chain complementary determining region 1 (CDR-L1), a light chain complementary determining region 1 (CDR-L2), and a light chain complementary determining region 1 (CDR-L3). In some embodiments, an antibody of the present disclosure is affinity matured, e.g., by introducing amino acid variations into one or more CDRs of a parental antibody. In some embodiments, an affinity matured antibody has enhanced binding activity to the spike protein relative to the parental antibody.

In some embodiments, an antibody described herein comprises a CDR-H1, a CDR-H2, and a CDR-H3 that, collectively, contain no more than 10 amino acid variations (e.g., no more than 10, 9, 8, 7, 6, 5, 4, 3, 2, or 1 amino acid variation) as compared with the CDR-H1, CDR-H2, and CDR-H3 of any one of the antibodies of Table 1 or Table 2. In some embodiments, an antibody described herein comprises a CDR-L1, a CDR-L2, and a CDR-L3 that, collectively, contain no more than 10 amino acid variations (e.g., no more than 10, 9, 8, 7, 6, 5, 4, 3, 2, or 1 amino acid variation) as compared with the CDR-LJ, CDR-L2, and CDR-L3 of any one of the antibodies of Table 1 or Table 2. In some embodiments, an antibody described herein comprises a CDR-H1, a CDR-H2, a CDR-H3, a CDR-L1, a CDR-L2, and a CDR-L3 that, collectively, contain no more than 10 amino acid variations (e.g., no more than 10, 9, 8, 7, 6, 5, 4, 3, 2, or 1 amino acid variation) as compared with the CDR-H1, CDR-H2, CDR-H3, CDR-L1, CDR-L2, and CDR-L3 of any one of the antibodies of Table 1 or Table 2.

In some embodiments, an antibody described herein comprises a CDR-HT, a CDR-H2, and a CDR-H3 that, collectively, have at least 90% (e.g., at least 90%, at least 95%, or at least 97%) sequence identity to the CDR-H1, CDR-H2, and CDR-H3 of any one of the antibodies of Table 1 or Table 2. In some embodiments, an antibody described herein comprises a CDR-L1, a CDR-L2, and a CDR-L3 that, collectively, have at least 90% % (e.g., at least 90%, at least 95%, or at least 97%) sequence identity to the CDR-L1, CDR-L2, and CDR-L3 of any one of the antibodies of Table 1 or Table 2. In some embodiments, an antibody described herein comprises a CDR-H1, a CDR-H2, a CDR-H3, a CDR-L1, a CDR-L2, and a CDR-L3 that, collectively, have at least 90% % (e.g., at least 90%, at least 95%, or at least 97%) sequence identity to the CDR-H1, CDR-H2, CDR-H3, CDR-L1, CDR-L2, and CDR-L3 of any one of the antibodies of Table 1 or Table 2.

In some embodiments, an antibody described herein comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any one of the antibodies of Table 1 or Table 2. Alternatively or in addition, an antibody described herein comprises a VL containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VL of any one of the antibodies of Table 1 or Table 2.

In some embodiments, an antibody described herein comprises a VH comprising an amino acid sequence that is at least 80% (e.g., at least 80%, at least 85%, at least 90%, at least 95%, at least 99%) identical to the VH of any one of the antibodies of Table 1 or Table 2. Alternatively or in addition, an antibody described herein comprises a VL comprising an amino acid sequence that is at least 80% (e.g., at least 80%, at least 85%, at least 90%, at least 95%, at least 99%) identical to the VL of any one of any one of the antibodies of Table 1 or Table 2.

In some embodiments, any of the amino acid variations in the antibody sequences (e.g., CDRs, VH, and/or VL) provided herein may be conservative variations (e.g., as described above). In some embodiments, binding to coronavirus (e.g., SARS-CoV-2 spike protein) of the antibodies containing amino acid variations is maintained (e.g., substantially maintained, for example, at least 50%, at least 60%, at least 70%, at least 80%, at least 90%, at least 95% of the binding of the original antibody from which it is derived). Any method can be used to ascertain whether binding to coronavirus (e.g., SARS-CoV-2 spike protein RBD) is maintained, for example, using binding assays and conditions known in the art.

In some embodiments, an antibody described herein binds to a SARS-CoV-2 spike protein and comprises a VH comprising a CDR-H1 comprising the amino acid sequence of FTFX₁NFAMH (SEQ ID NO: 230), wherein X₁ is S, T, D or E; a heavy chain complementary determining region 2 (CDR-H2) comprising the amino acid sequence of VIX₂YDGINX₃YYADSVKG (SEQ ID NO: 231), wherein X₂ is A, L, I, V, E, or D, and X₃ is K, R, D or E; a heavy chain complementary determining region 3 (CDR-H3) comprising the amino acid sequence of ARAQNYYDRX₄GTLQLDAFDI (SEQ ID NO: 232), wherein X₄ is S, T, Y, H; and/or (e.g., and) a light chain variable domain (VL) comprising a light chain complementary determining region 1 (CDR-L1) comprising the amino acid sequence of X₅ASX₆GIRNDLG (SEQ ID NO: 233), wherein X₅ is R, K, V. L, A, or I and X₆ is Q, N, E or D; a light chain complementary determining region 1 (CDR-L2) comprising the amino acid sequence of X₇ASSLX₅S (SEQ ID NO: 234), wherein X₇ is A, V, L, I, E or D, and X₈ is Q, N, D or E; and a light chain complementary determining region 1 (CDR-L3) comprising the amino acid sequence of LQX₉YNYPLT (SEQ ID NO: 235), wherein X₉ is N, Q, E or D.

In some embodiments, in an antibody described herein, the CDR-H1 comprises the amino acid sequence of FTFX₁NFAMH (SEQ ID NO: 236), wherein X₁ is S or D; the CDR-H2 comprises the amino acid sequence of VIX₂YDGINX₃YYADSVKG (SEQ ID NO: 237), wherein X₂ is L or E, and X₃ is K or D; the CDR-H3 comprises the amino acid sequence of ARAQNYYDRX₄GTLQLDAFDI (SEQ ID NO: 238), wherein X₄ is S, Y, or H; and/or (e.g., and) the CDR-L1 comprises the amino acid sequence of X₅ASX₆GIRNDLG (SEQ ID NO: 239), wherein X₅ is R or V and X₆ is Q or D; the CDR-L2 comprises the amino acid sequence of X₇ASSLX₈S (SEQ ID NO: 240), wherein X₇ is A, E or D, and X₈ is Q or E; and the CDR-L3 comprises the amino acid sequence of LQX₉YNYPLT (SEQ ID NO: 241), wherein X₉ is N or D.

In some embodiments, in an antibody described herein, the CDR-H1 comprises the amino acid sequence of SEQ ID NO: 41, the CDR-H2 comprises the amino acid sequence of SEQ ID NO: 42, the CDR-H3 comprises the amino acid sequence of SEQ ID NO: 43; and/or (e.g., and) the CDR-L1 comprises the amino acid sequence of SEQ ID NO: 44, the CDR-L2 comprises the amino acid sequence of SEQ ID NO: 45, and the CDR-L3 comprises the amino acid sequence of SEQ ID NO: 46.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 149, and the VL comprises an amino acid sequence that is at least 80% (e.g., at least 80%, at least 85%, at least 90%, at least 95%, at least 99%) identical to the amino acid sequence of SEQ ID NO: 150. In some embodiments, in an antibody described herein, the VH comprises the amino acid sequence of SEQ ID NO: 149, and the VL comprises the amino acid sequence of SEQ ID NO: 150.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 47, and the VL comprises an amino acid sequence that is at least 80% (e.g., at least 80%, at least 85%, at least 90%, at least 95%, at least 99%) identical to the amino acid sequence of SEQ ID NO: 48. In some embodiments, in an antibody described herein, the VH comprises the amino acid sequence of SEQ ID NO: 47, and the VL comprises the amino acid sequence of SEQ ID NO: 48.

In some embodiments, in an antibody described herein, the CDR-H1 comprises the amino acid sequence of SEQ ID NO: 41, the CDR-H2 comprises the amino acid sequence of SEQ ID NO: 42, the CDR-H3 comprises the amino acid sequence of SEQ ID NO: 43; and/or (e.g., and) the CDR-L1 comprises the amino acid sequence of SEQ ID NO: 151, the CDR-L2 comprises the amino acid sequence of SEQ ID NO: 152, and the CDR-L3 comprises the amino acid sequence of SEQ ID NO: 46.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 149, and the VL comprises an amino acid sequence that is at least 80% (e.g., at least 80%, at least 85%, at least 90%, at least 95%, at least 99%) identical to the amino acid sequence of SEQ ID NO: 153. In some embodiments, in an antibody described herein, the VH comprises the amino acid sequence of SEQ ID NO: 149, and the VL comprises the amino acid sequence of SEQ ID NO: 153.

In some embodiments, in an antibody described herein, the CDR-H1 comprises the amino acid sequence of SEQ ID NO: 154, the CDR-H2 comprises the amino acid sequence of SEQ ID NO: 155, the CDR-H3 comprises the amino acid sequence of SEQ ID NO: 156; and/or (e.g., and) the CDR-L1 comprises the amino acid sequence of SEQ ID NO: 44, the CDR-L2 comprises the amino acid sequence of SEQ ID NO: 157, and the CDR-L3 comprises the amino acid sequence of SEQ ID NO: 46.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 158, and the VL comprises an amino acid sequence that is at least 80% (e.g., at least 80%, at least 85%, at least 90%, at least 95%, at least 99%) identical to the amino acid sequence of SEQ ID NO: 159. In some embodiments, in an antibody described herein, the VH comprises the amino acid sequence of SEQ ID NO: 158, and the VL comprises the amino acid sequence of SEQ ID NO: 159.

In some embodiments, in an antibody described herein, the CDR-H1 comprises the amino acid sequence of SEQ ID NO: 154, the CDR-H2 comprises the amino acid sequence of SEQ ID NO: 160, the CDR-H3 comprises the amino acid sequence of SEQ ID NO: 161; and/or (e.g., and) the CDR-L1 comprises the amino acid sequence of SEQ ID NO: 162, the CDR-L2 comprises the amino acid sequence of SEQ ID NO: 163, and the CDR-L3 comprises the amino acid sequence of SEQ ID NO: 164.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 165, and the VL comprises an amino acid sequence that is at least 80% (e.g., at least 80%, at least 85%, at least 90%, at least 95%, at least 99%) identical to the amino acid sequence of SEQ ID NO: 166. In some embodiments, in an antibody described herein, the VH comprises the amino acid sequence of SEQ ID NO: 165, and the VL comprises the amino acid sequence of SEQ ID NO: 166.

In some embodiments, an antibody described herein binds to a SARS-CoV-2 spike protein and comprises a VH comprising a CDR-H1 comprising the amino acid sequence of FTFX₁RYWMH (SEQ ID NO: 242), wherein X₁ is S, T, K or R; a CDR-H2 comprising the amino acid sequence of RINSDGSSTTYADSVX₂G (SEQ ID NO: 243), wherein X₂ is K, R, D or E; a CDR-H3 comprising the amino acid sequence of ARELYYYDX₃RGX₄EEGEGWFDP (SEQ ID NO: 244), wherein X₃ is S, T, K and R, and X₄ is A, V, I, L, W or F; and/or (e.g., and) a VL comprising a CDR-L1 comprising the amino acid sequence of TGISSNIGAX₅YDVH (SEQ ID NO: 245), wherein X₅ is G, N, Q, D, or E; a CDR-L2 comprising the amino acid sequence of GNSNRPX₆ (SEQ ID NO: 246), wherein X₆ is S, T, K or R; and a CDR-L3 comprising the amino acid sequence of QX₇SDRX₈X₉SGSV (SEQ ID NO: 247), wherein X₇ is S, T, A, I, L or V, and X₈ is S, T, or Y, and X₉ is L, I, or V.

In some embodiments, in an antibody described herein, the CDR-H1 comprises the amino acid sequence of FTFX₁RYWMH (SEQ ID NO: 248), wherein X₁ is S or R; the CDR-H2 comprises the amino acid sequence of RINSDGSSTTYADSVX₂G (SEQ ID NO: 249), wherein X₂ is K or E; the CDR-H3 comprises the amino acid sequence of ARELYYYDX₃RGX₄EEGEGWFDP (SEQ ID NO: 250), wherein X₃ is S or R, and X₄ is A or F; and/or (e.g., and) the CDR-L1 comprises the amino acid sequence of TGISSNIGAX₅YDVH (SEQ ID NO: 251), wherein X₅ is G, Q, or E; the CDR-L2 comprises the amino acid sequence of GNSNRPX₆ (SEQ ID NO: 252), wherein X₆ is S or R; and the CDR-L3 comprises the amino acid sequence of QX₇SDRX₈X₉SGSV (SEQ ID NO: 253), wherein X₇ is S or V, and X₈ is S or Y, and X₉ is L or V.

In some embodiments, in an antibody described herein, the CDR-H1 comprises the amino acid sequence of SEQ ID NO: 133, the CDR-H2 comprises the amino acid sequence of SEQ ID NO: 134, the CDR-H3 comprises the amino acid sequence of SEQ ID NO: 135; and/or (e.g., and) the CDR-L1 comprises the amino acid sequence of SEQ ID NO: 136, the CDR-L2 comprises the amino acid sequence of SEQ ID NO: 137, and the CDR-L3 comprises the amino acid sequence of SEQ ID NO: 138.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 167, and the VL comprises an amino acid sequence that is at least 80% (e.g., at least 80%, at least 85%, at least 90%, at least 95%, at least 99%) identical to the amino acid sequence of SEQ ID NO: 168. In some embodiments, in an antibody described herein, the VH comprises the amino acid sequence of SEQ ID NO: 167, and the VL comprises the amino acid sequence of SEQ ID NO: 168.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 139, and the VL comprises an amino acid sequence that is at least 80% (e.g., at least 80%, at least 85%, at least 90%, at least 95%, at least 99%) identical to the amino acid sequence of SEQ ID NO: 140. In some embodiments, in an antibody described herein, the VH comprises the amino acid sequence of SEQ ID NO: 139, and the VL comprises the amino acid sequence of SEQ ID NO: 140.

In some embodiments, in an antibody described herein, the CDR-H1 comprises the amino acid sequence of SEQ ID NO: 169, the CDR-H2 comprises the amino acid sequence of SEQ ID NO: 134, the CDR-H3 comprises the amino acid sequence of SEQ ID NO: 170; and/or (e.g., and) the CDR-L1 comprises the amino acid sequence of SEQ ID NO: 136, the CDR-L2 comprises the amino acid sequence of SEQ ID NO: 137, and the CDR-L3 comprises the amino acid sequence of SEQ ID NO: 138.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 171, and the VL comprises an amino acid sequence that is at least 80% (e.g., at least 80%, at least 85%, at least 90%, at least 95%, at least 99%) identical to the amino acid sequence of SEQ ID NO: 168. In some embodiments, in an antibody described herein, the VH comprises the amino acid sequence of SEQ ID NO: 171, and the VL comprises the amino acid sequence of SEQ ID NO: 168.

In some embodiments in an antibody described herein, the CDR-H1 comprises the amino acid sequence of SEQ ID NO: 133, the CDR-H2 comprises the amino acid sequence of SEQ ID NO: 134, the CDR-H3 comprises the amino acid sequence of SEQ ID NO: 135; and/or (e.g., and) the CDR-L1 comprises the amino acid sequence of SEQ ID NO: 172, the CDR-L2 comprises the amino acid sequence of SEQ ID NO: 173, and the CDR-L3 comprises the amino acid sequence of SEQ ID NO: 174.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 167, and the VL comprises an amino acid sequence that is at least 80% (e.g., at least 80%, at least 85%, at least 90%, at least 95%, at least 99%) identical to the amino acid sequence of SEQ ID NO: 175. In some embodiments, in an antibody described herein, the VH comprises the amino acid sequence of SEQ ID NO: 167, and the VL comprises the amino acid sequence of SEQ ID NO: 175.

In some embodiments, in an antibody described herein, the CDR-H1 comprises the amino acid sequence of SEQ ID NO: 169, the CDR-H2 comprises the amino acid sequence of SEQ ID NO: 176, the CDR-H3 comprises the amino acid sequence of SEQ ID NO: 177; and/or (e.g., and) the CDR-L1 comprises the amino acid sequence of SEQ ID NO: 178, the CDR-L2 comprises the amino acid sequence of SEQ ID NO: 173, and the CDR-L3 comprises the amino acid sequence of SEQ ID NO: 179.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 180, and the VL comprises an amino acid sequence that is at least 80% (e.g., at least 80%, at least 85%, at least 90%, at least 95%, at least 99%) identical to the amino acid sequence of SEQ ID NO: 181. In some embodiments, in an antibody described herein, the VH comprises the amino acid sequence of SEQ ID NO: 180, and the VL comprises the amino acid sequence of SEQ ID NO: 181.

In some embodiments, an antibody described herein binds to a SARS-CoV-2 spike protein and comprises a VH comprising a CDR-H1 comprising the amino acid sequence of X₁TFSSX₂DMS (SEQ ID NO: 254), wherein X₁ is F, W or P, and X₂ is Y, T or S; a CDR-H2 comprising the amino acid sequence of GISGX₃GAITYYTDSVKG (SEQ ID NO: 255), wherein X₃ is S, T, D or E; a CDR-H3 comprising the amino acid sequence of TKENNRX₄TFFDY (SEQ ID NO: 256), wherein X₄ is N, Q, A, L, I, V, or G; and/or (e.g., and) a VL comprising a CDR-L1 comprising the amino acid sequence of X₅ASQTVRSDLA (SEQ ID NO: 257), wherein X₅ is R, K, S or T; a CDR-L2 comprising the amino acid sequence of GX₆STRAT (SEQ ID NO: 258), wherein X₆ is A, V, L, I or G; and a CDR-L3 comprising the amino acid sequence of QQYYEWPPHSDS (SEQ ID NO: 186).

In some embodiments, in an antibody described herein, the CDR-H1 comprises the amino acid sequence of X₁TFSSX₂DMS (SEQ ID NO: 259), wherein X₁ is F or P, and X₂ is Y or T; the CDR-H2 comprises the amino acid sequence of GISGX₃GAITYYTDSVKG (SEQ ID NO: 260), wherein X₃ is S or E; the CDR-H3 comprises the amino acid sequence of TKENNRX₄TFFDY (SEQ ID NO: 261), wherein X₄ is N, A, or G; and/or (e.g., and) the CDR-L1 comprises the amino acid sequence of X₅ASQTVRSDLA (SEQ ID NO: 262), wherein X₅ is R or S; the CDR-L2 comprises the amino acid sequence of GX₆STRAT (SEQ ID NO: 263), wherein X₆ is A or G; and the CDR-L3 comprises the amino acid sequence of QQYYEWPPHSDS (SEQ ID NO: 186).

In some embodiments, in an antibody described herein, the CDR-H1 comprises the amino acid sequence of SEQ ID NO: 182, the CDR-H2 comprises the amino acid sequence of SEQ ID NO: 183, the CDR-H3 comprises the amino acid sequence of SEQ ID NO: 184; and/or (e.g., and) the CDR-L1 comprises the amino acid sequence of SEQ ID NO: 185, the CDR-L2 comprises the amino acid sequence of SEQ ID NO: 77, and the CDR-L3 comprises the amino acid sequence of SEQ ID NO: 186.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 187, and the VL comprises an amino acid sequence that is at least 80% (e.g., at least 80%, at least 85%, at least 90%, at least 95%, at least 99%) identical to the amino acid sequence of SEQ ID NO: 188. In some embodiments, in an antibody described herein, the VH comprises the amino acid sequence of SEQ ID NO: 187, and the VL comprises the amino acid sequence of SEQ ID NO: 188.

In some embodiments, in an antibody described herein, the CDR-H1 comprises the amino acid sequence of SEQ ID NO: 189, the CDR-H2 comprises the amino acid sequence of SEQ ID NO: 183, the CDR-H3 comprises the amino acid sequence of SEQ ID NO: 190; and/or (e.g., and) the CDR-L1 comprises the amino acid sequence of SEQ ID NO: 185, the CDR-L2 comprises the amino acid sequence of SEQ ID NO: 77, and the CDR-L3 comprises the amino acid sequence of SEQ ID NO: 186.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 191, and the VL comprises an amino acid sequence that is at least 80% (e.g., at least 80%, at least 85%, at least 90%, at least 95%, at least 99%) identical to the amino acid sequence of SEQ ID NO: 188. In some embodiments, in an antibody described herein, the VH comprises the amino acid sequence of SEQ ID NO: 191, and the VL comprises the amino acid sequence of SEQ ID NO: 188.

In some embodiments, in an antibody described herein, the CDR-H1 comprises the amino acid sequence of SEQ ID NO: 192, the CDR-H2 comprises the amino acid sequence of SEQ ID NO: 183, the CDR-H3 comprises the amino acid sequence of SEQ ID NO: 193; and/or (e.g., and) the CDR-L1 comprises the amino acid sequence of SEQ ID NO: 194, the CDR-L2 comprises the amino acid sequence of SEQ ID NO: 195, and the CDR-L3 comprises the amino acid sequence of SEQ ID NO: 186.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 196, and the VL comprises an amino acid sequence that is at least 80% (e.g., at least 80%, at least 85%, at least 90%, at least 95%, at least 99%) identical to the amino acid sequence of SEQ ID NO: 197. In some embodiments, in an antibody described herein, the VH comprises the amino acid sequence of SEQ ID NO: 196, and the VL comprises the amino acid sequence of SEQ TD NO: 197.

In some embodiments, in an antibody described herein, the CDR-H1 comprises the amino acid sequence of SEQ ID NO: 182, the CDR-H2 comprises the amino acid sequence of SEQ ID NO: 198, the CDR-H3 comprises the amino acid sequence of SEQ ID NO: 190; and/or (e.g., and) the CDR-L1 comprises the amino acid sequence of SEQ ID NO: 185, the CDR-L2 comprises the amino acid sequence of SEQ ID NO: 195, and the CDR-L3 comprises the amino acid sequence of SEQ ID NO: 186.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 199, and the VL comprises an amino acid sequence that is at least 80% (e.g., at least 80%, at least 85%, at least 90%, at least 95%, at least 99%) identical to the amino acid sequence of SEQ ID NO: 200. In some embodiments, in an antibody described herein, the VH comprises the amino acid sequence of SEQ ID NO: 199, and the VL comprises the amino acid sequence of SEQ ID NO: 200.

In some embodiments, an antibody described herein binds to a SARS-CoV-2 spike protein and comprises a VH comprising a CDR-H1 comprising the amino acid sequence of FTX₁DDYAMH (SEQ ID NO: 264), wherein X₁ is F, W, I, or L; a CDR-H2 comprising the amino acid sequence of GX₂SWNX₃GTIGYADSVKG (SEQ ID NO: 265), wherein X₂ is I, L, V, A, S or T, and X₃ is S, T or G; comprising CDR-H3 comprising the amino acid sequence of X₄KDRKREDPSLGGMDV (SEQ ID NO: 266), wherein X₄ is G I, L, A or V; and/or (e.g., and) a VL comprising a CDR-L1 comprising the amino acid sequence of X₅GNNIGSKSVH (SEQ ID NO: 267), wherein X₅ is G, L, I, A or V; a CDR-L2 comprising the amino acid sequence of DDTDRPS (SEQ ID NO: 123); and a CDR-L3 comprising the amino acid sequence of QVWDX₆X₇SDX₈YV (SEQ ID NO: 268), wherein X₆ is S, T. I, L, A or V, and X₇ is S, T, D or E, and X₈ is H, S, T, E or D.

In some embodiments, in an antibody described herein, the CDR-H1 comprises the amino acid sequence of FTX₁DDYAMH (SEQ ID NO: 269), wherein X₁ is F or I; the CDR-H2 comprises the amino acid sequence of GX₂SWNX₃GTIGYADSVKG (SEQ ID NO: 270), wherein X₂ is I or T, and X₃ is S or G; the CDR-H3 comprises the amino acid sequence of X₄KDRKREDPSLGGMDV (SEQ ID NO: 271), wherein X₄ is G or V; and/or (e.g., and) the CDR-L1 comprises the amino acid sequence of X₅GNNIGSKSVH (SEQ ID NO: 272), wherein X₅ is G or L; the CDR-L2 comprises the amino acid sequence of DDTDRPS (SEQ ID NO: 123); and the CDR-L3 comprises the amino acid sequence of QVWDX₆X₇SDX₈YV (SEQ ID NO: 273), wherein X₆ is S or V, and X₇ is S or E, and X₈ is H, S, or D.

In some embodiments, in an antibody described herein, the CDR-H1 comprises the amino acid sequence of SEQ ID NO: 119, the CDR-H2 comprises the amino acid sequence of SEQ ID NO: 120, the CDR-H3 comprises the amino acid sequence of SEQ ID NO: 121; and/or (e.g., and) the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 124.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 201, and the VL comprises an amino acid sequence that is at least 80% (e.g., at least 80%, at least 85%, at least 90%, at least 95%, at least 99%) identical to the amino acid sequence of SEQ ID NO: 202. In some embodiments, in an antibody described herein, the VH comprises the amino acid sequence of SEQ ID NO: 201, and the VL comprises the amino acid sequence of SEQ ID NO: 202.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 125, and the VL comprises an amino acid sequence that is at least 80% (e.g., at least 80%, at least 85%, at least 90%, at least 95%, at least 99%) identical to the amino acid sequence of SEQ ID NO: 126. In some embodiments, in an antibody described herein, the VH comprises the amino acid sequence of SEQ ID NO: 125, and the VL comprises the amino acid sequence of SEQ ID NO: 126.

In some embodiments, in an antibody described herein, the CDR-H1 comprises the amino acid sequence of SEQ ID NO: 203, the CDR-H2 comprises the amino acid sequence of SEQ ID NO: 204, the CDR-H3 comprises the amino acid sequence of SEQ ID NO: 121; and/or (e.g., and) the CDR-L1 comprises the amino acid sequence of SEQ ID NO: 205, the CDR-L2 comprises the amino acid sequence of SEQ ID NO: 123, and the CDR-L3 comprises the amino acid sequence of SEQ ID NO: 206.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 207, and the VL comprises an amino acid sequence that is at least 80% (e.g., at least 80%, at least 85%, at least 90%, at least 95%, at least 99%) identical to the amino acid sequence of SEQ ID NO: 208. In some embodiments, in an antibody described herein, the VH comprises the amino acid sequence of SEQ ID NO: 207, and the VL comprises the amino acid sequence of SEQ ID NO: 208.

In some embodiments, in an antibody described herein, the CDR-H1 comprises the amino acid sequence of SEQ ID NO: 119, the CDR-H2 comprises the amino acid sequence of SEQ ID NO: 209, the CDR-H3 comprises the amino acid sequence of SEQ ID NO: 210; and/or (e.g., and) the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 211.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 212, and the VL comprises an amino acid sequence that is at least 80% (e.g., at least 80%, at least 85%, at least 90%, at least 95%, at least 99%) identical to the amino acid sequence of SEQ ID NO: 213. In some embodiments, in an antibody described herein, the VH comprises the amino acid sequence of SEQ ID NO: 212, and the VL comprises the amino acid sequence of SEQ ID NO: 213.

In some embodiments, in an antibody described herein, the CDR-H1 comprises the amino acid sequence of SEQ ID NO: 119, the CDR-H2 comprises the amino acid sequence of SEQ ID NO: 209, the CDR-H3 comprises the amino acid sequence of SEQ ID NO: 210; and/or (e.g., and) the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 214.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 212, and the VL comprises an amino acid sequence that is at least 80% (e.g., at least 80%, at least 85%, at least 90%, at least 95%, at least 99%) identical to the amino acid sequence of SEQ ID NO: 215. In some embodiments, in an antibody described herein, the VH comprises the amino acid sequence of SEQ ID NO: 212, and the VL comprises the amino acid sequence of SEQ ID NO: 215.

In some embodiments, an antibody described herein binds to a SARS-CoV-2 spike protein and comprises a VH comprising a CDR-H1 comprising the amino acid sequence of FTFADYX₁MS (SEQ ID NO: 274), wherein X₁ is A, V, S or T; a CDR-H2 comprising the amino acid sequence of FIRSX₂PYGGTTEYAASVKG (SEQ ID NO: 275), wherein X₂ is K, R, E, and D; a CDR-H3 comprising the amino acid sequence of SRDPWYCSGGDCYAVTGSWFDP (SEQ ID NO: 67); and/or (e.g., and) a VL comprising a CDR-L1 comprising the amino acid sequence of QASX₃DIKX₄YLN (SEQ ID NO: 276), wherein X₃ is Q, N, K or R and X₄ is K, R. N, or Q; a CDR-L2 comprising the amino acid sequence of DX₅SNLET (SEQ ID NO: 277), wherein X₅ is V, I, L, A, N or Q; and a CDR-L3 comprising the amino acid sequence of QQYX₆X₇LPQT (SEQ ID NO: 278), wherein X₆ is D, E, F or W, and X₇ is N or Q.

In some embodiments, in an antibody described herein, the CDR-H1 comprises the amino acid sequence of FTFADYX₁MS (SEQ ID NO: 279), wherein X₁ is A or T; the CDR-H2 comprises the amino acid sequence of FIRSX₂PYGGTTEYAASVKG (SEQ ID NO: 280), wherein X₂ is K or E; the CDR-H3 comprises the amino acid sequence of SRDPWYCSGGDCYAVTGSWFDP (SEQ ID NO: 67); and/or (e.g., and) the CDR-L1 comprises the amino acid sequence of QASX₃DIKX₄YLN (SEQ ID NO: 281), wherein X₃ is Q or R and X₄ is K or Q; the CDR-L2 comprises the amino acid sequence of DX₅SNLET (SEQ ID NO: 282), wherein X₅ is V or Q; and the CDR-L3 comprises the amino acid sequence of QQYX₆X₇LPQT (SEQ ID NO: 283), wherein X₆ is D or W, and X₇ is N or Q.

In some embodiments, in an antibody described herein, the CDR-H1 comprises the amino acid sequence of SEQ ID NO: 65, the CDR-H2 comprises the amino acid sequence of SEQ ID NO: 66, the CDR-H3 comprises the amino acid sequence of SEQ ID NO: 67; and/or (e.g., and) the CDR-L1 comprises the amino acid sequence of SEQ ID NO: 68, the CDR-L2 comprises the amino acid sequence of SEQ ID NO: 69, and the CDR-L3 comprises the amino acid sequence of SEQ ID NO: 70.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 71, and the VL comprises an amino acid sequence that is at least 80% (e.g., at least 80%, at least 85%, at least 90%, at least 95%, at least 99%) identical to the amino acid sequence of SEQ ID NO: 216. In some embodiments, in an antibody described herein, the VH comprises the amino acid sequence of SEQ ID NO: 71, and the VL comprises the amino acid sequence of SEQ ID NO: 216.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 71, and the VL comprises an amino acid sequence that is at least 80% (e.g., at least 80%, at least 85%, at least 90%, at least 95%, at least 99%) identical to the amino acid sequence of SEQ ID NO: 72. In some embodiments, in an antibody described herein, the VH comprises the amino acid sequence of SEQ ID NO: 71, and the VL comprises the amino acid sequence of SEQ ID NO: 72.

In some embodiments, in an antibody described herein, the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or (e.g., and) the CDR-L1 comprises the amino acid sequence of SEQ ID NO: 68, the CDR-L2 comprises the amino acid sequence of SEQ ID NO: 69, and the CDR-L3 comprises the amino acid sequence of SEQ ID NO: 70.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 219, and the VL comprises an amino acid sequence that is at least 80% (e.g., at least 80%, at least 85%, at least 90%, at least 95%, at least 99%) identical to the amino acid sequence of SEQ ID NO: 216. In some embodiments, in an antibody described herein, the VH comprises the amino acid sequence of SEQ ID NO: 216, and the VL comprises the amino acid sequence of SEQ ID NO: 216.

In some embodiments, in an antibody described herein, the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or (e.g., and) the CDR-L1 comprises the amino acid sequence of SEQ ID NO: 220, the CDR-L2 comprises the amino acid sequence of SEQ ID NO: 221, and the CDR-L3 comprises the amino acid sequence of SEQ ID NO: 222.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 219, and the VL comprises an amino acid sequence that is at least 80% (e.g., at least 80%, at least 85%, at least 90%, at least 95%, at least 99%) identical to the amino acid sequence of SEQ ID NO: 223. In some embodiments, in an antibody described herein, the VH comprises the amino acid sequence of SEQ ID NO: 219, and the VL comprises the amino acid sequence of SEQ ID NO: 223.

In some embodiments, in an antibody described herein, the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or (e.g., and) the CDR-L1 comprises the amino acid sequence of SEQ ID NO: 224, the CDR-L2 comprises the amino acid sequence of SEQ ID NO: 69, and the CDR-L3 comprises the amino acid sequence of SEQ ID NO: 225.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 219, and the VL comprises an amino acid sequence that is at least 80% (e.g., at least 80%, at least 85%, at least 90%, at least 95%, at least 99%) identical to the amino acid sequence of SEQ ID NO: 226. In some embodiments, in an antibody described herein, the VH comprises the amino acid sequence of SEQ ID NO: 219, and the VL comprises the amino acid sequence of SEQ ID NO: 226.

In some embodiments, an antibody described herein binds to a SARS-CoV-2 spike protein and comprises:
(i) a VH comprising a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3; and/or a VL comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
(ii) a VH comprising a CDR-H1 comprising the amino acid sequence of SEQ ID NO: 9, a CDR-H2 comprising the amino acid sequence of SEQ ID NO: 10, a CDR-H3 comprising the amino acid sequence of SEQ ID NO: 11; and/or a VL comprising a CDR-L1 comprising the amino acid sequence of SEQ ID NO: 12, a CDR-L2 comprising the amino acid sequence of SEQ ID NO: 13, and a CDR-L3 comprising the amino acid sequence of SEQ ID NO: 14;
(iii) a VH comprising a CDR-H1 comprising the amino acid sequence of SEQ ID NO: 17, a CDR-H2 comprising the amino acid sequence of SEQ ID NO: 18, a CDR-H3 comprising the amino acid sequence of SEQ ID NO: 19; and/or a VL comprising a CDR-L1 comprising the amino acid sequence of SEQ ID NO: 20, a CDR-L2 comprising the amino acid sequence of SEQ ID NO: 21, and a CDR-L3 comprising the amino acid sequence of SEQ ID NO: 22;
(iv) a VH comprising a CDR-H1 comprising the amino acid sequence of SEQ ID NO: 25, a CDR-H2 comprising the amino acid sequence of SEQ ID NO: 26, a CDR-H3 comprising the amino acid sequence of SEQ ID NO: 27; and/or a VL comprising a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
(v) a VH comprising a CDR-H1 comprises the amino acid sequence of SEQ ID NO: 33, a CDR-H2 comprising the amino acid sequence of SEQ ID NO: 34, a CDR-H3 comprising the amino acid sequence of SEQ ID NO: 35; and/or a VL comprising a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 38;
(vi) a VH comprising a CDR-H1 comprising the amino acid sequence of SEQ ID NO: 49, a CDR-H2 comprising the amino acid sequence of SEQ ID NO: 50, a CDR-H3 comprising the amino acid sequence of SEQ ID NO: 51; and/or a VL comprising a CDR-L1 comprising the amino acid sequence of SEQ ID NO: 52, a CDR-L2 comprising the amino acid sequence of SEQ ID NO: 53 and a CDR-L3 comprising the amino acid sequence of SEQ ID NO: 54;
(vii) a VH comprising a CDR-H1 comprising the amino acid sequence of SEQ ID NO: 57, a CDR-H2 comprising the amino acid sequence of SEQ ID NO: 58, a CDR-H3 comprising the amino acid sequence of SEQ ID NO: 59; and/or a VL comprising a CDR-L1 comprising the amino acid sequence of SEQ ID NO: 60, a CDR-L2 comprising the amino acid sequence of SEQ ID NO: 61, and a CDR-L3 comprising the amino acid sequence of SEQ ID NO: 62;
(viii) a VH comprising a CDR-H1 comprising the amino acid sequence of SEQ ID NO: 73, a CDR-H2 comprising the amino acid sequence of SEQ ID NO: 74, a CDR-H3 comprising the amino acid sequence of SEQ ID NO: 75; and/or a VL comprising a CDR-L1 comprising the amino acid sequence of SEQ ID NO: 76, a CDR-L2 comprising the amino acid sequence of SEQ ID NO: 77, and a CDR-L3 comprising the amino acid sequence of SEQ ID NO: 78;
(ix) a VH comprising a CDR-H1 comprising the amino acid sequence of SEQ ID NO: 81, a CDR-H2 comprising the amino acid sequence of SEQ ID NO: 82, a CDR-H3 comprising the amino acid sequence of SEQ ID NO: 83; and/or a VL comprising a CDR-L1 comprising the amino acid sequence of SEQ ID NO: 84, a CDR-L2 comprising the amino acid sequence of SEQ ID NO: 37, and a CDR-L3 comprising the amino acid sequence of SEQ ID NO: 85;
(x) a VH comprising a CDR-H1 comprising the amino acid sequence of SEQ ID NO: 88, a CDR-H2 comprising the amino acid sequence of SEQ ID NO: 89, a CDR-H3 comprising the amino acid sequence of SEQ ID NO: 90; and/or a VL comprising a CDR-L1 comprising the amino acid sequence of SEQ ID NO: 91, a CDR-L2 comprising the amino acid sequence of SEQ ID NO: 92, and a CDR-L3 comprising the amino acid sequence of SEQ ID NO: 93;
(xi) a VH comprising a CDR-H1 comprising the amino acid sequence of SEQ ID NO: 96, a CDR-H2 comprising the amino acid sequence of SEQ ID NO: 97, a CDR-H3 comprising the amino acid sequence of SEQ ID NO: 98; and/or a VL comprising a CDR-L1 comprising the amino acid sequence of SEQ ID NO: 99, a CDR-L2 comprising the amino acid sequence of SEQ ID NO: 100, and a CDR-L3 comprising the amino acid sequence of SEQ ID NO: 101;
(xii) a VH comprising a CDR-H1 comprising the amino acid sequence of SEQ ID NO: 104, a CDR-H2 comprising the amino acid sequence of SEQ ID NO: 105, a CDR-H3 comprising the amino acid sequence of SEQ ID NO: 106; and/or a VL comprising a CDR-L1 comprising the amino acid sequence of SEQ ID NO: 107, a CDR-L2 comprising the amino acid sequence of SEQ ID NO: 108, and a CDR-L3 comprising the amino acid sequence of SEQ ID NO: 109;
(xiii) a VH comprising a CDR-H1 comprising the amino acid sequence of SEQ ID NO: 112, a CDR-H2 comprising the amino acid sequence of SEQ ID NO: 113, a CDR-H3 comprising the amino acid sequence of SEQ ID NO: 114; and/or a VL comprising a CDR-L1 comprising the amino acid sequence of SEQ ID NO: 115, a CDR-L2 comprising the amino acid sequence of SEQ ID NO: 45, and a CDR-L3 comprising the amino acid sequence of SEQ ID NO: 116;
(xiv) a VH comprising a CDR-H1 comprising the amino acid sequence of SEQ ID NO: 127, a CDR-H2 comprising the amino acid sequence of SEQ ID NO: 128, a CDR-H3 comprising the amino acid sequence of SEQ ID NO: 129; and/or a VL comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 130; or
(xv) a VH comprising a CDR-H1 comprising the amino acid sequence of SEQ ID NO: 141, a CDR-H2 comprising the amino acid sequence of SEQ ID NO: 142, a CDR-H3 comprising the amino acid sequence of SEQ ID NO: 143; and/or a VL comprising a CDR-L1 comprising the amino acid sequence of SEQ ID NO: 144, a CDR-L2 comprising the amino acid sequence of SEQ ID NO: 145, and a CDR-L3 comprising the amino acid sequence of SEQ ID NO: 146.

In some embodiments, in an antibody described herein, the CDR-H1 comprises the amino acid sequence of SEQ ID NO: 1, the CDR-H2 comprises the amino acid sequence of SEQ ID NO: 2, and the CDR-H3 comprises the amino acid sequence of SEQ ID NO: 3; and/or (e.g., and) the CDR-L1 comprises the amino acid sequence of SEQ ID NO: 4, the CDR-L2 comprises the amino acid sequence of SEQ ID NO: 5, and the CDR-L3 comprises the amino acid sequence of SEQ ID NO: 6.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 7, and the VL comprises an amino acid sequence that is at least 80% (e.g., at least 80%, at least 85%, at least 90%, at least 95%, at least 99%) identical to the amino acid sequence of SEQ ID NO: 8. In some embodiments, in an antibody described herein, the VH comprises the amino acid sequence of SEQ ID NO: 7, and the VL comprises the amino acid sequence of SEQ ID NO: 8.

In some embodiments, in an antibody described herein, the CDR-H1 comprises the amino acid sequence of SEQ ID NO: 9, the CDR-H2 comprises the amino acid sequence of SEQ ID NO: 10, and the CDR-H3 comprises the amino acid sequence of SEQ ID NO: 11; and/or (e.g., and) the CDR-L1 comprises the amino acid sequence of SEQ ID NO: 12, the CDR-L2 comprises the amino acid sequence of SEQ ID NO: 13, and the CDR-L3 comprises the amino acid sequence of SEQ ID NO: 14.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 15, and the VL comprises an amino acid sequence that is at least 80% (e.g., at least 80%, at least 85%, at least 90%, at least 95%, at least 99%) identical to the amino acid sequence of SEQ ID NO: 16. In some embodiments, in an antibody described herein, the VH comprises the amino acid sequence of SEQ ID NO: 15, and the VL comprises the amino acid sequence of SEQ ID NO: 16.

In some embodiments, in an antibody described herein, the CDR-H1 comprises the amino acid sequence of SEQ ID NO: 17, the CDR-H2 comprises the amino acid sequence of SEQ ID NO: 18, and the CDR-H3 comprises the amino acid sequence of SEQ ID NO: 19; and/or (e.g., and) the CDR-L1 comprises the amino acid sequence of SEQ ID NO: 20, the CDR-L2 comprises the amino acid sequence of SEQ ID NO: 21, and the CDR-L3 comprises the amino acid sequence of SEQ ID NO: 22.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 23, and the VL comprises an amino acid sequence that is at least 80% (e.g., at least 80%, at least 85%, at least 90%, at least 95%, at least 99%) identical to the amino acid sequence of SEQ ID NO: 24. In some embodiments, in an antibody described herein, the VH comprises the amino acid sequence of SEQ ID NO: 23, and the VL comprises the amino acid sequence of SEQ ID NO: 24.

In some embodiments, in an antibody described herein, the CDR-H1 comprises the amino acid sequence of SEQ ID NO: 25, the CDR-H2 comprises the amino acid sequence of SEQ ID NO: 26, and the CDR-H3 comprises the amino acid sequence of SEQ ID NO: 27; and/or (e.g., and) the CDR-L1 comprises the amino acid sequence of SEQ ID NO: 28, the CDR-L2 comprises the amino acid sequence of SEQ ID NO: 29, and the CDR-L3 comprises the amino acid sequence of SEQ ID NO: 30.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 31, and the VL comprises an amino acid sequence that is at least 80% (e.g., at least 80%, at least 85%, at least 90%, at least 95%, at least 99%) identical to the amino acid sequence of SEQ ID NO: 32. In some embodiments, in an antibody described herein, the VH comprises the amino acid sequence of SEQ ID NO: 31, and the VL comprises the amino acid sequence of SEQ ID NO: 32.

In some embodiments, in an antibody described herein, the CDR-H1 comprises the amino acid sequence of SEQ ID NO: 33, the CDR-H2 comprises the amino acid sequence of SEQ ID NO: 34, and the CDR-H3 comprises the amino acid sequence of SEQ ID NO: 35; and/or (e.g., and) the CDR-L1 comprises the amino acid sequence of SEQ ID NO: 36, the CDR-L2 comprises the amino acid sequence of SEQ ID NO: 37, and the CDR-L3 comprises the amino acid sequence of SEQ ID NO: 38.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 39, and the VL comprises an amino acid sequence that is at least 80% (e.g., at least 80%, at least 85%, at least 90%, at least 95%, at least 99%) identical to the amino acid sequence of SEQ ID NO: 40. In some embodiments, in an antibody described herein, the VH comprises the amino acid sequence of SEQ ID NO: 39, and the VL comprises the amino acid sequence of SEQ ID NO: 40.

In some embodiments, in an antibody described herein, the CDR-H1 comprises the amino acid sequence of SEQ ID NO: 49, the CDR-H2 comprises the amino acid sequence of SEQ ID NO: 50, and the CDR-H3 comprises the amino acid sequence of SEQ ID NO: 51; and/or (e.g., and) the CDR-L1 comprises the amino acid sequence of SEQ ID NO: 52, the CDR-L2 comprises the amino acid sequence of SEQ ID NO: 53 and the CDR-L3 comprises the amino acid sequence of SEQ ID NO: 54.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 55, and the VL comprises an amino acid sequence that is at least 80% (e.g., at least 80%, at least 85%, at least 90%, at least 95%, at least 99%) identical to the amino acid sequence of SEQ ID NO: 56. In some embodiments, in an antibody described herein, the VH comprises the amino acid sequence of SEQ ID NO: 55, and the VL comprises the amino acid sequence of SEQ ID NO: 56.

In some embodiments, in an antibody described herein, the CDR-H1 comprises the amino acid sequence of SEQ ID NO: 57, the CDR-H2 comprises the amino acid sequence of SEQ ID NO: 58, and the CDR-H3 comprises the amino acid sequence of SEQ ID NO: 59; and/or (e.g., and) the CDR-L1 comprises the amino acid sequence of SEQ ID NO: 60, the CDR-L2 comprises the amino acid sequence of SEQ ID NO: 61, and the CDR-L3 comprises the amino acid sequence of SEQ ID NO: 62.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 63, and the VL comprises an amino acid sequence that is at least 80% (e.g., at least 80%, at least 85%, at least 90%, at least 95%, at least 99%) identical to the amino acid sequence of SEQ ID NO: 64. In some embodiments, in an antibody described herein, the VH comprises the amino acid sequence of SEQ ID NO: 63, and the VL comprises the amino acid sequence of SEQ ID NO: 64.

In some embodiments, in an antibody described herein, the CDR-H1 comprises the amino acid sequence of SEQ ID NO: 73, the CDR-H2 comprises the amino acid sequence of SEQ ID NO: 74, and the CDR-H3 comprises the amino acid sequence of SEQ ID NO: 75; and/or (e.g., and) the CDR-L1 comprises the amino acid sequence of SEQ ID NO: 76, the CDR-L2 comprises the amino acid sequence of SEQ ID NO: 77, and the CDR-L3 comprises the amino acid sequence of SEQ ID NO: 78.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 79, and the VL comprises an amino acid sequence that is at least 80% (e.g., at least 80%, at least 85%, at least 90%, at least 95%, at least 99%) identical to the amino acid sequence of SEQ ID NO: 80. In some embodiments, in an antibody described herein, the VH comprises the amino acid sequence of SEQ ID NO: 79, and the VL comprises the amino acid sequence of SEQ ID NO: 80.

In some embodiments, in an antibody described herein, the CDR-H1 comprises the amino acid sequence of SEQ ID NO: 81, the CDR-H2 comprises the amino acid sequence of SEQ ID NO: 82, and the CDR-H3 comprises the amino acid sequence of SEQ ID NO: 83; and/or (e.g., and) the CDR-L1 comprises the amino acid sequence of SEQ ID NO: 84, the CDR-L2 comprises the amino acid sequence of SEQ ID NO: 37, and the CDR-L3 comprises the amino acid sequence of SEQ ID NO: 85.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 86, and the VL comprises an amino acid sequence that is at least 80% (e.g., at least 80%, at least 85%, at least 90%, at least 95%, at least 99%) identical to the amino acid sequence of SEQ ID NO: 87. In some embodiments, in an antibody described herein, the VH comprises the amino acid sequence of SEQ ID NO: 86, and the VL comprises the amino acid sequence of SEQ ID NO: 87.

In some embodiments, in an antibody described herein, the CDR-H1 comprises the amino acid sequence of SEQ ID NO: 88, the CDR-H2 comprises the amino acid sequence of SEQ ID NO: 89, and the CDR-H3 comprises the amino acid sequence of SEQ ID NO: 90; and/or (e.g., and) the CDR-L1 comprises the amino acid sequence of SEQ ID NO: 91, the CDR-L2 comprises the amino acid sequence of SEQ ID NO: 92, and the CDR-L3 comprises the amino acid sequence of SEQ ID NO: 93.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 94, and the VL comprises an amino acid sequence that is at least 80% (e.g., at least 80%, at least 85%, at least 90%, at least 95%, at least 99%) identical to the amino acid sequence of SEQ ID NO: 95. In some embodiments, in an antibody described herein, the VH comprises the amino acid sequence of SEQ ID NO: 94, and the VL comprises the amino acid sequence of SEQ ID NO: 95.

In some embodiments, in an antibody described herein, the CDR-H1 comprises the amino acid sequence of SEQ ID NO: 96, the CDR-H2 comprises the amino acid sequence of SEQ ID NO: 97, and the CDR-H3 comprises the amino acid sequence of SEQ ID NO: 98; and/or (e.g., and) the CDR-L1 comprises the amino acid sequence of SEQ ID NO: 99, the CDR-L2 comprises the amino acid sequence of SEQ ID NO: 100, and the CDR-L3 comprises the amino acid sequence of SEQ ID NO: 101.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 102, and the VL comprises an amino acid sequence that is at least 80% (e.g., at least 80%, at least 85%, at least 90%, at least 95%, at least 99%) identical to the amino acid sequence of SEQ ID NO: 103. In some embodiments, in an antibody described herein, the VH comprises the amino acid sequence of SEQ ID NO: 102, and the VL comprises the amino acid sequence of SEQ ID NO: 103.

In some embodiments, in an antibody described herein, the CDR-H1 comprises the amino acid sequence of SEQ ID NO: 104, the CDR-H2 comprises the amino acid sequence of SEQ ID NO: 105, and the CDR-H3 comprises the amino acid sequence of SEQ ID NO: 106; and/or (e.g., and) the CDR-L1 comprises the amino acid sequence of SEQ ID NO: 107, the CDR-L2 comprises the amino acid sequence of SEQ ID NO: 108, and the CDR-L3 comprises the amino acid sequence of SEQ ID NO: 109.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 110, and the VL comprises an amino acid sequence that is at least 80% (e.g., at least 80%, at least 85%, at least 90%, at least 95%, at least 99%) identical to the amino acid sequence of SEQ ID NO: 111. In some embodiments, in an antibody described herein, the VH comprises the amino acid sequence of SEQ ID NO: 110, and the VL comprises the amino acid sequence of SEQ ID NO: 111.

In some embodiments, in an antibody described herein, the CDR-H1 comprises the amino acid sequence of SEQ ID NO: 112, the CDR-H2 comprises the amino acid sequence of SEQ ID NO: 113, and the CDR-H3 comprises the amino acid sequence of SEQ ID NO: 114; and/or (e.g., and) the CDR-L1 comprises the amino acid sequence of SEQ ID NO: 115, the CDR-L2 comprises the amino acid sequence of SEQ ID NO: 45, and the CDR-L3 comprises the amino acid sequence of SEQ ID NO: 116.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 117, and the VL comprises an amino acid sequence that is at least 80% (e.g., at least 80%, at least 85%, at least 90%, at least 95%, at least 99%) identical to the amino acid sequence of SEQ ID NO: 118. In some embodiments, in an antibody described herein, the VH comprises the amino acid sequence of SEQ ID NO: 117, and the VL comprises the amino acid sequence of SEQ ID NO: 118.

In some embodiments, in an antibody described herein, the CDR-H1 comprises the amino acid sequence of SEQ ID NO: 127, the CDR-H2 comprises the amino acid sequence of SEQ ID NO: 128, and the CDR-H3 comprises the amino acid sequence of SEQ ID NO: 129; and/or (e.g., and) the CDR-L1 comprises the amino acid sequence of SEQ ID NO: 4, the CDR-L2 comprises the amino acid sequence of SEQ ID NO: 5, and the CDR-L3 comprises the amino acid sequence of SEQ ID NO: 130.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 131, and the VL comprises an amino acid sequence that is at least 80% (e.g., at least 80%, at least 85%, at least 90%, at least 95%, at least 99%) identical to the amino acid sequence of SEQ ID NO: 132. In some embodiments, in an antibody described herein, the VH comprises the amino acid sequence of SEQ ID NO: 131, and the VL comprises the amino acid sequence of SEQ ID NO: 132.

In some embodiments, in an antibody described herein, the CDR-H1 comprises the amino acid sequence of SEQ ID NO: 141, the CDR-H2 comprises the amino acid sequence of SEQ ID NO: 142, and the CDR-H3 comprises the amino acid sequence of SEQ ID NO: 143; and/or (e.g., and) the CDR-L1 comprises the amino acid sequence of SEQ ID NO: 144, the CDR-L2 comprises the amino acid sequence of SEQ ID NO: 145, and the CDR-L3 comprises the amino acid sequence of SEQ ID NO: 146.

In some embodiments, in an antibody described herein, the VH comprises an amino acid sequence that is at least 80% (e.g., at least 80%, at least 85%, at least 90%, at least 95%, at least 99%) identical to the amino acid sequence of SEQ ID NO: 147, and the VL comprises an amino acid sequence that is at least 80% (e.g., at least 80%, at least 85%, at least 90%, at least 95%, at least 99%) identical to the amino acid sequence of SEQ ID NO: 148. In some embodiments, in an antibody described herein, the VH comprises the amino acid sequence of SEQ ID NO: 147, and the VL comprises the amino acid sequence of SEQ ID NO: 148.

In some embodiments, an antibody described herein is a full length IgG, a Fab fragment, a F(ab′) fragment, a F(ab′)₂ fragment, an scFv, or an Fv. In some embodiments, an antibody described herein is a full length IgG. In further embodiments, the full length IgG is selected from the group consisting of IgG1, IgG2, IgG3, or IgG4. In some embodiments, the antibody of the present disclosure is a Fab. In some embodiments the antibody is a humanized antibody. In some embodiments the antibody is a human antibody.

In some embodiments, any one of the antibodies described herein may comprise a heavy chain constant region (CH) or a portion thereof (e.g., CH1, CH2, CH3, or a combination thereof) fused to the heavy chain variable region. The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. A non-limiting example of a human IgG1 constant region is given below:

(SEQ ID NO: 228)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK

In some embodiments, any one of the antibodies described herein may comprise light a light chain constant region (CL) fused to the light chain variable region. The CL can be any CL known in the art. In some examples, the CL is a kappa light chain. In other examples, the CL is a lambda light chain. In some embodiments, the CL is a kappa light chain, the sequence of which is provided below:

(SEQ ID NO: 229)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC

Other antibody heavy and light chain constant regions are well known in the art, e.g., those provided in the IMGT database (imgt.org) or at vbase2.org/vbstat.php., both of which are incorporated by reference herein.

In some embodiments, one, two or more mutations (e.g., amino acid substitutions) are introduced into the Fc region of an antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fe receptor) on the surface of an effector cell. Mutations in the Fe region of an antibody that decrease or increase the affinity of an antibody for an Fc receptor and techniques for introducing such mutations into the Fc receptor or fragment thereof are known to one of skill in the art. Examples of mutations in the Fe receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fe or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fe or hinge-Fc domain fragment) to decrease the half-life of an antibody in vivo. In some embodiments, one, two or more amino acid mutations (i.e., substitutions, insertions or deletions) are introduced into an IgG constant domain, or FcRn-binding fragment thereof (preferably an Fe or hinge-Fc domain fragment) to increase the half-life of an antibody in vivo. In some embodiments, an antibody described herein can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fe region to alter the effector function(s) of an antibody. The effector ligand to which affinity is altered can be, for example, an Fe receptor or the C1 component of complement. This approach is described in further detail in U.S. Pat. Nos. 5,624,821 and 5,648,260. In some embodiments, the deletion or inactivation (through point mutations or other means) of a constant region domain can reduce Fe receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fe region of an antibody described herein to remove potential glycosylation sites on Fe region, which may reduce Fc receptor binding (see, e.g., Shields R L et al., (2001) J Biol Chem 276: 6591-604).

In some embodiments, one or more amino in the constant region of an antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, an antibody provided herein may comprise mutations that confer desirable properties to the antibodies. For example, to avoid potential complications due to Fab-arm exchange, which is known to occur with native IgG4 mAbs, an antibody provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecules are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecules are a branched oligosaccharide or a branched glycan. In some embodiments, the one or more sugar or carbohydrate molecules includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g., a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, antibodies of the present disclosure can bind to a target antigen (e.g., SARS-CoV-2 spike protein) with relatively high affinity, e.g., with a K_{D} less than 10⁻⁷ M, 10⁻⁸ M, 10⁻⁹ M, 10⁻¹⁰ M, 10⁻¹¹ M or lower. In some embodiments, the antibodies of the present disclosure bind to SARS-CoV-2 spike protein with an affinity between 1.0×10⁻⁷ M and 1.0×10⁻¹¹ M. In some embodiments, the antibodies of the present disclosure bind to SARS-CoV-2 spike protein with an affinity between 1.0×10⁻⁸ M and 1.0×10⁻¹¹ M. In some embodiments, the antibodies of the present disclosure bind to SARS-CoV-2 spike protein with an affinity between 1.0×10⁻⁹ M and 1.0×10⁻¹¹ M.

In some embodiments, the antibodies of the present disclosure bind to SARS-CoV-2 Epsilon spike protein with an affinity between 1.0×10⁻⁸ M and 1.0×10⁻¹¹ M. In some embodiments, the antibodies of the present disclosure bind to SARS-CoV-2 Alpha spike protein with an affinity between 1.0×10⁻⁸ M and 1.0×10⁻¹¹ M. In some embodiments, the antibodies of the present disclosure bind to SARS-CoV-2 Beta spike protein with an affinity between 1.0×10⁻⁸ M and 1.0×10⁻¹¹ M. In some embodiments, the antibodies of the present disclosure bind to SARS-CoV-2 Gamma spike protein with an affinity between 1.0×10⁻⁸ M and 1.0×10⁻¹¹ M. In some embodiments, the antibodies of the present disclosure bind to SARS-CoV-2 Kappa spike protein with an affinity between 1.0×10⁻⁸ M and 1.0×10⁻¹¹ M. In some embodiments, the antibodies of the present disclosure bind to SARS-CoV-2 Delta spike protein with an affinity between 1.0×10⁻⁸ M and 1.0×10⁻¹¹ M. In some embodiments, the antibodies of the present disclosure bind to SARS-CoV-2 Iota spike protein with an affinity between 1.0×10⁻⁸ M and 1.0×10⁻¹¹ M. In some embodiments, the antibodies of the present disclosure bind to SARS-CoV-2 Omicron spike protein with an affinity between 1.0×10⁻⁸ M and 1.0×10⁻¹¹ M. In some embodiments, the antibodies of the present disclosure bind to SARS-CoV-1 spike protein with an affinity between 1.0×10⁻⁸ M and 1.0×10⁻¹¹ M. In some embodiments, the antibodies of the present disclosure bind to WIV spike protein with an affinity between 1.0×10⁻⁸ M and 1.0×10⁻¹¹ M.

In some embodiments, an antibody described herein is a coronavirus (e.g., SARS-CoV-2) neutralizing antibody. In some embodiments, the concentration of an antibody required for 50% neutralization of coronavirus, e.g., SARS-CoV-2 virus, (IC50) is as described in Table 7 and Table 8. In some embodiments, the antibody IC50 for SARS-CoV-2 is between 1.0×10⁻⁷ M and 1.0×10⁻¹² M. In some embodiments, the antibody IC50 for SARS-CoV-2 is between 1.0×10⁻⁸ M and 1.0×10⁻¹¹ M. In some embodiments, the antibody IC50 for SARS-CoV2 is less than 1.0×10⁻⁷ M (e.g., less than 1.0×10⁻⁷ M, less than 1.0×10⁻⁸ M, less than 1.0×10⁻⁹ M, less than 1.0×10⁻¹⁰ M or less than 1.0×10⁻¹¹ M.) In some embodiments, the antibody IC50 for SARS-CoV-2 Beta is between 1.0×10⁻⁷ M and 1.0×10⁻¹² M. In some embodiments, the antibody IC50 for SARS-CoV-2 Beta is between 1.0×10⁻⁷ M and 1.0×10⁻¹² M. In some embodiments, the antibody IC50 is an IC50 set forth in Table 7.

In some embodiments, the antibody IC90 for SARS-CoV-2 is between 0.01 μg/mL and 30 μg/mL. In some embodiments, the antibody IC90 for SARS-CoV-2 is between 0.01 μg/mL and 20 μg/mL. In some embodiments, the antibody IC90 for SARS-CoV-2 is between 0.01 μg/mL and 10 μg/mL. In some embodiments, the antibody IC90 for SARS-CoV-2 is between 0.01 μg/mL and 5 μg/mL. In some embodiments, the antibody IC90 for SARS-CoV-2 is between 0.01 μg/mL and 2 μg/mL. In some embodiments, the antibody IC90 for SARS-CoV-2 is between 0.01 μg/mL and 1 μg/mL. In some embodiments, the antibody IC90 for SARS-CoV-2 is between 0.01 μg/mL and 0.5 μg/mL. In some embodiments, the antibody IC90 for SARS-CoV-2 is less than 30 μg/mL (e.g., less than 20 μg/mL, less than 10 μg/mL, less than 5 μg/mL, less than 1 μg/mL, less than 0.5 μg/mL, less than 0.1 μg/mL, or less than 0.05 μg/mL). In some embodiments, the antibody IC90 for SARS-COV-2 is an IC90 in Table 5 or Table 6.

In some embodiments, the antibody IC90 for SARS-CoV-2 wildtype is between 0.01 μg/mL and 4 μg/mL. In some embodiments, the antibody IC90 for SARS-CoV-2 Alpha is between 0.01 μg/mL and 9 μg/mL. In some embodiments, the antibody IC90 for SARS-CoV-2 Beta is between 0.01 μg/mL and 12 μg/mL. In some embodiments, the antibody IC90 for SARS-CoV-2 Epsilon is between 20 μg/mL and 30 μg/mL. In some embodiments, the antibody 1C90 for SARS-CoV-2 Gamma is between 0.2 μg/mL and 1 μg/mL.

In some embodiments, the antibody IC50 for SARS-CoV-2 is between 0.01 μg/mL and 2 g/mL. In some embodiments, the antibody IC50 for SARS-CoV-2 is between 0.01 μg/mL and 1 μg/mL. In some embodiments, the antibody IC50 for SARS-CoV-2 is between 0.01 μg/mL and 0.5 μg/mL. In some embodiments, the antibody IC50 for SARS-CoV-2 is between 0.01 μg/mL and 0.25 μg/mL. In some embodiments, the antibody IC50 for SARS-CoV-2 is between 0.01 μg/mL and 0.1 μg/mL. In some embodiments, the antibody IC50 for SARS-CoV-2 is less than 5 μg/mL (e.g., less than 2 μg/mL, less than 1 μg/mL, less than 0.5 μg/mL, less than 0.1 μg/mL, or less than 0.05 μg/mL). In some embodiments, the antibody IC50 for SARS-COV-2 is an IC50 in Table 5 or Table 6.

In some embodiments, antibody IC50 for SARS-CoV-2 wildtype is between 0.01 μg/mL and 1 μg/mL. In some embodiments, the antibody IC50 for SARS-CoV-2 Alpha is between 0.01 μg/mL and 2 μg/mL. In some embodiments, the antibody IC50 for SARS-CoV-2 Beta is between 0.01 μg/mL and 3 μg/mL. In some embodiments, the antibody IC50 for SARS-CoV-2 Epsilon is between 0.5 μg/mL and 2 μg/mL. In some embodiments, the antibody IC50 for SARS-CoV-2 Gamma is between 0.1 μg/mL and 1 μg/mL.

In some embodiments, the antibody IC90 for SARS-CoV-1 is between 0.01 μg/mL and 0.5 μg/mL. In some embodiments, the antibody IC90 for WIV-6 is between 0.01 μg/mL and 0.5 μg/mL.

In some embodiments, the antibody IC50 for SARS-CoV-1 is between 0.01 μg/mL and 2 μg/mL. In some embodiments, the antibody IC50 for WIV-6 is between 0.01 μg/mL and 1 μg/mL.

### Pharmaceutical Compositions

In some aspects, the present disclosure also provides a composition (e.g., pharmaceutical composition) comprising an antibody provided herein (e.g., as provided in Tables 1 and 2) and a pharmaceutically acceptable excipient. Pharmaceutically acceptable excipients are well known in the art. A pharmaceutically acceptable excipient includes substances such as disintegrants (e.g., agar, algin, calcium carbonate, carboxymethylcellulose, cellulose, clay, colloidal silicon dioxide, croscarmellose sodium, crospovidone, rubber, magnesium silicate, methylcellulose, polar (e.g., one or more of potassium krillin, sodium alginate, low substituted hydroxypropylcellulose, and crosslinked polyvinylpyrrolidone hydroxypropylcellulose, sodium glycolate starch, and starch), binders (e.g., one or more of microcrystalline cellulose, hydroxymethylcellulose, hydroxypropylcellulose, and polyvinylpyrrolidone.), fillers (e.g., calcium carbonate, calcium phosphate, dicalcium phosphate, calcium trisulfate, carboxymethylcellulose calcium, cellulose, dextrate, dextrin, dextrose, fructose, lactitol, lactose, magnesium carbonate, oxidation (one or more of magnesium, matitol, maltodextrin, maltose, sorbitol, starch, sucrose, sugar, and xylitol) and lubricants (e.g., include agar, calcium stearate, ethyl oleate, ethyl laurate, glycerin, glyceryl palmitostearate, hydrogenated vegetable oil, magnesium oxide, magnesium stearate, mannitol, poloxamer, glycol, sodium benzoate, sodium lauryl sulfate, sodium stearyl, sorbitol, stearic acid, talc, and zinc stearate.) used in formulating pharmaceutical products. Pharmaceutically acceptable excipients are safe for administration to humans in accordance with generally established government standards, including those published by the US Food and Drug Administration.

### Nucleic Acid Sequences, Vectors and Cells

In some aspects, the present disclosure provides nucleic acid sequences encoding any one of the antibodies disclosed herein (e.g., the antibodies of Table 1 or Table 2). In some embodiments, the nucleic acid sequence encodes the VH of any one of the antibodies of Table 1 or Table 2. In some embodiments, the nucleic acid sequence encodes the VL of any one of the antibodies of Table 1 or Table 2. In some embodiments, the nucleic acid sequence encodes the VH and VL of any one of the antibodies of Table 1 or Table 2.

In some embodiments, the nucleic acid molecule is a vector. In some embodiments, the nucleic acid molecule is an expression vector (e.g., an expression vector suitable for expression of the protein in mammalian cells such as human cells).

As will be appreciated by those in the art, the nucleic acid compositions will depend on the format of the proteins. In general, a protein described herein is encoded by a single nucleic acid molecule in a single expression vector for production.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from commercial suppliers.

The nucleic acids and/or expression vectors encoding the antibodies disclosed herein are then transformed into any number of different types of host cells known in the art, including mammalian, bacterial, yeast, insect and/or fungal cells.

The antibody compositions described herein are made by culturing host cells comprising the expression vector(s). Once produced, traditional antibody purification steps may be performed, including a Protein A affinity chromatography step and/or an ion exchange chromatography step.

### Preparation of the Coronavirus Antibodies

Antibodies capable of binding coronavirus as described herein can be made by any method known in the art. See, for example, Harlow and Lane, (1998) Antibodies: A Laboratory Manual, Cold Spring Harbor Laboratory, New York.

In some embodiments, the antibodies and/or nucleic acid sequences encoding the antibodies described herein are identified from subjects (e.g., human subjects) who have been infected with a coronavirus (e.g., SARS-CoV-2). In some embodiments, the antibodies described herein are identified by isolating them from convalescent patient blood samples at least 28 days after confirmed SARS-CoV-2 infection and identifying peripheral blood mononuclear cells (PBMCs) expressing IgGs that bind to either recombinant SARS-CoV-2 spike protein or the SARS-CoV-2 spike protein receptor binding domain (RBD) portion of that protein. In some embodiments, IgG variable regions that bind to SARS-CoV-2 spike protein or the RBD are identified and inserted into expression vectors for antibody production.

In other embodiments, the antibodies and/or nucleic acid sequences encoding the antibodies described herein are identified from animals (e.g., chickens or human transgenic IgG chickens) that have been inoculated with the SARS-COV-2 spike protein or a portion thereof. In such embodiments, lymphocytes from immunized animals are screened for binding to either recombinant SARS-CoV-2 spike protein or the receptor binding domain (RBD) portion of that protein using GEM (Gel Encapsulated Microenvironments), and sequencing is performed to determine antibody identity.

The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

Antigen-binding fragments of an intact antibody (full-length antibody) can be prepared via routine methods. For example, F(ab′)₂ fragments can be produced by pepsin digestion of an antibody molecule, and Fab fragments that can be generated by reducing the disulfide bridges of F(ab′)₂ fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be isolated and sequenced (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv antibodies specific to SARS-CoV-2 can be identified from the library following routine procedures. Positive antibodies can be subjected to further screening to identify those that have high SARS-CoV-2 binding affinity.

In some embodiments, an antibody is prepared by recombinant technology. Nucleic acids encoding the heavy and light chain of a coronavirus antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some embodiments, methods for preparing an antibody described herein involve a recombinant expression vector that encodes both the heavy chain and the light chain of an coronavirus antibody, as also described herein. The recombinant expression vector can be introduced into a suitable host cell by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

Alternatively, each of the expression vectors can be introduced into suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

In some embodiments, standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

### Method of Treatment

In some aspects, the present disclosure provides a method of treating a subject infected with a coronavirus, the method comprising administering to a subject an antibody provided herein (e.g., as provided in Tables 1 and 2). In some embodiments, the method of treating a subject infected with a coronavirus comprises administering any one of the antibodies in Table 2. In some embodiments, the method comprises administering the antibody A-20, A-20.1, A-20.2, or A-20.3 of Table 2. In some embodiments, the method comprises administering the antibody A-21, A-21.1, A-21.2, or A-21.3 of Table 2. In some embodiments, the method comprises administering the antibody A-22, A-22.1, A-22.2, or A-22.3 of Table 2. In some embodiments, the method comprises administering the antibody A-23, A-23.1, A-23.2, or A-23.3 of Table 2. In some embodiments, the method comprises administering the antibody A-24, A-24.1, A-24.2, or A-24.3 of Table 2.

In some embodiments, the method comprises administering a pharmaceutical composition comprising an antibody provided herein and a pharmaceutically acceptable excipient. In some embodiments, the method comprises administering a nucleic acid sequence encoding an antibody provided herein (e.g., as provided in Tables 1 and 2).

In some embodiments, the subject is infected with SARS-CoV-1, SARS-CoV-2 or Bat coronavirus WIV16. In some embodiments, the subject is infected with SARS-CoV-2. In some embodiments, the subject is infected with SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, or SARS-CoV-2 Omicron.

In some embodiments, the subject is a human subject. In some embodiments, the subject has an underlying condition that increases the risk of severe disease or death from SARS-CoV-2 infection. In some embodiments, the underlying condition is cancer, chronic liver disease, chronic kidney disease, chronic lung disease (e.g., asthma, bronchiectasis, bronchopulmonary dysplasia, chronic obstructive pulmonary disease, interstitial lung disease, pulmonary embolism, pulmonary hypertension), cystic fibrosis, dementia or Alzheimer's, diabetes (type 1 or type 2), disability (e.g., people with: any type of disability that makes it more difficult to do certain activities or interact with the world around them, including people who need help with self-care or daily activities, attention-deficit/hyperactivity disorder (ADHD), cerebral palsy, birth defects, intellectual and developmental disabilities, learning disabilities, spinal cord injuries, Down syndrome), heart disease (e.g., heart failure, coronary artery disease, cardiomyopathies, hypertension), HIV infection, immunocompromise, mental health conditions (mood disorders, including depression, and schizophrenia spectrum disorders), overweight (BMI=25-30 kg/m²), obesity (BMI=30-40 kg/m²), severe obesity (BMI>40 kg/m²), pregnancy, sickle cell disease, thalassemia, smoking, transplant recipient, cerebrovascular disease (e.g., stroke), substance abuse disorders (e.g., alcohol, opioid, or cocaine use disorder), or tuberculosis.

In some embodiments, the method further comprises administering an additional therapeutic for treatment of coronavirus. In some embodiments, the method further comprises administering an additional therapeutic for treatment of SARS-CoV-2. In some embodiments, the additional therapeutic is an antiviral molecule or an antibody. In some embodiments, the antiviral molecule is remdesivir (VEKLURY®), ritonavir-boosted nirmatrelvir (PAXLOVID™), or molnupiravir, (LAGEVRIO™).

### Kits for Therapeutic and Diagnostic Applications

In some aspects, the present provides kits for the antibodies disclosed herein. Such kits can include one or more containers comprising an coronavirus antibody, e.g., any of those described herein.

In some embodiments, the kit comprises instructions for use in accordance with any of the methods described herein. The included instructions can comprise a description of administration of the coronavirus antibody to treat, delay the onset, or alleviate a target disease as those described herein. The kit may further comprise a description of selecting an individual suitable for treatment based on identifying whether that individual has the target disease. In some embodiments, the instructions comprise a description of administering an antibody to an individual at risk of the target disease.

The instructions relating to the use of a coronavirus antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder treatable by modulating immune responses, such as autoimmune diseases. Instructions may be provided for practicing any of the methods described herein.

The kits of this invention are in suitable packaging. Suitable packaging includes, but is not limited to, vials, bottles, jars, flexible packaging (e.g., scaled Mylar or plastic bags), and the like.

Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an coronavirus antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting coronavirus in a sample. Such a kit may comprise any of the coronavirus antibodies described herein. In some instances, the coronavirus antibody can be conjugated with a detectable label (e.g., a fluorescent molecule). As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

In some embodiments, the kit comprises a secondary antibody capable of binding to coronavirus antibody. The kit may further comprise instructions for using the antibodies for detecting SARS-CoV-2.

### EXAMPLES

Coronaviruses (CoV) are a large family of viruses that cause a range of illnesses from mild (e.g., the common cold), to severe (e.g., Middle East Respiratory Syndrome (MERS CoV) and Severe Acute Respiratory Syndrome (SARS-CoV)). The COVID-19 (Coronavirus Disease 2019) outbreak, caused by the Severe Acute Respiratory Syndrome Coronavirus 2 (SARS-CoV-2), began in Wuhan, China in December 2019, and quickly became a global pandemic. As of June 2022, COVID-19 has been confirmed in more than 535 million people worldwide, resulting in over 6.3 million deaths.

Monoclonal antibodies (mAbs) that target the SARS-CoV-2 spike protein have demonstrated clinical benefits for COVID-19 treatment. The SARS-CoV-2 spike protein mediates host cell attachment and invasion. The spike protein receptor-binding domain (RBD) binds angiotensin-converting enzyme 2 (ACE2) on a host cell, leading to virus-host cell membrane fusion and viral entry.

However, spike protein mutations in SARS-CoV-2 variants have rendered most currently available SARS-CoV-2 mAbs ineffective. For example, the Omicron (B.1.1.529, BA.1, BA.1.1, BA.2, BA.3, BA.4 and BA.5) variants of concern (VOCs) are currently the dominant VOCs in the United States. However, Omicron has markedly reduced susceptibility to known SARS-CoV-2 mAbs due to numerous mutations in the spike protein. Additionally, SARS-CoV-2 variants with substitutions specific to the spike protein RBD (e.g., from the United Kingdom (B.1.1.7) (alpha), Republic of South Africa (B.1.351) (beta), Brazil (P.1 lineage)(gamma), New York (B.1.526)(iota), and California (B.1.427/B.1.429 or CAL.20C lineage) (epsilon)), have also shown mAb resistance and reduced neutralization in pseudovirus neutralization (PsV) assays.

The mortality and continuing health issues associated with Coronavirus infections, particularly SARS-CoV-2 and its variants, are of great concern worldwide. There remains a need for the development and evaluation of mAbs that effectively protect both against circulating and future SARS-CoV-2 variants and the wild-type SARS-CoV-2 virus, which remains present globally.

The following Examples describe the development of Coronavirus antibodies for the treatment of diseases associated with infection by SARS-CoV-2 and its variants. Human peripheral blood mononuclear cells (PBMCs) and humanized chicken lymphocytes were used to isolate mAbs. Antibodies that effectively neutralized SARS-CoV-2 were further optimized by affinity maturation. The neutralization and binding properties of select antibodies were tested in vitro and in vivo. Select antibodies were effective in neutralizing SARS-CoV-2, SARS-CoV-2 variants, and other Coronavirus family members, including SARS-CoV-1 and WIV.

It should be understood that the following Examples, while indicating preferred embodiments of the invention, are given by way of illustration only. The invention is not limited to the material, proportions, conditions and procedures set forth in the Examples. From the above discussion and these Examples, one skilled in the art can ascertain the essential characteristics of the subject technology, and without departing from the spirit and scope thereof, can make various changes and modifications of the subject technology to adapt it to various uses and conditions.

### Example 1: SARS-CoV-2 Antibodies from Convalescent Patient PBMCs and Transgenic Animals

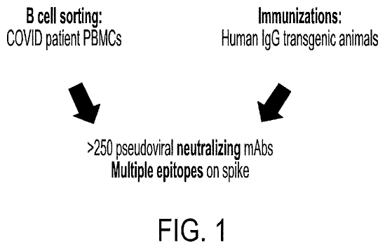

Human antibodies were isolated from convalescent patient blood samples>28 days after confirmed SARS-CoV-2 infection (FIG. 1). PBMCs were sorted by fluorescence-activated cell sorting (FACS) for IgG binding to either recombinant SARS-CoV-2 spike protein or the receptor binding domain (RBD) portion of that protein. Single B cells were sorted into individual wells, lysed, and IgG variable regions were amplified via reverse transcriptase (RT) PCR followed by antibody heavy and light chain variable region PCR amplification using human specific primer sets. A second round of heavy and light chain PCR was performed using primers that also included a 40 bp homology region for homologous recombination cloning together with digested antibody expression vectors into S. cerevisiae. After chemical transformation heavy chain and light chain antibody sequences for individual colonies were obtained and these monoclonal cultures were used to produce and purify antibody for characterization. Positive clones were also reformatted and expressed in mammalian cells for further characterization. Results are shown in Table 11.

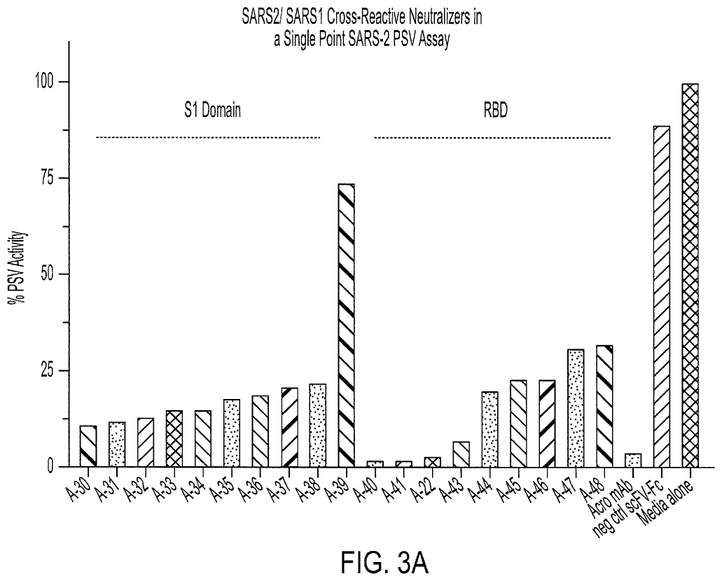

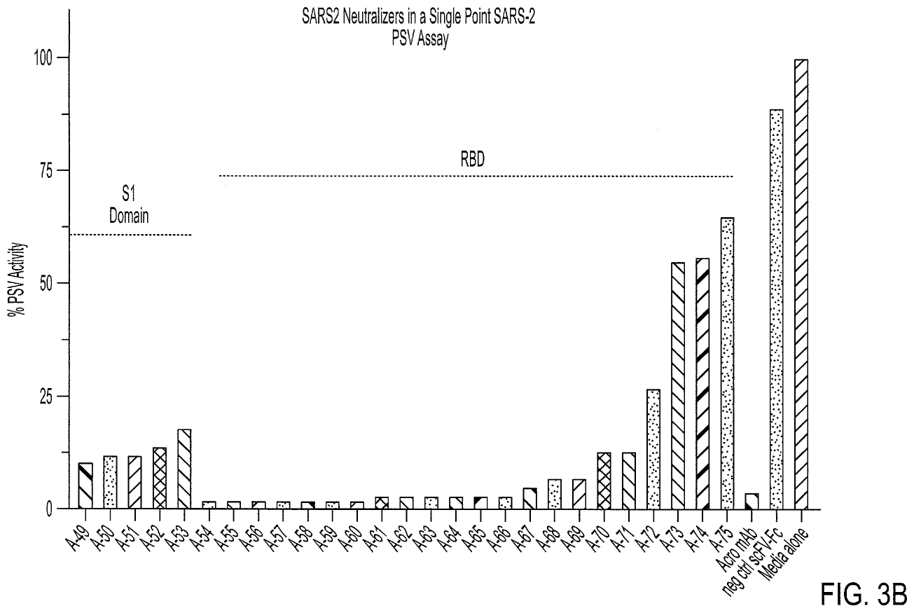

Humanized chickens were immunized with SARS-CoV-2 spike protein (FIG. 1). Lymphocytes from immunized chickens were screened for binding to either recombinant SARS-CoV-2 spike protein or the receptor binding domain (RBD) portion of that protein using GEM (Gel Encapsulated Microenvironments). Lymphocytes in GEM droplet with positive signal were recovered and lysed. IgG variable regions were amplified via reverse transcriptase (RT) PCR followed by antibody heavy and light chain variable region PCR amplification. Human IgG VH and VL domain were cloned into pcDNA 3.4 in scFv-Fc (human IgG1 Fe) format and expressed transiently in mammalian cells. The supernatant was used for binding confirmation and initial characterization (FIGS. 3A-3B). Positive clones were reformatted to IgG for further characterization. Exemplary sequences of the isolated antibodies are shown in Table 1.

### Example 2: Epitope Binning of SARS-CoV-2 Binding Antibodies

Epitope binning of a panel of anti-SARS-CoV-2 RBD antibodies was performed in a LSA instrument (CARTERRA). Following manufacturer instructions, the entire set of antibodies in the panel was crosslinked to a CM200M sensor chip (CARTERRA) at a constant concentration 5 μg/ml in 10 mM Acetate buffer, pH 4.5. In a sequential series of cycles, selected antibodies from this panel were then individually evaluated for the ability to compete for antigen binding against the entire set of immobilized antibodies. To accomplish that, SPR signal was monitored while 40 nM SARS-CoV-2 RBD-mFc was allowed to interact with the chip for 300 seconds and immediately followed by 10 μg/ml of the selected antibody (also called competing antibody) for another 300 seconds. Regeneration using 0.85% phosphoric acid was then used to strip both antigen and competing antibody and the next cycle of antibody competition was run. A total of 62 cycles of competition analyses were carried out. Antigen and competing antibodies were diluted in running buffer (10 mM HEPES, pH 7.4, 150 mM NaCl, 0.05% Tween-20). All incubations were carried out at room temperature (25° C.).

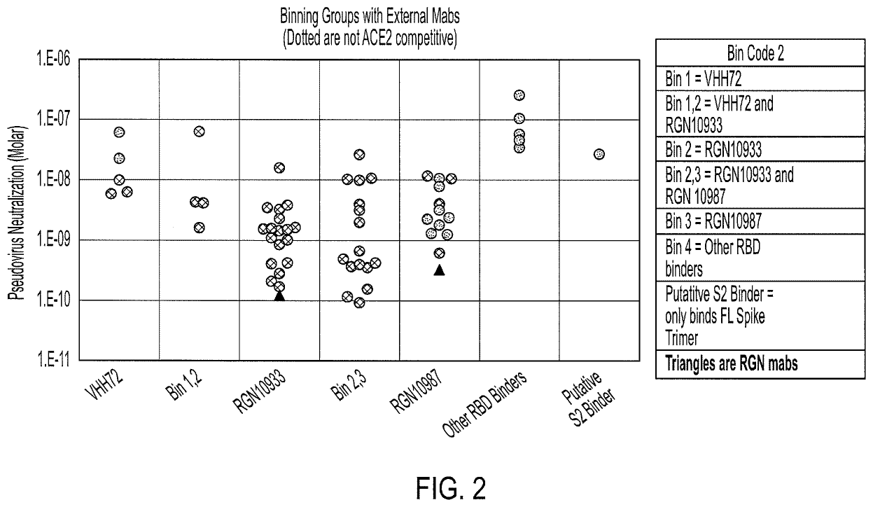

Data analysis was performed using the instrument supplied Epitope software (CARTERRA). Exemplary results of epitope binning data vs PsV assay potency is shown in FIG. 2. Isolated mAbs were tested for competition with VHH72 (a known Class 1/4, SARS-CoV RBD and SARS-CoV-2 RBD mAb), RGN10933 (a known Class 1 SARS-CoV-2 RBD mAb), and RGN10987 (a known Class 3 SARS-CoV-2 RBD mAb). Select antibodies were binned based on competition with the known antibodies (Table 4).

### Example 3: Pseudovirus Neutralization Assays

To identify anti-SARS-CoV-2 antibodies which neutralize SARS-CoV-2 variants, in vitro studies were performed against a panel of reporter virus particles (RVPs) expressing wild-type and variant spike proteins. These RVPs were generated using a luciferase reporter to allow for high-throughput neutralization.

On the day of the assay, antibodies were prepared to a normalized concentration of 500 nM. Each RVP requires 10 μL per well of antibody. Normalized antibodies were then transferred to a fresh V-bottom plate and serially diluted, 1:3, using assay medium (1000 ml DMEM phenol-red free, Gibco; 100 mL HI-FBS, Sigma; 5 mL L-Glutamine, Gibco; 5 mL Sodium Pyruvate, Gibco). RVPs were added to a 384-well white plate (Corning) at 10 μl per well, except for cell control wells which received assay media. The serially diluted antibodies were then transferred at 10 μL per well to the plate with RVPs. The RVPs with antibodies were incubated at 37° C. for 1 hour.

During incubation, HEK 293T cells over-expressing ACE2 were washed with PBS and VERSENE was added and incubated at 37° C. for ~5 minutes until the cells dislodged. After the 1-hour incubation, 9000 cells per well in assay medium were added to the RVP/antibody. The plates were then incubated at 37° C., 5% CO₂ for 72 hours.

On day 3, RENILLA-GLO (Promega) was prepared according to vendor's instruction. The supernatant was removed from the cells and were washed with 50 μL of PBS, twice. After the final wash, 10 μL of RENILLA-GLO was added to the cells and incubated at room temperature for 10 minutes in the dark. Plates were read on a PHERASTAR plate reader using a luminescence module.

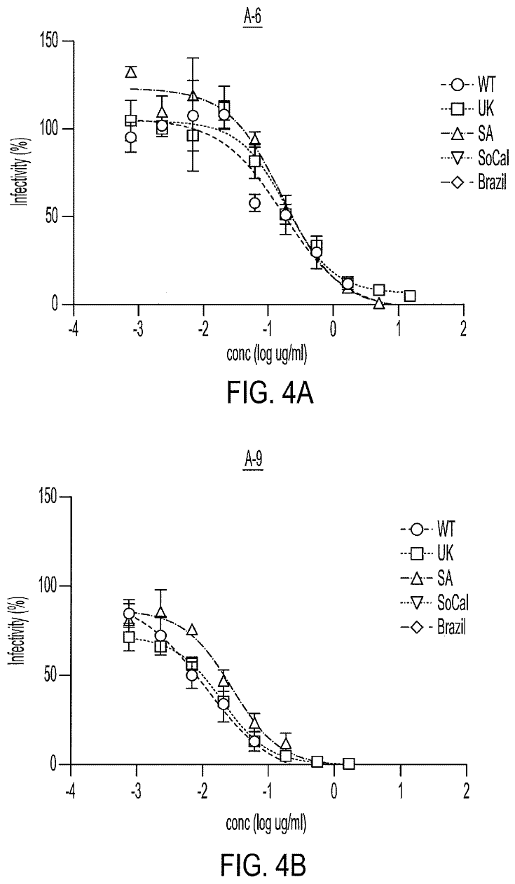

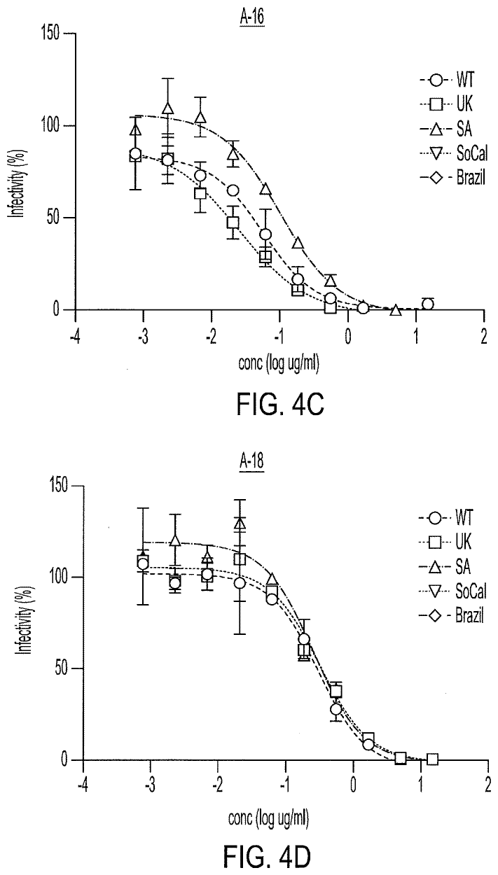

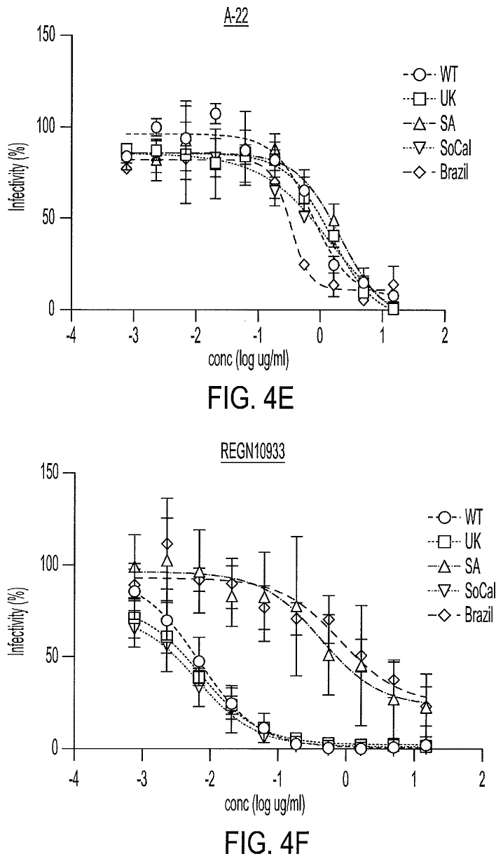

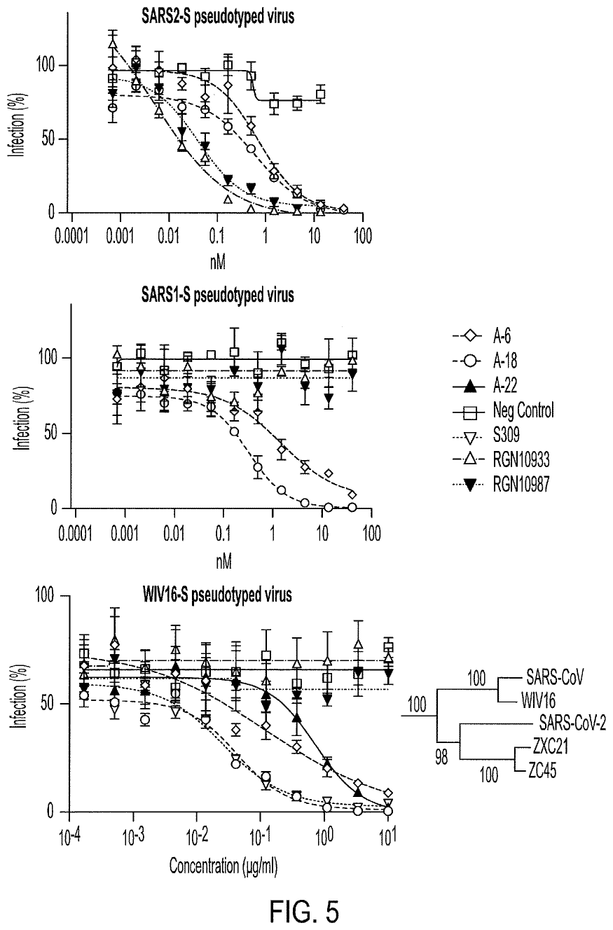

Results show that the antibodies bind to all SARS-CoV-2 RBD variants of concern and all tested variants of interest (FIGS. 4A-4F, Table 5). Additionally, results show that the selected mAbs also have comparable neutralization to coronavirus family members SARS-COV-1 and WIV16, which indicates the breadth of neutralization and that the antibodies target a conserved epitope (FIG. 5, Table 6).

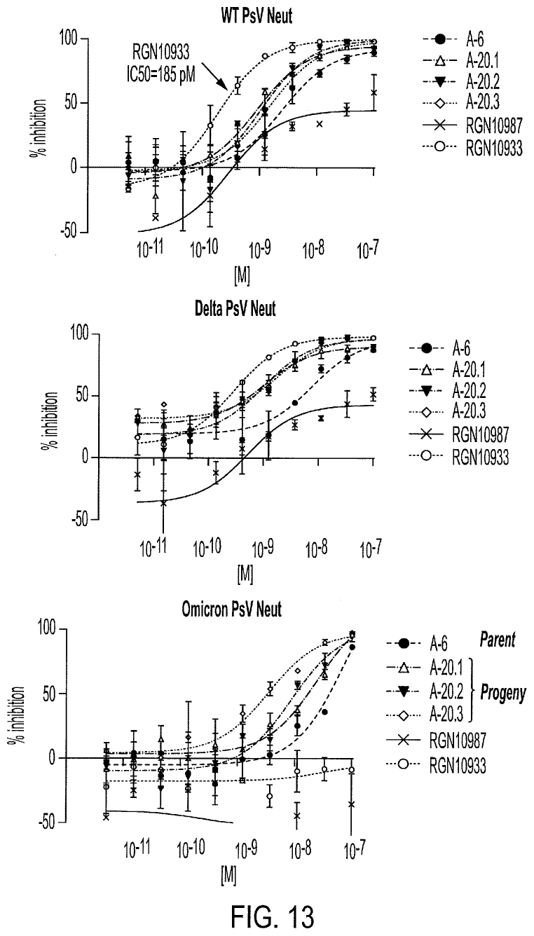

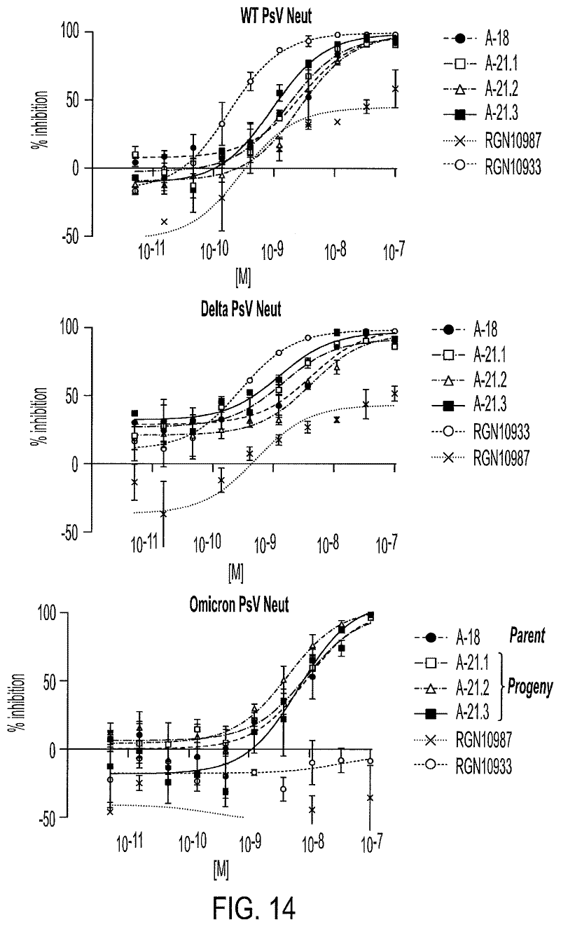

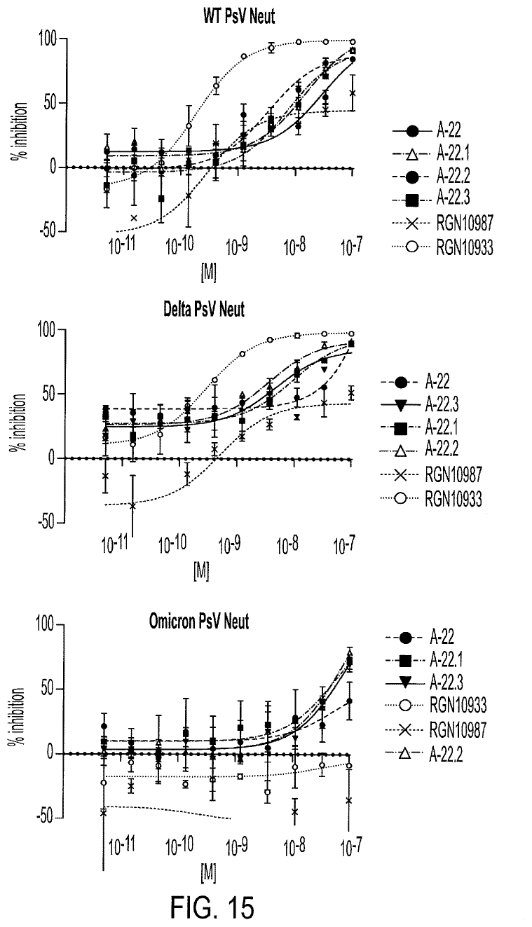

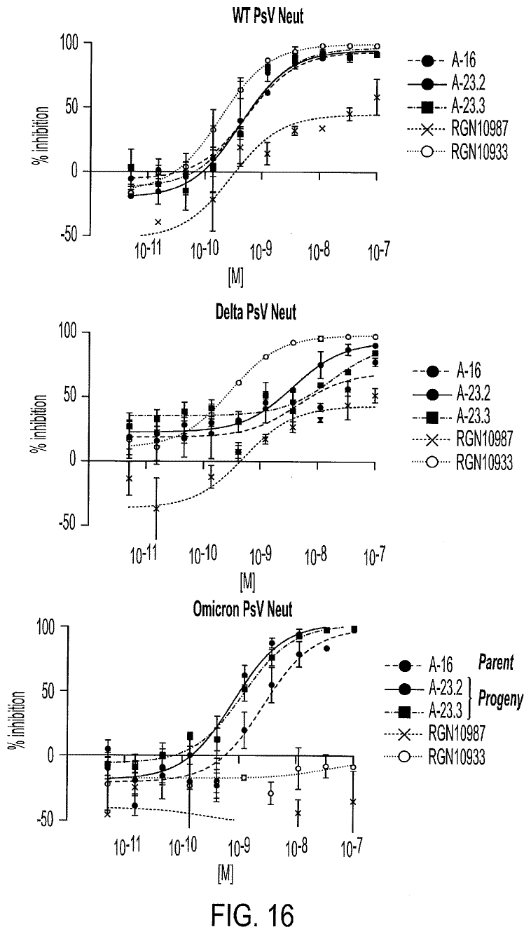

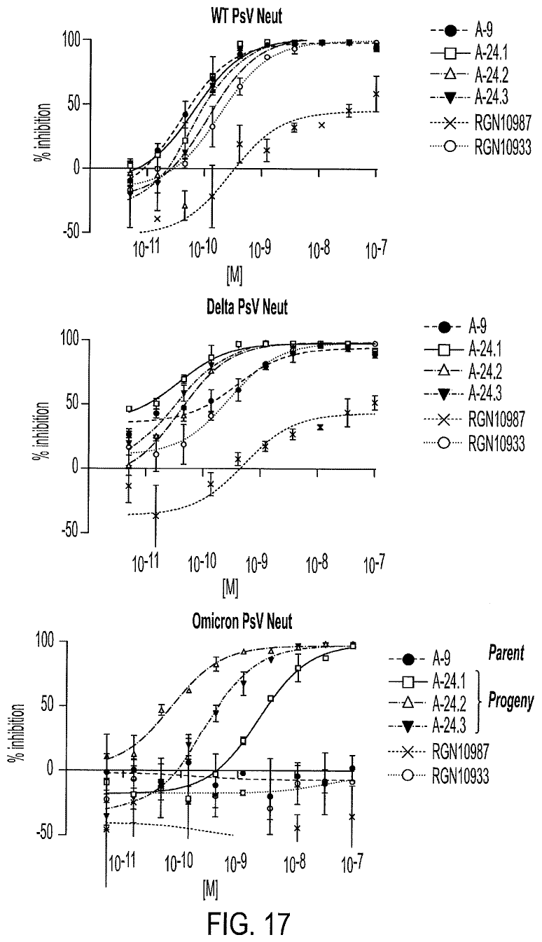

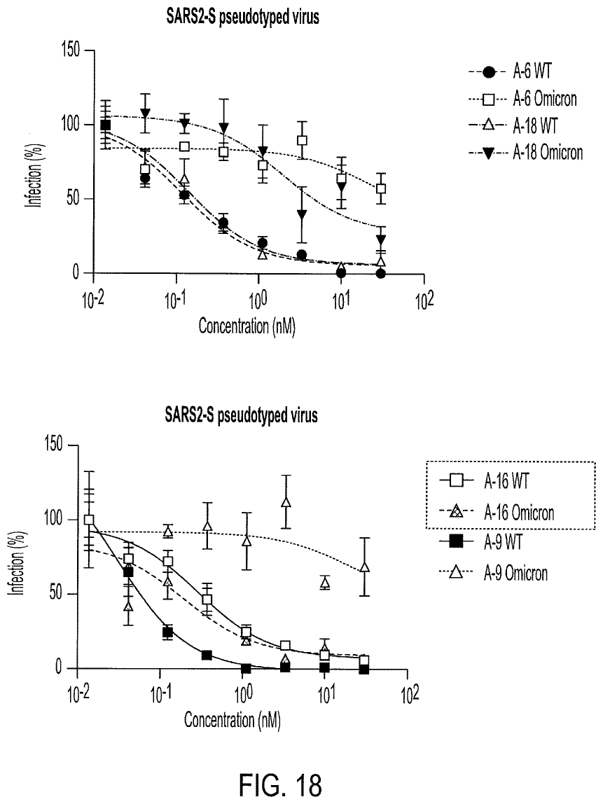

PsV assays were also performed on select clonal lineages against WT, Delta and Omicron SARS-CoV-2 variants. Clonal lineages of A-6 (FIG. 13), A-18 (FIG. 14), A-22 (FIG. 15), A-16 (FIG. 16) and A-9 (FIG. 17) were tested. Results confirmed that progeny of A-6 and A-18 showed improved PsV IC50 to WT, Delta, and Omicron variants (Table 8). Progeny of A-22 resulted in higher affinity binding, but less potent PsV neutralization. Progeny of A-16 were the most potent mAbs in Class 3 (binding outside ACE2 epitope). Finally, progeny of A-9 resulted in extremely potent Class 1 mAbs with activity to the SARS-CoV-2 Omicron variant. Parental antibodies were also independently tested to confirm Omicron activity (FIG. 18, Table 9). Additional data (not shown) demonstrate that the tested antibodies are not poly specific.

### Example 4. Live Virus Neutralization Assessment of Monoclonal Antibodies

Live viral assays were performed by making a serial dilution of the sample (containing SARS-CoV-2-neutralizing antibodies) over a total of ten 2-fold dilution steps. These 10 dilutions were mixed with an equal amount of authentic SARS-CoV-2 and incubated. The 10 dilutions were added to cells seeded in a 96-well-plate, with each dilution added to 8 wells of a distinct column (i.e., 8-fold replicates for each sample dilution). After incubation to allow for virus replication, the plate was evaluated microscopically; each well is examined to document whether a cytopathogenic effect (CPE, due to virus replication) is observed (+) or not observed (−).

From the number of (−) wells and the starting dilution, a neutralization titer was calculated, specifying the sample dilution X where 50% of the cells are protected from virus infection due to neutralization of the virus by antibodies.

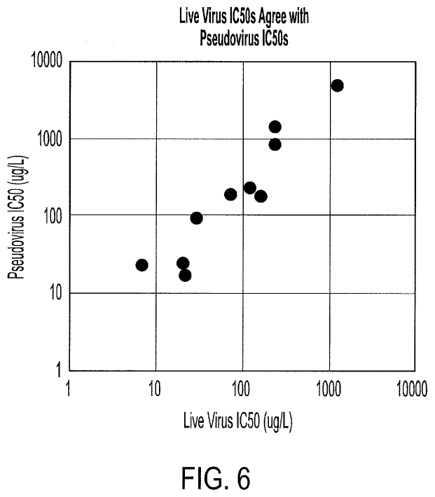

In order to validate the results obtained from PsV assays, IC50 values obtained from live virus assays were compared to IC50 values obtained from PsV assays (Table 7). As shown by the near-linear relationship in FIG. 6, pseudovirus IC50 values agreed well with live viral IC50 assay results.

### Example 5: In Vivo Virus Neutralization Assay in Mice

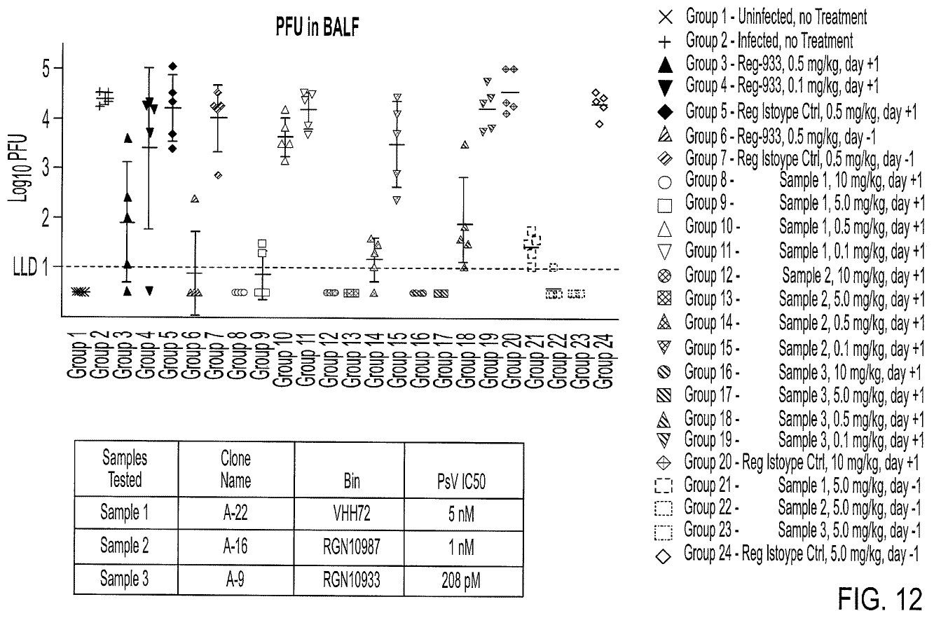

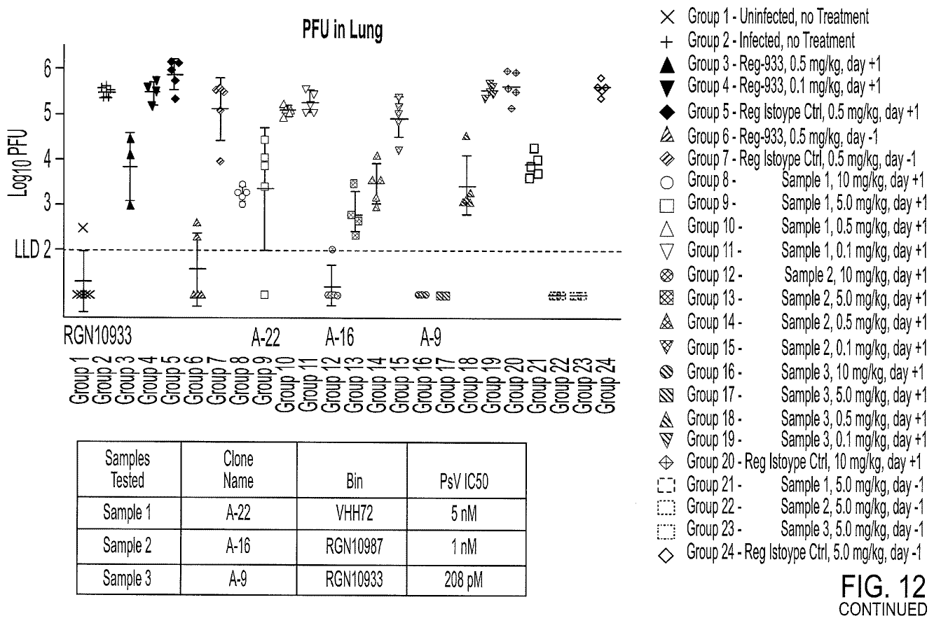

K18-hACE2 transgenic mice were immunized with protein preparations of A-9, A-16, A-22 or RGN10933 either the day prior (day −1) or day after (day 1) infection with live, SARS-CoV-2 USA-WA1/2020 (day 0). Mice were taken down for viral titer determination on day 4 after infection. Mice were dosed with 10 mg/kg, 5 mg/kg, 0.5 mg/kg or 0.1 mg/kg of the antibody of interest. Plaque-forming units (PFU) were measured in broncho-alveolar lavage fluid (BALF) and lung tissue. A-9, A-16, and A-22 all resulted in viral neutralization (FIG. 12). A-9 and A-16 produced viral neutralization equivalent to RGN10933 (Table 8).

### Example 6: Affinity Maturation of SARS-CoV-2 Neutralizing Antibodies

Antibodies that effectively neutralized SARS-CoV-2, SARS-COV-2 variants, and other coronavirus family members including SARS-COV-1 (Table 3) were further developed by generating library diversity around a single parental antibody and performing affinity selections to enrich improved progeny antibodies. Libraries were generated through oligo assembly of antibody variable regions that included degenerate codon diversity in the complementarity-determining regions (CDRs) of the heavy chain (HC) or light chain (LC). The library assembly PCR of the variable region together with digested antibody expression vectors were transformed into yeast already carrying the appropriate HC or LC pair. These libraries containing CDR diversity based around a single parental antibody were then pressured over multiple rounds of FACS to sort out the highest affinity binders to SARS-CoV-2.

Additional affinity improvements were also obtained by combining the best heavy chains from HC library selections with the best light chains from LC library selections into a mixed HC/LC library. These mixed libraries were taken through additional FACS selections to further enrich the highest affinity progeny with combined, complimentary modifications in HC and LC. Data obtained during affinity maturation is shown in Table 10. Sequences of antibodies post-affinity maturation are shown in Table 2.

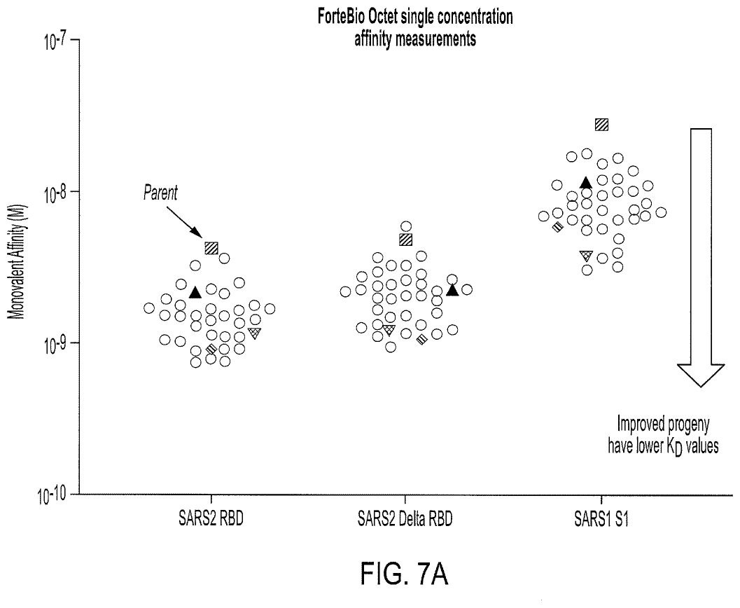

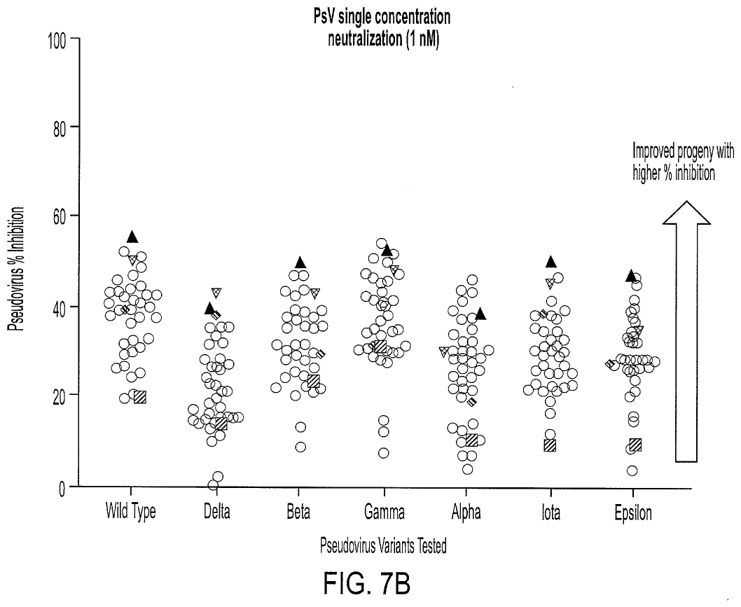

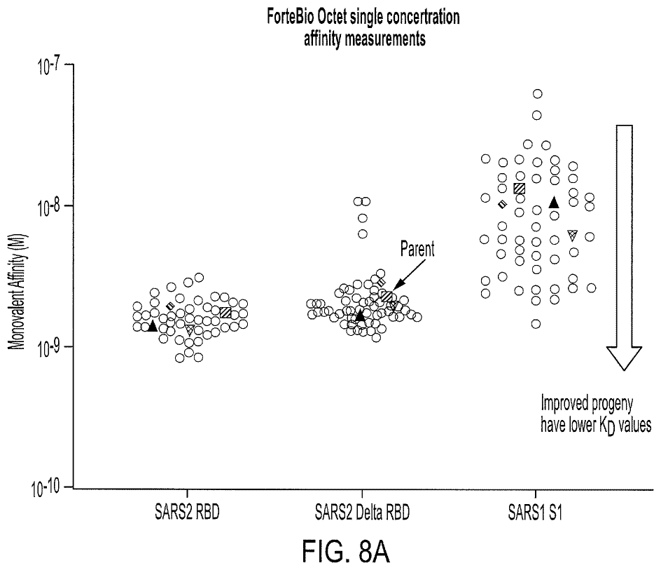

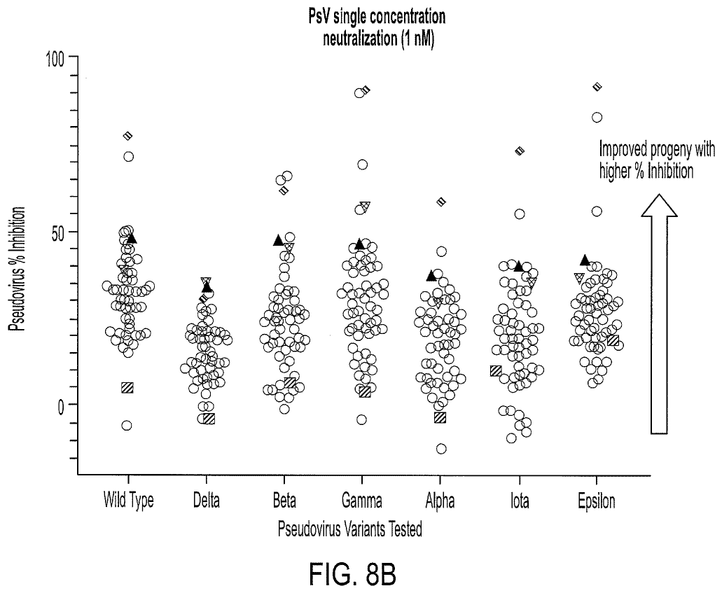

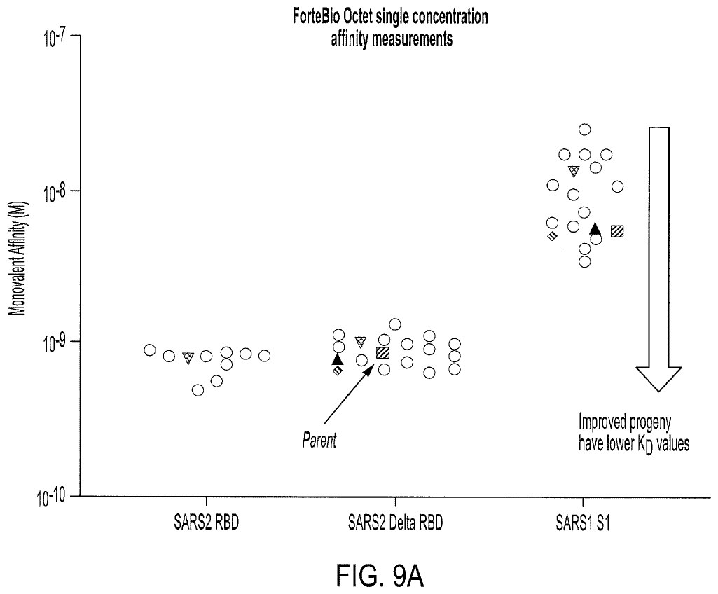

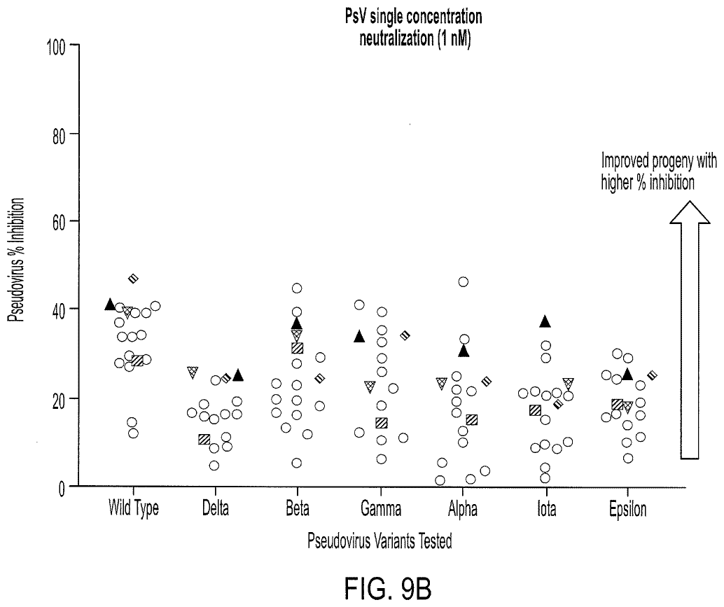

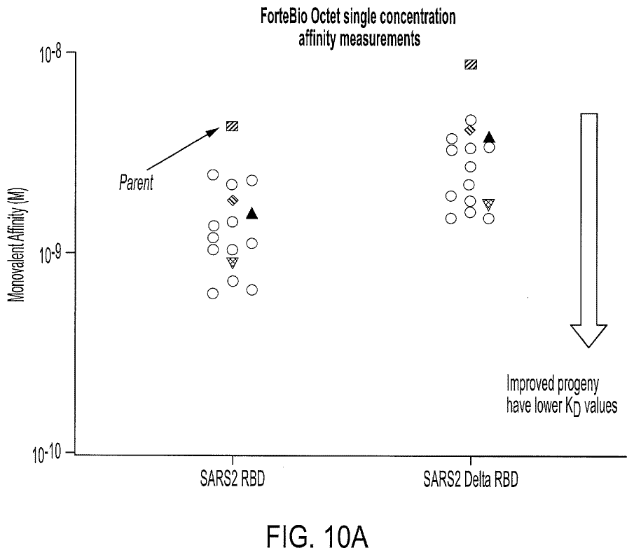

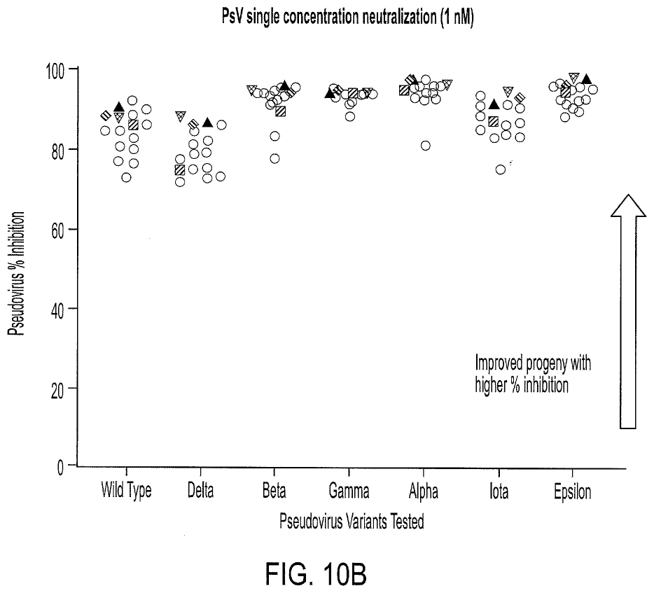

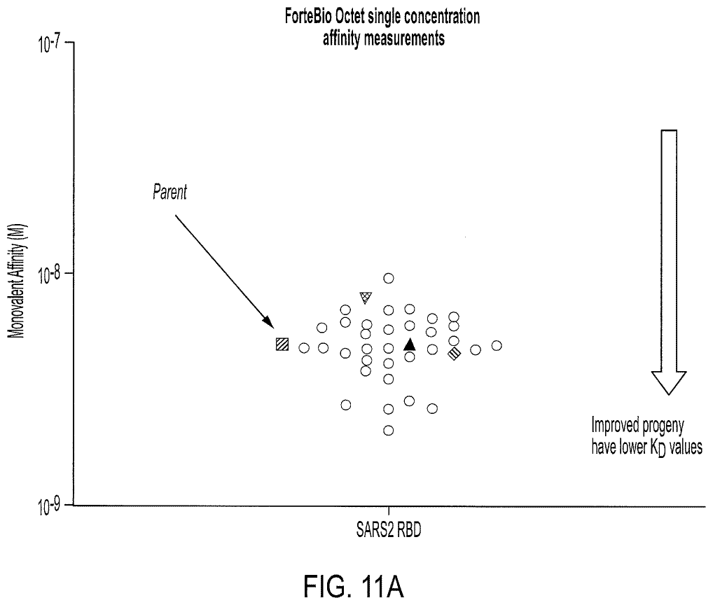

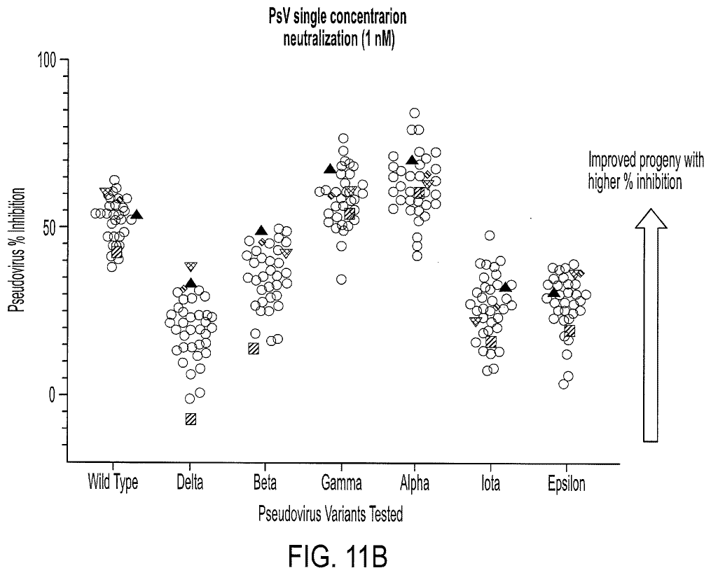

ForteBio OCTET single concentration affinity measurements and PsV single concentration neutralization assays were performed as described herein on the progeny of A-6 (FIG. 7), A-18 (FIG. 8), A-22 (FIG. 9), A-9 (FIG. 10), and A-16 (FIG. 11) after one round of affinity maturation.

Improved progeny exhibited lower K_{D} values and higher percentage pseudovirus inhibition. mAbs were tested for monovalent affinity against the SARS-COV-2 RBD, SARS-COV-2 Delta variant RBD, and/or SARS-COV-1 S1 protein. Progeny of A-6 and A-18 showed improved binding affinity to both SARS-COV-2 and SARS-COV-1 (FIGS. 7 and 8), while progeny of A-22 showed similar binding affinity to both strains (FIG. 9). Progeny of A-9 and A-16 showed improved binding affinity to SARS-COV-2, but were not tested against SARS-COV-1. mAbs were also tested for pseudovirus neutralization against the wild type, Delta, Beta, Gamma, Alpha, Iota, and Epsilon SARS-CoV-2 variants. Progeny of all antibodies showed improved affinity and PsV neutralization to all tested SARS variants (FIGS. 7A-11B). Additionally, several clones were tracked across all tests (e.g., the triangle, square, diamond and upside down triangle shown in FIGS. 7A-11B) and demonstrated improvements in binding affinity and neutralization activity against different SARS clones, relative to the respective parental clones.

After a second round of affinity maturation, affinity measurements and PSV neutralization assays to WT, Delta and Omicron SARS-CoV-2 variants were performed as described herein on the parent and progeny of A-6 (FIG. 13), A-18 (FIG. 14), A-22 (FIG. 15), A-16 (FIG. 16), and A-9 (FIG. 17). Progeny of A-9 and A-18 showed improved PsV IC50 to all variants tested (FIGS. 14-15). Progeny of A-22 were higher affinity binders and have some activity for PsV neutralization (FIG. 15). Progeny of A-16 were the most potent mAbs binding outside the ACE2 epitope (Class 3) (FIG. 16). Progeny of A-9 were highly potent Class 1 mAbs and had activity to omicron (FIG. 17). Parental antibodies were then independently tested for activity to the Omicron SARS-CoV-2 variant. All parental antibodies showed neutralization activity to Omicron (FIG. 18).

### Example 7: BLI Binding Kinetics of Purified Anti-SARS-CoV-2 Antibodies

Binding of SARS-CoV2 antibodies to various RBD mutants was determined using the OCTET HTX. All steps of the assay were performed in 40 mM HEPES, 150 mM NaCl, 0.05% Tween-20, 0.05% BSA, pH 7.4 buffer or PBS-BSA buffer. Anti-human Fe capture biosensors (Sartorius, cat #18-5060) were first primed in buffer before the start of the assay. ACE2-Fc and antibodies were loaded at 4 μg/ml and 2 μg/ml, respectively, for 300 s followed by buffer baseline for 600 s. Sensors were then placed in solution with RBD mutants at 70 nM and 35 nM, and dissociation was monitored for 300 s or up to 600 s in assay buffer. Sensorgrams were evaluated in the Data Analysis HT software for binding of the antibodies and ACE2-Fc to RBD variants. Results are shown in Table 4.

### Example 8: SPR Binding Kinetics of Purified Anti-SARS-CoV-2 Antibodies

### With Capture:

Kinetics and equilibrium constants were determined for purified SARS-CoV2-antibodies using the BIACORE 8k+ system. Binding experiments were performed in 1×HBS-EP+pH 7.4 running buffer (10 mM HEPES, 150 mM NaCl, 3 mM EDTA, 0.05% v/v Surfactant P20) at 25° C. A BIACORE CM5 sensor chip was first prepared by amine coupling anti-human Fc (Jackson, cat #109-005-098) to capture anti-SARS-CoV2 antibodies. RBD reagents were diluted to a top concentration of 50 nM in running buffer. Single concentrations of various RBD variants were then injected for 300 s at a flow rate of 30 ul/min while dissociation was monitored for 600 s by flowing over HBS-EP+ buffer. At the end of each cycle, the capture surface was regenerated by injecting 10 mM glycine-HCl pH 1.5 for 30 s at a flow rate of 30 ul/min. Binding sensorgrams were fit to a 1:1 binding model using the BIACORE Insight Evaluation Software. Association rate (ka), dissociation rate (kd), and equilibrium constant (KD) were determined using the same software.

### Direct Immobilization:

Kinetics and equilibrium constants were determined for purified SARS-CoV2-antibodies using the BIACORE 8k+ system. Binding experiments were performed in 1×HBS-EP+pH 7.4 running buffer (10 mM HEPES, 150 mM NaCl, 3 mM EDTA, 0.05% v/v Surfactant P20) at 25° C. A BIACORECM5 sensor chip was first prepared by amine coupling SARS CoV2 antibodies. RBD reagents were diluted to a top concentration of 50 nM in running buffer. Single concentrations of various RBD variants were then injected for 300 s at a flow rate of 30 μl/min while dissociation was monitored for 600 s by flowing over HBS-EP+ buffer. At the end of each cycle, the capture surface was regenerated by injecting 10 mM glycine-HCl pH 1.5 for 30 s at a flow rate of 30 μl/min. Binding sensorgrams were fit to a 1:1 binding model using the BIACORE Insight Evaluation Software. Association rate (ka), dissociation rate (kd), and equilibrium constant (KD) were determined using the same software. Obtained select antibody monovalent KD values are shown in Table 5.

### Tables

TABLE 1
Parental Antibodies

| | | | SEQ |
|---|---|---|---|
| | Antibody | | ID |
| Identifier | Part | Sequence | NO: |
| A-1 | VH CDR1 | FAFSTYWMH | 1 |
| | VH CDR2 | RINGDGSGTNYADSVKG | 2 |
| | VH CDR3 | TRTAGRGAYDFGRFDP | 3 |
| | VL CDR1 | RSSQSLLHSNGYNYLD | 4 |
| | VL CDR2 | LGSNRAS | 5 |
| | VL CDR3 | MQALQIPIT | 6 |
| | VH | EVQLVESGGGVVQPGGSLRLSCAASGFAFSTYWM | 7 |
| | | HWVRQAPGKGLVWVSRINGDGSGTNYADSVKGRF | |
| | | TISRDNAKSTLYLQMNSLRTEDTAVYYCTRTAGR | |
| | | GAYDFGRFDPWGQGTLVTVSS | |
| | VL | DIVLTQTPLSLPVTPGEPASISCRSSQSLLHSNGY | 8 |
| | | NYLDWYLQKAGQSPQLLVFLGSNRASGVPDRFSGS | |
| | | ASGTDFTLKISRVEAEDVGVYYCMQALQIPITFGQ | |
| | | GTRLEIK | |
| A-2 | VH CDR1 | FTVSSNYMS | 9 |
| | VH CDR2 | IIYSGGSTYYADSVKG | 10 |
| | VH CDR3 | ARAPSGEYGDPIYY | 11 |
| | VL CDR1 | TLSSGHSTYTIA | 12 |
| | VL CDR2 | LNSDGSHSKGD | 13 |
| | VL CDR3 | QTWGTGNHWV | 14 |
| | VH | EVQLVESGGGLIQPGGSLRLSCAASGFTVSSNYMS | 15 |
| | | WVRQAPGKGLEWVSIIYSGGSTYYADSVKGRFTIS | |
| | | RDNSKNTLYLQMNSLRAEDTAVYYCARAPSGEYG | |
| | | DPIYYWGQGTLVTVSS | |
| | VI | QSVLTQPPSASASLGASVKLTCTLSSGHSTYTIAW | 16 |
| | | HQQQPEKGPRYLMKLNSDGSHSKGDGIPDRESGSS | |
| | | SGAERYLTISSLQSEDEADYYCQTWGTGNHWVFG | |
| | | GGTKLTVL | |
| A-3 | VH CDR1 | GSINSRSYYWG | 17 |
| | VH CDR2 | SIYYSGSTYYNPSLKS | 18 |
| | VH CDR3 | ARLEQWELPGGWFDP | 19 |
| | VL CDR1 | TRSSGSIASNYVQ | 20 |
| | VL CDR2 | EDNQRPS | 21 |
| | VL CDR3 | QSSDSSNVV | 22 |
| | VH | EVQLLESGPGLVKPSETLSLTCTVSGGSINSRSYY | 23 |
| | | WGWIRQPPGKGLEWIGSIYYSGSTYYNPSLKSRVT | |
| | | ISVDTSKNQFSLKLSSVIAADTAVYFCARLEQWEL | |
| | | PGGWFDPWGQGTLVTVSS | |
| | VL | NFMLTQPHSVSESPGKTVTISCTRSSGSIASNYVQW | 24 |
| | | YQQRPGSAPTTVIYEDNQRPSGVPDRFSGSIDISSN | |
| | | SASLTISGLKTEDEADYSCQSSDSSNVVFGGGTKL | |
| | | TVL | |
| A-4 | VH CDR1 | FTVSSNYMN | 25 |
| | VH CDR2 | VIYSGGSTYYADSVKG | 26 |
| | VH CDR3 | AREVYSYFDY | 27 |
| | VL CDR1 | RASQGISSYLA | 28 |
| | VL CDR2 | AASTLQS | 29 |
| | VL CDR3 | QQLNSYPPHT | 30 |
| | VH | EVQLVESGGGLIQPGGSLTLSCAASGFTVSSNYMN | 31 |
| | | WVRQAPGKGLEWVSVIYSGGSTYYADSVKGRFTI | |
| | | SRDNSKNTLYLQMNSLRAEDTAVYYCAREVYSYF | |
| | | DYWGQGTLVTVSS | |
| | VL | DIQLTQSPSFLSASVGDRVTITCRASQGISSYLAWY | 32 |
| | | QQKPGKAPKLLIYAASTLQSGVPSRFSGSGSGTEFT | |
| | | LTISRLQPEDLATYYCQQLNSYPPHTFGQGAKLEI | |
| | | K | |
| A-5 | VH CDR1 | FIVSRNYMS | 33 |
| | VH CDR2 | VIYSGGNTYYADSVKG | 34 |
| | VH CDR3 | ARDLRGPGCFDF | 35 |
| | VL CDR1 | QASQDISNFLN | 36 |
| | VL CDR2 | DASNLET | 37 |
| | VL CDR3 | QQYDNLRVT | 38 |
| | VH | QVQLVESGGGLIQPGESLRLSCAASEFIVSRNYMS | 39 |
| | | WVRQAPGKGLEWVSVIYSGGNTYYADSVKGRFTI | |
| | | SRDNSKNTLYLQMNSLRAEDTAVYYCARDLRGPG | |
| | | CFDFWGQGTTVTVSS | |
| | VL | DIQVTQSPSSLSASVGDRVTITCQASQDISNFLNWY | 40 |
| | | QQKPGKAPKLLIYDASNLETGVPSRFSASGSGTEFT | |
| | | FTISSLQPEDIATYYCQQYDNLRVTFGGGTKLEIK | |
| A-6 | VH CDR1 | FTFSNFAMH | 41 |
| | VH CDR2 | VILYDGINKYYADSVKG | 42 |
| | VH CDR3 | ARAQNYYDRSGTLQLDAFDI | 43 |
| | VL CDR1 | RASQGIRNDLG | 44 |
| | VL CDR2 | AASSLQS | 45 |
| | VL CDR3 | LQDYNYPLT | 46 |
| | VH | QVQLVQSGGGVVQPGRSLRLSCAASGFTFSNFAM | 47 |
| | | HWVRQAPGKGLEWVAVILYDGINKYYADSVKGRF | |
| | | TISRGNSKNTLSLQMNSLRAEDTAVYYCARAQNY | |
| | | YDRSGTLQLDAFDIWGQGTMVTVSS | |
| | VL | DIVMTQSPSSLSASVGDRVTITCRASQGIRNDLGW | 48 |
| | | YQQKPGKAPKLLIYAASSLQSGVPSRFSGSGSGTD | |
| | | FTLTISSLQPEDFATYYCLQDYNYPLTFGGGTKVE | |
| | | IK | |
| A-7 | VH CDR1 | YTFTFYYIH | 49 |
| | VH CDR2 | WISPISGATNYAQTFQG | 50 |
| | VH CDR3 | AREKYYDSSGSSDY | 51 |
| | VL CDR1 | SGSSSNIGNNYVS | 52 |
| | VL CDR2 | ENNKRPS | 53 |
| | VL CDR3 | GTWESSLSAPRV | 54 |
| | VH | EVQLVESGAEVRKPGASVKVSCKASGYTFTFYYI | 55 |
| | | HWVRQAPGQGLEWMGWISPISGATNYAQTFQGR | |
| | | VTMTRDTSITTAYMELSSLTSDDTAVYYCAREKY | |
| | | YDSSGSSDYWGQGTLVTVSS | |
| | VL | QPVLTQPPSVSAAPGQNVTIFCSGSSSNIGNNYVS | 56 |
| | | WYQQLPGPAPKLLIYENNKRPSGIPDRFSGSKSGTS | |
| | | ATLGITGLQTGDEADYYCGTWESSLSAPRVFGSGT | |
| | | KLTVL | |
| A-8 | VH CDR1 | FTFSSYAMS | 57 |
| | VH CDR2 | AISGSGNSTSYADSVKG | 58 |
| | VH CDR3 | AKGLGTRSFNWGAFDY | 59 |
| | VL CDR1 | RASQGISYSLA | 60 |
| | VL CDR2 | AASNLES | 61 |
| | VL CDR3 | QQYYSTPWT | 62 |
| | VH | EVQLLESGGGLVQPGGSLRLSCAASGFTFSSYAMS | 63 |
| | | WVRQAPGKGLEWVSAISGSGNSTSYADSVKGRFT | |
| | | LSRDNSKNTLYLQMNSLRAEDTAIYYCAKGLGTR | |
| | | SFNWGAFDYWGQGTLVTVSS | |
| | VL | DIRVTQSPSSLSASVGDRVTITCRASQGISYSLAWY | 64 |
| | | QQKPGKAPKLLLYAASNLESGVPSRFSGSGSGTDY | |
| | | TLTVSSLQPEDFATYYCQQYYSTPWTFGQGTKVEI | |
| | | K | |
| A-9 | VH CDR1 | FTFADYAMS | 65 |
| | VH CDR2 | FIRSKPYGGTTEYAASVKG | 66 |
| | VH CDR3 | SRDPWYCSGGDCYAVTGSWFDP | 67 |
| | VL CDR1 | QASQDIKKYLN | 68 |
| | VL CDR2 | DVSNLET | 69 |
| | VL CDR3 | QQYDNLPQT | 70 |
| | VH | EVQLVESGGGLVKPGRSLRLSCAASGFTFADYAM | 71 |
| | | SWFRQAPGKGLEWVGFIRSKPYGGTTEYAASVKG | |
| | | RFTISRDDSKSIAYLQMNSLKTEDTAVYYCSRDPW | |
| | | YCSGGDCYAVTGSWFDPWGQGTLVTVSS | |
| | VL | DIVLTQSPSSLSASVGDRVTITCQASQDIKKYLNW | 72 |
| | | YQQKPGKAPKLLIYDVSNLETGVPSRFSGSGSGTD | |
| | | FTFTISSLQPEDVAIYYCQQYDNLPQTFGQGTKVDI | |
| | | K | |
| A-10 | VH CDR1 | FTFSSYGMH | 73 |
| | VH CDR2 | VISYDGSNKYYTDSVKG | 74 |
| | VH CDR3 | AKDLPREVYYDILTGYDNPYYYYGMDV | 75 |
| | VL CDR1 | RASQSVSSSYLA | 76 |
| | VL CDR2 | GASTRAT | 77 |
| | VL CDR3 | QQYGSSPRWT | 78 |
| | VH | QVQLVQSGGGVVQPGRSLRLSCAASGFTFSSYGM | 79 |
| | | HWVRQAPGKGLEWVAVISYDGSNKYYTDSVKGR | |
| | | FTISRDNSKNTLYLQMNSLRAEDTAVYYCAKDLP | |
| | | REVYYDILTGYDNPYYYYGMDVWGQGTTVTVSS | |
| | VL | EIVMTQSPGTLSLSPGERATLSCRASQSVSSSYLAW | 80 |
| | | YQQKPGQAPRLLIYGASTRATGIPDRFSGSGSGTDF | |
| | | TLTISRLEPEDFAVYYCQQYGSSPRWTFGQGTKVE | |
| | | IK | |
| A-11 | VH CDR1 | GTFSRYAIS | 81 |
| | VH CDR2 | GIIPVFGIVHYAQKFQG | 82 |
| | VH CDR3 | ARDRQQQPSPYYYHGMDV | 83 |
| | VL CDR1 | QASQDISNYLN | 84 |
| | VL CDR2 | DASNLET | 37 |
| | VL CDR3 | QQYDNLPL | 85 |
| | VH | EVQLVESGAEVKKPGSSVMVSCKASGGTFSRYAI | 86 |
| | | SWVRQAPGQGLEWMGGIIPVFGIVHYAQKFQGRV | |
| | | TITADKSTSTAYMELSSLRSEDTAAYYCARDRQQQ | |
| | | PSPYYYHGMDVWGQGTTVTVSS | |
| | VL | DIRLTQSPSSLSASVGDRVTITCQASQDISNYLNWY | 87 |
| | | QQKPGKAPKLLIYDASNLETGVPTRFSGSGSGTDF | |
| | | TFTISSLQPEDIATYYCQQYDNLPLFGGGTKVEIK | |
| A-12 | VH CDR1 | LTVSSNYMS | 88 |
| | VH CDR2 | LIYSGGSTYYADSVKG | 89 |
| | VH CDR3 | ARGELGIPYGMDV | 90 |
| | VL CDR1 | SGGSSNVGDNFVS | 91 |
| | VL CDR2 | DNNRRPS | 92 |
| | VL CDR3 | GTWDSSLSAGV | 93 |
| | VH | EVQLLESGGGLVQPGGSLRLSCAVSGLTVSSNYM | 94 |
| | | SWVRQAPGKGLEWVSLIYSGGSTYYADSVKGRFT | |
| | | ISRDNSKNTLYLQMNSLRAEDTAVYYCARGELGIP | |
| | | YGMDVWGQGTTVTVSS | |
| | VL | QSVLTQPPSVSAAPGQKVTISCSGGSSNVGDNFVS | 95 |
| | | WYQQLPGTAPKLLIYDNNRRPSGIPDRESGSKSGTS | |
| | | ATLGITGLQTGDEADYYCGTWDSSLSAGVFGGGT | |
| | | KVTVL | |
| A-13 | VH CDR1 | YTFTDYYMH | 96 |
| | VH CDR2 | WINPNSGGTNSAQKFQG | 97 |
| | VH CDR3 | ARDSHLAMVRGDFEY | 98 |
| | VL CDR1 | TGTSSDVESYNVVS | 99 |
| | VL CDR2 | EVSKRPS | 100 |
| | VL CDR3 | CSYAGNSTWV | 101 |
| | VH | QVQLVQSGAEVKKPGASVKVSCKASGYTFTDYY | 102 |
| | | MHWVRQAPGQGLEWMGWINPNSGGTNSAQKFQ | |
| | | GRVTMTRDTSINTAYMELSRLRSDDTAVYFCARD | |
| | | SHLAMVRGDFEYWGQGTLVTVSS | |
| | VL | QSVLTQPASVSGSPGQSITISCTGTSSDVESYNVVS | 103 |
| | | WYQQHPGKAPKLMIYEVSKRPSGVSHRFSGSKSG | |
| | | NTASLTISGLQAEDESDYYCCSYAGNSTWVFGGG | |
| | | TKLTVL | |
| A-14 | VH CDR1 | FTFSTYGMH | 104 |
| | VH CDR2 | VIWYDGSNKYYADSVKG | 105 |
| | VH CDR3 | ARSVDGDYLEYFQH | 106 |
| | VL CDR1 | TGTSSDVGGYNYVS | 107 |
| | VL CDR2 | DVSKRPS | 108 |
| | VL CDR3 | CSYAGGYTNYV | 109 |
| | VH | EVQLVESGGGVVQPGRSLRLSCAASGFTFSTYGM | 110 |
| | | HWVRQAPGKGLEWVAVIWYDGSNKYYADSVKG | |
| | | RFTISRDNSKNTLYLQMNSLRAEDTAVYYCARSV | |
| | | DGDYLEYFQHWGQGTLVTVSS | |
| | VL | QPVLTQPRSVSGSPGQSVTISCTGTSSDVGGYNYV | 111 |
| | | SWYQQHPGKAPKLMIYDVSKRPSGVPDRFSGSKS | |
| | | GNTASLTISGLQAEDEADYYCCSYAGGYTNYVFG | |
| | | TGTKLTVL | |
| A-15 | VH CDR1 | YTSSSYYMH | 112 |
| | VH CDR2 | IINPGGTSTSYAQKFQG | 113 |
| | VH CDR3 | AIGYCSTTSCRWGDAFDI | 114 |
| | VL CDR1 | RASQSISSYLN | 115 |
| | VL CDR2 | AASSLQS | 45 |
| | VL CDR3 | QQSSTTPPT | 116 |
| | VH | QVQLVQSGAEVKKPGASVKVSCKASGYTSSSYYM | 117 |
| | | HWVRQAPGQGPEWMGIINPGGTSTSYAQKFQGR | |
| | | VTMTRDTSTSTVYMELSSLRSEDTAVYYCAIGYCS | |
| | | TTSCRWGDAFDIWGQGTMVTVSS | |
| | VL | DIQVTQSPSSLSASVGDRVTITCRASQSISSYLNWY | 118 |
| | | QQKPEKAPNLLIYAASSLQSGVPSRFSGSGSGTDFT | |
| | | LTISSLQPEDFATYYCQQSSTTPPTFGQGTKVEIK | |
| A-16 | VH CDR1 | FTFDDYAMH | 119 |
| | VH CDR2 | GISWNSGTIGYADSVKG | 120 |
| | VH CDR3 | GKDRKREDPSLGGMDV | 121 |
| | VL CDR1 | GGNNIGSKSVH | 122 |
| | VL CDR2 | DDTDRPS | 123 |
| | VL CDR3 | QVWDSSSDHYV | 124 |
| | VH | QVQLVESGGGLVQPGRSLRLSCAASGFTFDDYAM | 125 |
| | | HWVRQAPGKGLEWVSGISWNSGTIGYADSVKGRF | |
| | | IISRDNAKNSLYLQMNSLRGEDMALYYCGKDRKR | |
| | | EDPSLGGMDVWGQGTTVTVSS | |
| | VL | SYELTQPPSVSVAPGQTARITCGGNNIGSKSVHWY | 126 |
| | | QQKPGQAPVLVVYDDTDRPSGIPERFSGSNSGNTA | |
| | | TLTISRVEAGDEADYYCQVWDSSSDHYVFGTGTK | |
| | | LTVL | |
| A-17 | VH CDR1 | GSISSYYWS | 127 |
| | VH CDR2 | YIYYSGSTNYNPSLKS | 128 |
| | VH CDR3 | AKSASDEPPPSTAAAGSHYYYYGMDV | 129 |
| | VL CDR1 | RSSQSLLHSNGYNYLD | 4 |
| | VL CDR2 | LGSNRAS | 5 |
| | VL CDR3 | MQALQTPNT | 130 |
| | VH | EVQLQESGPGLVKPSETLSLTCTVSGGSISSYYWS | 131 |
| | | WIRQPPGKGLEWIGYIYYSGSTNYNPSLKSRVTISV | |
| | | GTSKNQFSLKLSSVTAADTAVYYCAKSASDEPPPS | |
| | | TAAAGSHYYYYGMDVWGQGTTVTVSS | |
| | VL | EIVMTQSPLSLPVTPGEPASISCRSSQSLLHSNGYN | 132 |
| | | YLDWYLQKPGQSPHVLIYLGSNRASGVPDRESGS | |
| | | GSGTDFTLKISRVEAEDVGVYYCMQALQTPNTFG | |
| | | QGTKLEIK | |
| A-18 | VH CDR1 | FTFSRYWMH | 133 |
| | VH CDR2 | RINSDGSSTTYADSVKG | 134 |
| | VH CDR3 | ARELYYYDSRGAEEGEGWFDP | 135 |
| | VL CDR1 | TGISSNIGAGYDVH | 136 |
| | VL CDR2 | GNSNRPS | 137 |
| | VL CDR3 | QSSDRSLSGSV | 138 |
| | VH | EVQLLESGGGLVQPGGSLRLSCAASGFTFSRYWM | 139 |
| | | HWVRQAPGKGLVWVSRINSDGSSTTYADSVKGRF | |
| | | TISRDNAKNTLYLQMNSLRAEDTAVYYCARELYY | |
| | | YDSRGAEEGEGWFDPWGQGTLVTVSS | |
| | VL | QSVLTQPPSVSGAPGQRVTISCTGISSNIGAGYDVH | 140 |
| | | WYQQLPGTAPKLLIYGNSNRPSGVPDRFSASKSGT | |
| | | SAPLAITGLQAEDEADYYCQSSDRSLSGSVFGTGT | |
| | | KLTVL | |
| A-19 | VH CDR1 | YSFTSYWIA | 141 |
| | VH CDR2 | IIYPGDSDTSYSPSFQG | 142 |
| | VH CDR3 | ARGPNKYNWFDT | 143 |
| | VL CDR1 | SGSSSNIGSNTVN | 144 |
| | VL CDR2 | SNNQRPS | 145 |
| | VL CDR3 | AAWDDSLNGVV | 146 |
| | VH | QVQLVQSGAEVKKPGESLKISCKGSGYSFTSYWIA | 147 |
| | | WVRQMPGKGLEWMGIIYPGDSDTSYSPSFQGQVTI | |
| | | SADKSISTAYLQWSSLKASDTAMYYCARGPNKYN | |
| | | WFDTWGQGTLVTVSS | |
| | VL | SYVLTQPPSASGTPGQRVTISCSGSSSNIGSNTVNW | 148 |
| | | YQQLPGTAPKLLIYSNNQRPSGVPDRFSGSKSGTS | |
| | | ASLAISGLQSEDETDYYCAAWDDSLNGVVFGGGT | |
| | | KLTVL | |

TABLE 2
Affinity Matured Antibodies

| | | | SEQ |
|---|---|---|---|
| | Antibody | | ID |
| Identifier | Part | Sequence | NO: |
| A-20 | VH CDR1 | FTFSNFAMH | 41 |
| | VH CDR2 | VILYDGINKYYADSVKG | 42 |
| | VH CDR3 | ARAQNYYDRSGTLQLDAFDI | 43 |
| | VL CDR1 | RASQGIRNDLG | 44 |
| | VL CDR2 | AASSLQS | 45 |
| | VL CDR3 | LQDYNYPLT | 46 |
| | VH | QVQLVESGGGVVQPGRSLRLSCAASGFTFSNFAM | 149 |
| | | HWVRQAPGKGLEWVAVILYDGINKYYADSVKGR | |
| | | FTISRGNSKNTLSLQMNSLRAEDTAVYYCARAQN | |
| | | YYDRSGTLQLDAFDIWGQGTLVTVSS | |
| | VL | AIQMTQSPSSLSASVGDRVTITCRASQGIRNDLGW | 150 |
| | | YQQKPGKAPKLLIYAASSLQSGVPSRFSGSGSGTD | |
| | | FTLTISSLQPEDFATYYCLQDYNYPLTFGGGTKVEI | |
| | | K | |
| A-20.1 | VH CDR1 | FTFSNFAMH | 41 |
| | VH CDR2 | VILYDGINKYYADSVKG | 42 |
| | VH CDR3 | ARAQNYYDRSGTLQLDAFDI | 43 |
| | VL CDR1 | VASQGIRNDLG | 151 |
| | VL CDR2 | EASSLES | 152 |
| | VL CDR3 | LQDYNYPLT | 46 |
| | VH | QVQLVESGGGVVQPGRSLRLSCAASGFTFSNFAM | 149 |
| | | HWVRQAPGKGLEWVAVILYDGINKYYADSVKGR | |
| | | FTISRGNSKNTLSLQMNSLRAEDTAVYYCARAQN | |
| | | YYDRSGTLQLDAFDIWGQGTLVTVSS | |
| | VL | AIQMTQSPSSLSASVGDRVTITCVASQGIRNDLGW | 153 |
| | | YQQKPGKAPKLLIYEASSLESGVPSRFSGSGSGTDF | |
| | | TLTISSLQPEDFATYYCLQDYNYPLTFGGGTKVEIK | |
| A-20.2 | VH CDR1 | FTFDNFAMH | 154 |
| | VH CDR2 | VIEYDGINKYYADSVKG | 155 |
| | VH CDR3 | ARAQNYYDRYGTLQLDAFDI | 156 |
| | VL CDR1 | RASQGIRNDLG | 44 |
| | VL CDR2 | DASSLQS | 157 |
| | VL CDR3 | LQDYNYPLT | 46 |
| | VH | QVQLVQSGGGVVQPGRSLRLSCAASGFTFDNFAM | 158 |
| | | HWVRQAPGKGLEWVAVIEYDGINKYYADSVKGR | |
| | | FTISRGNSKNTLSLQMNSLRAEDTAVYYCARAQN | |
| | | YYDRYGTLQLDAFDIWGQGTLVTVSS | |
| | VL | AIQMTQSPSSLSASVGDRVTITCRASQGIRNDLGW | 159 |
| | | YQQKPGKAPKLLIYDASSLQSGVPSRFSGSGSGTD | |
| | | FTLTISSLQPEDFATYYCLQDYNYPLTFGGGTKVEI | |
| | | K | |
| A-20.3 | VH CDR1 | FTFDNFAMH | 154 |
| | VH CDR2 | VILYDGINDYYADSVKG | 160 |
| | VH CDR3 | ARAQNYYDRHGTLQLDAFDI | 161 |
| | VL CDR1 | RASDGIRNDLG | 162 |
| | VL CDR2 | EASSLQS | 163 |
| | VL CDR3 | LQNYNYPLT | 164 |
| | VH | QVQLVQSGGGVVQPGRSLRLSCAASGFTFDNFAM | 165 |
| | | HWVRQAPGKGLEWVAVILYDGINDYYADSVKGR | |
| | | FTISRGNSKNTLSLQMNSLRAEDTAVYYCARAQN | |
| | | YYDRHGTLQLDAFDIWGQGTLVTVSS | |
| | VL | AIQMTQSPSSLSASVGDRVTITCRASDGIRNDLGW | 166 |
| | | YQQKPGKAPKLLIYEASSLQSGVPSRFSGSGSGTDF | |
| | | TLTISSLQPEDFATYYCLQNYNYPLTFGGGTKVEIK | |
| A-21 | VH CDR1 | FTFSRYWMH | 133 |
| | VH CDR2 | RINSDGSSTTYADSVKG | 134 |
| | VH CDR3 | ARELYYYDSRGAEEGEGWFDP | 135 |
| | VL CDR1 | TGISSNIGAGYDVH | 136 |
| | VL CDR2 | GNSNRPS | 137 |
| | VL CDR3 | QSSDRSLSGSV | 138 |
| | VH | EVQLVESGGGLVQPGGSLRLSCAASGFTFSRYWM | 167 |
| | | HWVRQAPGKGLVWVSRINSDGSSTTYADSVKGRF | |
| | | TISRDNAKNTLYLQMNSLRAEDTAVYYCARELYY | |
| | | YDSRGAEEGEGWFDPWGQGTLVTVSS | |
| | VL | QSVLTQPPSVSGAPGQRVTISCTGISSNIGAGYDVH | 168 |
| | | WYQQLPGTAPKLLIYGNSNRPSGVPDRESASKSGT | |
| | | SAPLAITGLQAEDEADYYCQSSDRSLSGSVFGTGT | |
| | | KVTVL | |
| A-21.1 | VH CDR1 | FTFRRYWMH | 169 |
| | VH CDR2 | RINSDGSSTTYADSVKG | 134 |
| | VH CDR3 | ARELYYYDSRGFEEGEGWFDP | 170 |
| | VL CDR1 | TGISSNIGAGYDVH | 136 |
| | VL CDR2 | GNSNRPS | 137 |
| | VL CDR3 | QSSDRSLSGSV | 138 |
| | VH | EVQLVESGGGLVQPGGSLRLSCAASGFTFRRYWM | 171 |
| | | HWVRQAPGKGLVWVSRINSDGSSTTYADSVKGRF | |
| | | TISRDNAKNTLYLQMNSLRAEDTAVYYCARELYY | |
| | | YDSRGFEEGEGWFDPWGQGTLVTVSS | |
| | VL | QSVLTQPPSVSGAPGQRVTISCTGISSNIGAGYDVH | 168 |
| | | WYQQLPGTAPKLLIYGNSNRPSGVPDRESASKSGT | |
| | | SAPLAITGLQAEDEADYYCQSSDRSLSGSVFGTGT | |
| | | KVTVL | |
| A-21.2 | VH CDR1 | FTFSRYWMH | 133 |
| | VH CDR2 | RINSDGSSTTYADSVKG | 134 |
| | VH CDR3 | ARELYYYDSRGAEEGEGWFDP | 135 |
| | VL CDR1 | TGISSNIGAQYDVH | 172 |
| | VL CDR2 | GNSNRPR | 173 |
| | VL CDR3 | QSSDRSVSGSV | 174 |
| | VH | EVQLVESGGGLVQPGGSLRLSCAASGFTFSRYWM | 167 |
| | | HWVRQAPGKGLVWVSRINSDGSSTTYADSVKGRF | |
| | | TISRDNAKNTLYLQMNSLRAEDTAVYYCARELYY | |
| | | YDSRGAEEGEGWFDPWGQGTLVTVSS | |
| | VL | QSVLTQPPSVSGAPGQRVTISCTGISSNIGAQYDVH | 175 |
| | | WYQQLPGTAPKLLIYGNSNRPRGVPDRFSASKSGT | |
| | | SAPLAITGLQAEDEADYYCQSSDRSVSGSVFGTGT | |
| | | KVTVL | |
| A-21.3 | VH CDR1 | FTFRRYWMH | 169 |
| | VH CDR2 | RINSDGSSTTYADSVEG | 176 |
| | VH CDR3 | ARELYYYDRRGAEEGEGWFDP | 177 |
| | VL CDR1 | TGISSNIGAEYDVH | 178 |
| | VL CDR2 | GNSNRPR | 173 |
| | VL CDR3 | QVSDRYLSGSV | 179 |
| | VH | EVQLVESGGGLVQPGGSLRLSCAASGFTFRRYWM | 180 |
| | | HWVRQAPGKGLEWVSRINSDGSSTTYADSVEGRF | |
| | | TISRDNAKNTLYLQMNSLRAEDTAVYYCARELYY | |
| | | YDRRGAEEGEGWFDPWGQGTLVTVSS | |
| | VL | QSVLTQPPSVSGAPGQRVTISCTGISSNIGAEYDVH | 181 |
| | | WYQQLPGTAPKLLIYGNSNRPRGVPDRESASKSGT | |
| | | SAPLAITGLQAEDEADYYCQVSDRYLSGSVFGTGT | |
| | | KVTVL | |
| A-22 | VH CDR1 | FTFSSYDMS | 182 |
| | VH CDR2 | GISGSGAITYYTDSVKG | 183 |
| | VH CDR3 | TKENNRNTFFDY | 184 |
| | VL CDR1 | RASQTVRSDLA | 185 |
| | VL CDR2 | GASTRAT | 77 |
| | VL CDR3 | QQYYEWPPHSDS | 186 |
| | VH | EVQLVESGGGVVRPGESLRLSCAASGFTFSSYDMS | 187 |
| | | WVRQAPGEGLEWVSGISGSGAITYYTDSVKGRFTI | |
| | | SRDNSKNTLYLQMNSLRAEDTAVYYCTKENNRNT | |
| | | FFDYWGQGTLVTVSS | |
| | VL | EIVLTQSPGTLSLSPGERATLSCRASQTVRSDLAWY | 188 |
| | | QQKPGQAPRLLIYGASTRATDISDRESGSGSGTEFT | |
| | | LTISSLQSEDFAVYYCQQYYEWPPHSDSFGGGTKV | |
| | | EIK | |
| A-22.1 | VH CDR1 | FTFSSTDMS | 189 |
| | VH CDR2 | GISGSGAITYYTDSVKG | 183 |
| | VH CDR3 | TKENNRATFFDY | 190 |
| | VL CDR1 | RASQTVRSDLA | 185 |
| | VL CDR2 | GASTRAT | 77 |
| | VL CDR3 | QQYYEWPPHSDS | 186 |
| | VH | EVQLVESGGGVVRPGESLRLSCAASGFTFSSTDMS | 191 |
| | | WVRQAPGEGLEWVSGISGSGAITYYTDSVKGRFTI | |
| | | SRDNSKNTLYLQMNSLRAEDTAVYYCTKENNRAT | |
| | | FFDYWGQGTLVTVSS | |
| | VL | EIVLTQSPGTLSLSPGERATLSCRASQTVRSDLAWY | 188 |
| | | QQKPGQAPRLLIYGASTRATDISDRESGSGSGTEFT | |
| | | LTISSLQSEDFAVYYCQQYYEWPPHSDSFGGGTKV | |
| | | EIK | |
| A-22.2 | VH CDR1 | PTFSSYDMS | 192 |
| | VH CDR2 | GISGSGAITYYTDSVKG | 183 |
| | VH CDR3 | TKENNRGTFFDY | 193 |
| | VL CDR1 | SASQTVRSDLA | 194 |
| | VL CDR2 | GGSTRAT | 195 |
| | VL CDR3 | QQYYEWPPHSDS | 186 |
| | VH | EVQLVESGGGVVRPGESLRLSCAASGPTFSSYDMS | 196 |
| | | WVRQAPGEGLEWVSGISGSGAITYYTDSVKGRFTI | |
| | | SRDNSKNTLYLQMNSLRAEDTAVYYCTKENNRGT | |
| | | FFDYWGQGTLVTVSS | |
| | VL | EIVLTQSPGTLSLSPGERATLSCSASQTVRSDLAWY | 197 |
| | | QQKPGQAPRLLIYGGSTRATDISDRFSGSGSGTEFT | |
| | | LTISSLQSEDFAVYYCQQYYEWPPHSDSFGGGTKV | |
| | | EIK | |
| A-22.3 | VH CDR1 | FTFSSYDMS | 182 |
| | VH CDR2 | GISGEGAITYYTDSVKG | 198 |
| | VH CDR3 | TKENNRATFFDY | 190 |
| | VL CDR1 | RASQTVRSDLA | 185 |
| | VL CDR2 | GGSTRAT | 195 |
| | VL CDR3 | QQYYEWPPHSDS | 186 |
| | VH | EVQLVESGGGVVRPGESLRLSCAASGFTFSSYDMS | 199 |
| | | WVRQAPGEGLEWVSGISGEGAITYYTDSVKGRFTI | |
| | | SRDNSKNTLYLQMNSLRAEDTAVYYCTKENNRAT | |
| | | FFDYWGQGTLVTVSS | |
| | VL | EIVLTQSPGTLSLSPGERATLSCRASQTVRSDLAWY | 200 |
| | | QQKPGQAPRLLIYGGSTRATDISDRESGSGSGTEFT | |
| | | LTISSLQSEDFAVYYCQQYYEWPPHSDSFGGGTKV | |
| | | EIK | |
| A-23 | VH CDR1 | FTFDDYAMH | 119 |
| | VH CDR2 | GISWNSGTIGYADSVKG | 120 |
| | VH CDR3 | GKDRKREDPSLGGMDV | 121 |
| | VL CDR1 | GGNNIGSKSVH | 122 |
| | VL CDR2 | DDTDRPS | 123 |
| | VL CDR3 | QVWDSSSDHYV | 124 |
| | VH | EVQLVESGGGLVQPGRSLRLSCAASGFTEDDYAM | 201 |
| | | HWVRQAPGKGLEWVSGISWNSGTIGYADSVKGRF | |
| | | IISRDNAKNSLYLQMNSLRGEDMALYYCGKDRKR | |
| | | EDPSLGGMDVWGQGTTVTVSS | |
| | VL | SYVLTQPPSVSVAPGQTARITCGGNNIGSKSVHWY | 202 |
| | | QQKPGQAPVLVVYDDTDRPSGIPERFSGSNSGNTA | |
| | | TLTISRVEAGDEADYYCQVWDSSSDHYVFGTGTK | |
| | | VTVL | |
| A-23.1 | VH CDR1 | FTIDDYAMH | 203 |
| | VH CDR2 | GISWNGGTIGYADSVKG | 204 |
| | VH CDR3 | GKDRKREDPSLGGMDV | 121 |
| | VL CDR1 | LGNNIGSKSVH | 205 |
| | VL CDR2 | DDTDRPS | 123 |
| | VL CDR3 | QVWDSESDHYV | 206 |
| | VH | EVQLVESGGGLVQPGRSLRLSCAASGFTIDDYAM | 207 |
| | | HWVRQAPGKGLEWVSGISWNGGTIGYADSVKGR | |
| | | FIISRDNAKNSLYLQMNSLRGEDMALYYCGKDRK | |
| | | REDPSLGGMDVWGQGTTVTVSS | |
| | VL | SYVLTQPPSVSVAPGQTARITCLGNNIGSKSVHWY | 208 |
| | | QQKPGQAPVLVVYDDTDRPSGIPERFSGSNSGNTA | |
| | | TLTISRVEAGDEADYYCQVWDSESDHYVFGTGTK | |
| | | VTVL | |
| A-23.2 | VH CDR1 | FTFDDYAMH | 119 |
| | VH CDR2 | GTSWNSGTIGYADSVKG | 209 |
| | VH CDR3 | VKDRKREDPSLGGMDV | 210 |
| | VL CDR1 | GGNNIGSKSVH (SEQ ID NO:269) | 122 |
| | VL CDR2 | DDTDRPS | 123 |
| | VL CDR3 | QVWDVSSDSYV | 211 |
| | VH | EVQLVESGGGLVQPGRSLRLSCAASGFTFDDYAM | 212 |
| | | HWVRQAPGKGLEWVSGTSWNSGTIGYADSVKGR | |
| | | FIISRDNAKNSLYLQMNSLRGEDMALYYCVKDRK | |
| | | REDPSLGGMDVWGQGTTVTVSS | |
| | VL | SYVLTQPPSVSVAPGQTARITCGGNNIGSKSVHWY | 213 |
| | | QQKPGQAPVLVVYDDTDRPSGIPERFSGSNSGNTA | |
| | | TLTISRVEAGDEADYYCQVWDVSSDSYVFGTGTK | |
| | | VTVL | |
| A-23.3 | VH CDR1 | FTFDDYAMH | 119 |
| | VH CDR2 | GTSWNSGTIGYADSVKG | 209 |
| | VH CDR3 | VKDRKREDPSLGGMDV | 210 |
| | VL CDR1 | GGNNIGSKSVH | 122 |
| | VL CDR2 | DDTDRPS | 123 |
| | VL CDR3 | QVWDSSSDDYV | 214 |
| | VH | EVQLVESGGGLVQPGRSLRLSCAASGFTFDDYAM | 212 |
| | | HWVRQAPGKGLEWVSGTSWNSGTIGYADSVKGR | |
| | | FIISRDNAKNSLYLQMNSLRGEDMALYYCVKDRK | |
| | | REDPSLGGMDVWGQGTTVTVSS | |
| | VL | SYVLTQPPSVSVAPGQTARITCGGNNIGSKSVHWY | 215 |
| | | QQKPGQAPVLVVYDDTDRPSGIPERFSGSNSGNTA | |
| | | TLTISRVEAGDEADYYCQVWDSSSDDYVFGTGTK | |
| | | VTVL | |
| A-24 | VH CDR1 | FTFADYAMS | 65 |
| | VH CDR2 | FIRSKPYGGTTEYAASVKG | 66 |
| | VH CDR3 | SRDPWYCSGGDCYAVTGSWFDP | 67 |
| | VL CDR1 | QASQDIKKYLN | 68 |
| | VL CDR2 | DVSNLET | 69 |
| | VL CDR3 | QQYDNLPQT | 70 |
| | VH | EVQLVESGGGLVKPGRSLRLSCAASGFTFADYAM | 71 |
| | | SWFRQAPGKGLEWVGFIRSKPYGGTTEYAASVKG | |
| | | RFTISRDDSKSIAYLQMNSLKTEDTAVYYCSRDPW | |
| | | YCSGGDCYAVTGSWFDPWGQGTLVTVSS | |
| | VL | DIQMTQSPSSLSASVGDRVTITCQASQDIKKYLNW | 216 |
| | | YQQKPGKAPKLLIYDVSNLETGVPSRFSGSGSGTD | |
| | | FTFTISSLQPEDVAIYYCQQYDNLPQTFGQGTKVEI | |
| | | K | |
| A-24.1 | VH CDR1 | FTFADYTMS | 217 |
| | VH CDR2 | FIRSEPYGGTTEYAASVKG | 218 |
| | VH CDR3 | SRDPWYCSGGDCYAVTGSWFDP | 67 |
| | VL CDR1 | QASQDIKKYLN | 68 |
| | VL CDR2 | DVSNLET | 69 |
| | VL CDR3 | QQYDNLPQT | 70 |
| | VH | EVQLVESGGGLVKPGRSLRLSCAASGFTFADYTMS | 219 |
| | | WFRQAPGKGLEWVGFIRSEPYGGTTEYAASVKGR | |
| | | FTISRDDSKSIAYLQMNSLKTEDTAVYYCSRDPWY | |
| | | CSGGDCYAVTGSWFDPWGQGTLVTVSS | |
| | VL | DIQMTQSPSSLSASVGDRVTITCQASQDIKKYLNW | 216 |
| | | YQQKPGKAPKLLIYDVSNLETGVPSRFSGSGSGTD | |
| | | FTFTISSLQPEDVAIYYCQQYDNLPQTFGQGTKVEI | |
| | | K | |
| A-24.2 | VH CDR1 | FTFADYTMS | 217 |
| | VH CDR2 | FIRSEPYGGTTEYAASVKG | 218 |
| | VH CDR3 | SRDPWYCSGGDCYAVTGSWFDP | 67 |
| | VL CDR1 | QASQDIKQYLN | 220 |
| | VL CDR2 | DQSNLET | 221 |
| | VL CDR3 | QQYWNLPQT | 222 |
| | VH | EVQLVESGGGLVKPGRSLRLSCAASGFTFADYTMS | 219 |
| | | WFRQAPGKGLEWVGFIRSEPYGGTTEYAASVKGR | |
| | | FTISRDDSKSIAYLQMNSLKTEDTAVYYCSRDPWY | |
| | | CSGGDCYAVTGSWFDPWGQGTLVTVSS | |
| | VL | DIQMTQSPSSLSASVGDRVTITCQASQDIKQYLNW | 223 |
| | | YQQKPGKAPKLLIYDQSNLETGVPSRFSGSGSGTD | |
| | | FTFTISSLQPEDVAIYYCQQYWNLPQTFGQGTKVEI | |
| | | K | |
| A-24.3 | VH CDR1 | FTFADYTMS | 217 |
| | VH CDR2 | FIRSEPYGGTTEYAASVKG | 218 |
| | VH CDR3 | SRDPWYCSGGDCYAVTGSWFDP | 67 |
| | VL CDR1 | QASRDIKKYLN | 224 |
| | VL CDR2 | DVSNLET | 69 |
| | VL CDR3 | QQYDQLPQT | 225 |
| | VH | EVQLVESGGGLVKPGRSLRLSCAASGFTFADYTMS | 219 |
| | | WFRQAPGKGLEWVGFIRSEPYGGTTEYAASVKGR | |
| | | FTISRDDSKSIAYLQMNSLKTEDTAVYYCSRDPWY | |
| | | CSGGDCYAVTGSWFDPWGQGTLVTVSS | |
| | VL | DIQMTQSPSSLSASVGDRVTITCQASRDIKKYLNW | 226 |
| | | YQQKPGKAPKLLIYDVSNLETGVPSRFSGSGSGTD | |
| | | FTFTISSLQPEDVAIYYCQQYDQLPQTFGQGTKVEI | |
| | | E | |

TABLE 3
SARS-CoV-2 Variants Tested

| Variant | WHO | Old | |
|---|---|---|---|
| Name | Labels | Names | RBD Mutations |
| SARS CoV-2 | — | | |
| Wild Type | | | |
| SARS-CoV-2 | Alpha | UK | N501Y |
| B.1.1.7 | | | |
| N501Y | | | |
| SARS-CoV-2 | Beta | South | K417N, E484K, |
| B.1.351 | | African | N501Y |
| SARS-CoV-2 | Gamma | Brazilian | K417T, E484K, |
| P.1 | | | N501Y |
| SARS-CoV-2 | Delta | India | L452R, T478K |
| B.1.617.2 | | | |
| SARS CoV-2 | Epsilon | SoCal | L452R |
| B.1.427/429 | | | |
| SARS Cov-2 | Kappa | India | L452R, E484Q |
| B.1.617.1 | | | |
| SARS-CoV-2 | Iota | New York | E484K, S477N |
| B.1.526 | | | |
| SARS-CoV-2 | Omicron | | G339D, S371L, |
| B.1.529 | | | S373P, S375F, |
| | | | K417N, N440K, |
| | | | G446S, S477N, |
| | | | T478K, E484A, |
| | | | Q493R, G496S, |
| | | | Q498R, N501Y, |
| | | | Y505H |

TABLE 4
Select Antibodies Monovalent K_{D} Values (M) from Single Point Octet Binding

| | | | | B.1.1.7 | | | | L452R | |
|---|---|---|---|---|---|---|---|---|---|
| | Epitope | Ace2 | | N501Y | B.1.351 | P.1 Spike | B.1.617.2 | RBD | B.1.617.1 |
| Sample | Bin | Competitor | WT RBD | (Alpha) | (Beta) | (Gamma) | (Delta) | (Epsilon) | (Kappa) |
| A-6 | VHH-72 | Yes | 1.18E−09 | 1.46E−09 | 1.78E−09 | 2.05E−09 | Not | 1.45E−09 | Not Tested |
| | | | | | | | Tested | | |
| A-18 | VHH-72 | Yes | 5.27E−10 | 1.03E−09 | 4.44E−10 | 5.53E−10 | 6.49E−10 | 1.22E−09 | 7.14E−10 |
| A-22 | VHH-72 | Yes | 3.25E−10 | 3.50E−10 | 2.50E−10 | 2.58E−10 | 1.37E−10 | 7.56E−10 | 2.18E−10 |
| A-9 | RGN | Yes | 4.36E−09 | 1.00E−08 | 1.78E−08 | 1.50E−08 | 1.29E−08 | 1.24E−08 | 6.28E−09 |
| | 10933 | | | | | | | | |
| A-16 | RGN | No | 2.09E−09 | 4.62E−09 | 6.05E−09 | 4.83E−09 | Weak | Weak | Weak |
| | 10987 | | | | | | Binding | Binding | Binding |
| RGN | | Yes | 3.62E−09 | 6.57E−09 | Weak or | Weak or | 2.70E−09 | 9.52E−09 | 1.20E−08 |
| 10933 | | | | | non- | non- | | | |
| | | | | | specific | specific | | | |
| | | | | | binding | binding | | | |
| RGN | | No | 9.13E−09 | 1.51E−08 | 1.50E−08 | 1.25E−08 | 9.65E−09 | 1.74E−08 | 3.05E−09 |
| 10987 | | | | | | | | | |

TABLE 5
IC50 and IC90 values from Variant Pseudovirus
Neutralization Assays for Select antibodies
(Corresponds to FIGS. 4A-4F)

| [μg/ml] | | WT | Alpha | Beta | Epsilon | Gamma |
|---|---|---|---|---|---|---|
| A-6 | IC50 | 0.173 | 0.192 | 0.171 | | |
| | IC90 | 2.034 | 1.214 | 1.465 | | |
| A-18 | IC50 | 0.303 | 0.316 | 0.259 | | |
| | IC90 | 1.823 | 2.039 | 1.649 | | |
| A-22 | IC50 | 0.749 | 1.535 | 2.132 | 1.488 | 0.335 |
| | IC90 | 3.549 | 9.543 | 12.83 | 25.29 | 0.736 |
| A-9 | IC50 | 0.011 | 0.019 | 0.028 | | |
| | IC90 | 0.164 | 0.118 | 0.184 | | |
| A-16 | IC50 | 0.057 | 0.025 | 0.103 | | |
| | IC90 | 0.399 | 0.329 | 0.905 | | |
| REGN | IC50 | 0.0067 | 0.0064 | 0.897 | 0.0074 | 3.892 |
| 10933 | IC90 | 0.081 | 0.081 | 8.275 | 0.058 | 16.200 |

TABLE 6
IC50 and IC90 values from Coronavirus Family
Member Pseudovirus Neutralization Assays
for Select Antibodies
(Corresponds to FIG. 5)

| | [μg/ml] | SARS-1 | SARS-2 | WIV-6 |
|---|---|---|---|---|
| A-6 | IC50 | 1.014 | 0.015 | 0.074 |
| | IC90 | N/A | 0.085 | 16.290 |
| A-18 | IC50 | 0.043 | 0.076 | 0.045 |
| | IC90 | 0.046 | 1.381 | 0.324 |
| S309 | IC50 | 0.010 | 0.009 | 0.016 |
| | IC90 | 0.043 | 0.051 | 0.305 |
| RGN | IC50 | N/A | 0.002 | N/A |
| 10933 | IC90 | N/A | 0.024 | N/A |
| RGN | IC50 | N/A | 0.007 | N/A |
| 10987 | IC90 | N/A | 0.078 | N/A |

TABLE 7
Comparison of Live Virus IC50s to Pseudovirus IC50s
(Corresponds to FIG. 6)

| | Live Virus | PSV IC50 |
|---|---|---|
| Antibody | IC50 (μg/L) | (μg/L) |
| A-2 | 20 | 25 |
| A-11 | 29 | 92 |
| A-16 | 156 | 186 |
| A-10 | 71 | 194 |
| A-6 | 234 | 878 |
| A-18 | 234 | 1482 |
| A-1 | 21 | 17 |
| A-13 | 7 | 23 |
| A-18 | 120 | 242 |
| A-19 | 1220 | 5115 |
| RGN10933 | 12 | 28 |
| RGN10987 | 218 | 31 |
| RGN cocktail of 2 | 8 | 27 |

TABLE 8
Summary of Select Antibody Data

| | SARS2 | Beta | Delta | | | Live Virus | In vivo |
|---|---|---|---|---|---|---|---|
| | IC50 | IC50 | IC50 | Omicron | SARS1 | IC50 | Mouse Efficacy |
| Antibody | (M) | (M) | (M) | IC50 | IC50 | (M) | @ 0.5 mg/kg |
| A-6 | 2.3e−11 | 7.8e−11 | 2.1e−10 | Low | Strong | 1.6e−9 | ND |
| | 3.6e−11 | 5.3e−11 | 1.2e−10 | | ND | ND | ND |
| A-18 | 5.9e−9 | 4.5e−9 | 1.0e−8 | 10× lower | Strong | 1.6e−9 | ND |
| | <1e−11 | <1e−11 | <2e−11 | | ND | ND | ND |
| A-22 | 6.0e−11 | 7.5e−11 | 1.6e−10 | Low | Strong | ND | 10× weaker |
| | | | | | | | than |
| | | | | | | | RGN10933 |
| | 4.4e−11 | 3.4e−11 | 1.4e−10 | | ND | ND | ND |
| A-9 | 3.0e−11 | 1.2e−10 | 2.0e−10 | Low | NB | ND | Equivalent to |
| | | | | | | | RGN10933 |
| | 2.6e−11 | 2.7e−11 | 2.7e−11 | | ND | ND | ND |
| A-16 | 6.1e−10 | 7.3e−10 | 1.7e−9 | High = | NB | 1.0e−9 | Equivalent to |
| | | | | WT IC50 | | | RGN10933 |
| | 6.7c−11 | 3.7c−10 | 4.3c−10 | | ND | TBD | ND |
| RGN | 2.2e−11 | 2.9e−8 | 4.1e−11 | NB | NB | 7.8e−11 | >50% viral |
| 10933 | | | | | | | reduction |
| S309 | TBD | TBD | TBD | High = | NB | 3.5e−9* | ND |
| | | | | WT IC50 | | | |

TABLE 9
Independent Testing Confirming Select antibodies have
Activity against Omicron
(Corresponds to FIG. 18)

| IC50 | Test 1: | Test 1: | Test 2: | Test 2: |
|---|---|---|---|---|
| (nM) | Wuhan WT | Omicron | Wuhan WT | Omicron |
| A-6 | 0.12 | X* | 2.45 | X* |
| A-18 | 0.14 | 1.96 | 2.78 | 8.84 |
| A-22 | X* | X* | No Fit | No Fit |
| A-16 | 0.28 | 0.19 | 0.54 | 2.68 |
| A-9 | 0.03 | 20.76 | 0.04 | No Fit |

*“X” indicates poor fit or incomplete neutralization

TABLE 10
Affinity Maturation Data

| | | | | | Octet IgG K_{D} | | | | | | | |
|---|---|---|---|---|---|---|---|---|---|---|---|---|
| | | | Octet IgG K_{D} | Octet IgG K_{D} | SARS-CoV-2 | Octet IgG K_{D} | | | Octet Fab K_{D} | | | |
| | Octet IgG K_{D} | Octet IgG K_{D} | SARS-CoV-2 | SARS-CoV-2 | Variant | SARS-COV-2 | Octet IgG K_{D} | Octet Fab K_{D} | SARS-CoV-2 | | | |
| | SARS-CoV-2 | SARS-CoV-2 | Variant | Variant | B.1.17 | Variant | SARS-CoV1 | SARS-CoV-2 | Variant | | | |
| | Spike | Variant P.1 | L452R | B.1.351 | N501Y S1- | L452R_T478K | Spike | Spike | L452R_E484Q | | | |
| | S1-His (M) | S1-His (M) | S1-His (M) | S1-His (M) | His (M) | S1-His (M) | S1-His (M) | S1-His (M) | S1-His (M) | WT PsV | Delta PsV | Omicron PsV |
| Identifier | Monovalent | Monovalent | Monovalent | Monovalent | Monovalent | Monovalent | Monovalent | Monovalent | Monovalent | IC50 [M] | IC50 [M] | IC50 [M] |
| A-20 | 3.95E−09 | 3.02E−09 | 1.82E−09 | 2.96E−09 | 2.57E−09 | 1.79E−09 | 2.20E−08 | N.D. | N.D. | 2.454E−09 | 7.461E−09 | 7.382E−08 |
| A-20.1 | 8.75E−10 | 8.67E−10 | 3.61E−10 | 8.29E−10 | 6.04E−10 | 4.23E−10 | 5.29E−09 | N.D. | N.D. | 8.974E−10 | 1.017E−09 | 2.395E−08 |
| A-20.2 | 2.93E−10 | 2.43E−10 | 1.96E−10 | 2.30E−10 | 2.10E−10 | 1.78E−10 | 1.74E−09 | N.D. | N.D. | 1.058E−09 | 9.856E−10 | 8.617E−09 |
| A-20.3 | 3.66E−10 | 2.89E−10 | 1.77E−10 | 2.60E−10 | 2.44E−10 | 1.81E−10 | 3.52E−09 | N.D. | N.D. | 1.506E−09 | 1.685E−09 | 3.462E−09 |
| A-21 | 5.29E−10 | 2.43E−10 | 3.62E−10 | 2.95E−10 | 4.11E−10 | 4.06E−10 | 9.03E−09 | N.D. | N.D. | 2.784E−09 | 4.157E−09 | 8.841E−09 |
| A-21.1 | 4.61E−10 | 2.48E−10 | 3.46E−10 | 2.73E−10 | 3.97E−10 | 4.27E−10 | 6.57E−09 | N.D. | N.D. | 1.748E−09 | 1.43E−09 | 8.754E−09 |
| A-21.2 | 1.95E−10 | 1.15E−10 | 1.40E−10 | 1.16E−10 | 1.28E−10 | 1.24E−10 | 7.93E−09 | N.D. | N.D. | 2.634E−09 | 4.172E−09 | 4.339E−09 |
| A-21.3 | 2.41E−10 | 1.51E−10 | 1.70E−10 | 1.46E−10 | 1.61E−10 | 1.57E−10 | 8.93E−10 | N.D. | N.D. | 9.455E−10 | 1.245E−09 | 6.601E−09 |
| A-22 | 2.44E−10 | 1.61E−10 | 1.58E−10 | 1.35E−10 | 1.53E−10 | 1.42E−10 | 4.51E−09 | N.D. | N.D. | 2.878E−08 | 4.923E−07 | 5.302E−08 |
| A-22.1 | 2.78E−10 | 1.99E−10 | 1.84E−10 | 1.67E−10 | 1.83E−10 | 1.66E−10 | 3.68E−09 | N.D. | N.D. | 1.428E−08 | 9.193E−09 | 5.318E−08 |
| A-22.2 | 2.54E−10 | 1.80E−10 | 1.69E−10 | 1.51E−10 | 1.62E−10 | 1.55E−10 | 1.01E−08 | N.D. | N.D. | 3.299E−09 | 3.504E−09 | 7.433E−08 |
| A-22.3 | 2.45E−10 | 1.71E−10 | 1.64E−10 | 1.51E−10 | 1.58E−10 | 1.51E−10 | 4.77E−09 | N.D. | N.D. | 7.098E−09 | 4.574E−09 | 7.933E−08 |
| A-23 | 2.11E−09 | 2.54E−09 | P.F. | 2.93E−09 | 1.20E−09 | P.F. | N.B. | N.D. | N.D. | 5.392E−10 | 4.529E−09 | 2.681E−09 |
| A-23.1 | 8.29E−10 | 1.09E−09 | 6.21E−08 | 1.19E−09 | 5.12E−10 | 7.79E−08 | N.B. | N.D. | N.D. | 4.231E−10 | 1.721E−09 | 1.361E−09 |
| A-23.2 | 4.54E−10 | 5.50E−10 | 3.99E−08 | 6.53E−10 | 2.42E−10 | 4.76E−08 | N.B. | N.D. | N.D. | 4.156E−10 | 3.672E−09 | 7.861E−10 |
| A-23.3 | 5.21E−10 | 5.10E−10 | 3.83E−08 | 6.34E−10 | 2.86E−10 | 4.04E−08 | N.B. | N.D. | N.D. | 4.973E−10 | 1.767E−08 | 1.179E−09 |
| A-24 | 5.36E−09 | 8.30E−09 | 4.51E−09 | 7.59E−09 | 3.84E−09 | 3.66E−08 | N.B. | 2.97E−09 | 3.76E−09 | 3.905E−11 | 3.786E−10 | 1.872E−10 |
| A-24.1 | 9.99E−10 | 2.68E−09 | 9.15E−10 | 2.52E−09 | 8.89E−10 | 2.37E−09 | N.B. | 5.39E−10 | 7.07E−10 | 6.569E−11 | 3.721E−11 | 2.301E−09 |
| A-24.2 | 2.89E−10 | 1.01E−09 | 2.55E−10 | 6.55E−10 | 2.37E−10 | 4.38E−10 | N.B. | N.D. | N.D. | 1.157E−10 | 3.97E−11 | 6.794E−11 |
| A-24.3 | 4.79E−10 | 1.40E−09 | 3.87E−10 | 1.10E−09 | 3.88E−10 | 1.33E−09 | N.B. | 2.44E−10 | 3.16E−10 | 4.541E−11 | 3.557E−11 | 2.548E−10 |
| RGN10933 | | | | | | | | | | 1.851E−10 | 2.897E−10 | 3.249E−08 |
| RGN10987 | | | | | | | | | | 2.922E−10 | 5.226E−10 | 2.416E−10 |
| S309 | | | | | | | | | | 6.556E−09 | 5.685E−11 | 1.658E−08 |

Equilibrium dissociation constant (KD = koff/kon). Data fit to a 1:1 binding model.
P.F. Poor Fit. Binding under the conditions of this assay but unable to be adequately fit to a 1:1 binding model.
N.B. = No observable binding under the conditions of this assay. Unable to assign a KD.
Reached koff limit for 600 second dissociation
Lineage Parent

TABLE 11
PBMC data

| | | | | Octet IgG | Octet IgG | Octet IgG | Octet IgG | Octet IgG | | |
|---|---|---|---|---|---|---|---|---|---|---|
| | | | | KD SARS- | KD SARS- | KD SARS- | KD SARS- | KD MERS- | | |
| | | Bin | | CoV-2 S | CoV-2 | CoV-2 S1- | CoV S1-His | CoV S1-His | PsV Neut | PsV Neut |
| TAK | | Code | ACE2 | Trimer (M) | RBD-mFc | His (M) | (M) | (M) | Assay | Assay |
| Name | Selection Antigen | (see FIG. 2) | Competitor | Avid | (M) Avid | Monovalent | Monovalent | Monovalent | IC50 [M] | IC50 [M] |
| A-1 | SARS-COV-2 Trimer | 2, 4 | Yes | 8.65E−10 | 8.90E−11 | 9.60E−09 | N.B. | N.B. | 1.16E−10 | legit |
| A-2 | SARS-COV-2 Trimer | 2 | Yes | 1.14E−09 | 1.25E−10 | 1.45E−08 | N.B. | N.B. | 1.69E−10 | legit |
| A-3 | SARS-COV-2 RBD- | 2 | Yes | 9.45E−10 | 1.41E−10 | 3.74E−08 | N.B. | N.B. | 4.19E−10 | legit |
| | mFC | | | | | | | | | |
| A-4 | SARS-COV-2 RBD- | 2 | Yes | 1.97E−09 | 6.63E−10 | N.B. | N.B. | N.B. | 2.26E−09 | legit |
| | mFC | | | | | | | | | |
| A-5 | SARS-COV-2 RBD- | 2 | Yes | 2.41E−09 | 1.79E−10 | 3.73E−08 | N.B. | N.B. | 1.10E−09 | legit |
| | mFC | | | | | | | | | |
| A-6 | SARS-COV-2 Trimer | 1 | Yes | 1.35E−09 | 1.46E−10 | 3.71E−09 | 1.72E−08 | N.B. | 5.85E−09 | legit |
| A-7 | SARS-COV-2 Trimer | 2 | Yes | 8.04E−10 | 9.41E−11 | 2.71E−09 | N.B. | N.B. | 9.22E−11 | legit |
| A-8 | SARS-COV-2 Trimer | 2 | Yes | 1.53E−09 | 1.06E−10 | 4.77E−10 | N.B. | N.B. | 8.46E−10 | legit |
| A-9 | SARS-COV-2 Trimer | 2 | Yes | 1.00E−09 | 1.47E−10 | 5.89E−09 | N.B. | N.B. | 2.08E−10 | legit |
| A-10 | SARS-COV-2 Trimer | Other | No | 8.54E−10 | 9.31E−11 | 9.59E−09 | N.B. | N.B. | 1.29E−09 | legit |
| A-11 | SARS-COV-2 Trimer | Other | Yes | 1.29E−09 | 1.73E−10 | 6.92E−09 | N.B. | N.B. | 6.13E−10 | legit |
| A-12 | SARS-COV-2 RBD- | 2 | Yes | 1.11E−09 | 1.51E−10 | 2.57E−09 | N.B. | N.B. | 4.04E−10 | legit |
| | mFC | | | | | | | | | |
| A-13 | SARS-COV-2 RBD- | 2 | Yes | 8.43E−10 | 9.59E−11 | 4.91E−09 | N.B. | N.B. | 1.55E−10 | legit |
| | mFC | | | | | | | | | |
| A-14 | SARS-COV-2 RBD- | 2 | Yes | 2.09E−09 | 1.58E−10 | 2.66E−08 | N.B. | N.B. | 1.61E−09 | legit |
| | mFC | | | | | | | | | |
| A-15 | SARS-COV-2 RBD- | 2 | Yes | 1.62E−09 | 2.12E−10 | 7.82E−09 | N.B. | N.B. | 1.44E−09 | legit |
| | mFC | | | | | | | | | |
| A-16 | SARS-COV-2 RBD- | 4 | No | 1.15E−09 | 8.10E−11 | 4.85E−09 | N.B. | N.B. | 1.24E−09 | legit |
| | mFC | | | | | | | | | |
| A-17 | SARS-COV-2 RBD- | 2 | Yes | 8.85E−10 | 1.31E−10 | 1.58E−09 | N.B. | N.B. | 6.60E−10 | legit |
| | mFC | | | | | | | | | |
| A-18 | SARS-COV-2 RBD- | 2 | Yes | 1.69E−09 | 7.57E−11 | 1.09E−09 | 7.43E−09 | N.B. | 9.88E−09 | legit |
| | mFC | | | | | | | | | |
| A-19 | SARS-COV-2 RBD- | 4 | No | 9.87E−10 | 1.29E−10 | 1.15E−09 | N.B. | N.B. | 3.41E−08 | legit |
| | mFC | | | | | | | | | |

N.B. = Non-Binder under the conditions of this assay
Reached koff limit for 300 or 600 second dissociation

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

Various terms relating to aspects of disclosure are used throughout the specification and claims. Such terms are to be given their ordinary meaning in the art, unless otherwise indicated. Other specifically defined terms are to be construed in a manner consistent with the definition provided herein.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. An antibody that binds to a severe acute respiratory syndrome coronavirus 2 (SARS-CoV-2) spike protein, wherein the antibody comprises:
a heavy chain variable domain (VH) comprising a heavy chain complementary determining region 1 (CDR-H1) comprising the amino acid sequence of FTFX₁NFAMH (SEQ ID NO: 230), wherein X₁ is S, T, D or E; a heavy chain complementary determining region 2 (CDR-H2) comprising the amino acid sequence of VIX₂YDGINX₃YYADSVKG (SEQ ID NO: 231), wherein X₂ is A, L, I, V, E, or D, and X₃ is K, R, D or E; a heavy chain complementary determining region 3 (CDR-H3) comprising the amino acid sequence of ARAQNYYDRX₄GTLQLDAFDI (SEQ ID NO: 232), wherein X₄ is S, T, Y, H; and/or
a light chain variable domain (VL) comprising a light chain complementary determining region 1 (CDR-L1) comprising the amino acid sequence of X₅ASX₆GIRNDLG (SEQ ID NO: 233), wherein X₅ is R, K, V, L, A, or I and X₆ is Q, N, E or D; a light chain complementary determining region 1 (CDR-L2) comprising the amino acid sequence of X₇ASSLX₈S (SEQ ID NO: 234), wherein X₇ is A, V, L, I, E or D, and X₈ is Q, N, D or E; and a light chain complementary determining region 1 (CDR-L3) comprising the amino acid sequence of LQX₉YNYPLT (SEQ ID NO: 235), wherein X₉ is N, Q, E or D.

2. The antibody of claim 1, wherein:
the CDR-H1 comprises the amino acid sequence of FTFX₁NFAMH (SEQ ID NO: 236), wherein X₁ is S or D; the CDR-H2 comprises the amino acid sequence of VIX₂YDGINX₃YYADSVKG (SEQ ID NO: 237), wherein X₂ is L or E, and X₃ is K or D;
the CDR-H3 comprises the amino acid sequence of ARAQNYYDRX₄GTLQLDAFDI (SEQ ID NO: 238), wherein X₄ is S, Y, or H; and/or
the CDR-L1 comprises the amino acid sequence of X₅ASX₆GIRNDLG (SEQ ID NO: 239), wherein X₅ is R or V and X₆ is Q or D; the CDR-L2 comprises the amino acid sequence of X₇ASSLX₈S (SEQ ID NO: 240), wherein X₇ is A, E or D, and X₈ is Q or E; and the CDR-L3 comprises the amino acid sequence of LQX₉YNYPLT (SEQ ID NO: 241), wherein X₉ is N or D.

3. The antibody of claim 1 or claim 2, wherein:
the CDR-H1 comprises the amino acid sequence of SEQ ID NO: 41, the CDR-H2 comprises the amino acid sequence of SEQ ID NO: 42, the CDR-H3 comprises the amino acid sequence of SEQ ID NO: 43; and/or
the CDR-L1 comprises the amino acid sequence of SEQ ID NO: 44, the CDR-L2 comprises the amino acid sequence of SEQ ID NO: 45, and the CDR-L3 comprises the amino acid sequence of SEQ ID NO: 46.

4. The antibody of claim 3, wherein the VH comprises the amino acid sequence of SEQ ID NO: 149, and the VL comprises the amino acid sequence of SEQ ID NO: 150.

5. The antibody of claim 3, wherein the VH comprises the amino acid sequence of SEQ ID NO: 47, and the VL comprises the amino acid sequence of SEQ ID NO: 48.

6. The antibody of claim 1 or claim 2, wherein:
the CDR-H1 comprises the amino acid sequence of SEQ ID NO: 41, the CDR-H2 comprises the amino acid sequence of SEQ ID NO: 42, the CDR-H3 comprises the amino acid sequence of SEQ ID NO: 43; and/or
the CDR-L1 comprises the amino acid sequence of SEQ ID NO: 151, the CDR-L2 comprises the amino acid sequence of SEQ ID NO: 152, and the CDR-L3 comprises the amino acid sequence of SEQ ID NO: 46.

7. The antibody of claim 6, wherein the VH comprises the amino acid sequence of SEQ ID NO: 149, and the VL comprises the amino acid sequence of SEQ ID NO: 153.

8. The antibody of claim 1 or claim 2, wherein:
the CDR-H1 comprises the amino acid sequence of SEQ ID NO: 154, the CDR-H2 comprises the amino acid sequence of SEQ ID NO: 155, the CDR-H3 comprises the amino acid sequence of SEQ ID NO: 156; and/or
the CDR-L1 comprises the amino acid sequence of SEQ ID NO: 44, the CDR-L2 comprises the amino acid sequence of SEQ ID NO: 157, and the CDR-L3 comprises the amino acid sequence of SEQ ID NO: 46.

9. The antibody of claim 8, wherein the VH comprises the amino acid sequence of SEQ ID NO: 158, and the VL comprises the amino acid sequence of SEQ ID NO: 159.

10. The antibody of claim 1 or claim 2, wherein:
the CDR-H1 comprises the amino acid sequence of SEQ ID NO: 154, the CDR-H2 comprises the amino acid sequence of SEQ ID NO: 160, the CDR-H3 comprises the amino acid sequence of SEQ ID NO: 161; and/or
the CDR-L1 comprises the amino acid sequence of SEQ ID NO: 162, the CDR-L2 comprises the amino acid sequence of SEQ ID NO: 163, and the CDR-L3 comprises the amino acid sequence of SEQ ID NO: 164.

11. The antibody of claim 10, wherein the VH comprises the amino acid sequence of SEQ ID NO: 165, and the VL comprises the amino acid sequence of SEQ ID NO: 166.

12. An antibody that binds to a SARS-CoV-2 spike protein, wherein the antibody comprises:
a VH comprising a CDR-H1 comprising the amino acid sequence of FTFX₁RYWMH (SEQ ID NO: 242), wherein X₁ is S, T, K or R; a CDR-H2 comprising the amino acid sequence of RINSDGSSTTYADSVX₂G (SEQ ID NO: 243), wherein X₂ is K, R, D or E; a CDR-H3 comprising the amino acid sequence of ARELYYYDX₃RGX₄EEGEGWFDP (SEQ ID NO: 244), wherein X₃ is S, T, K and R, and X₄ is A, V, I, L, W or F; and/or
a VL comprising a CDR-L1 comprising the amino acid sequence of TGISSNIGAX₅YDVH, wherein X₅ is G, N, Q, D, or E (SEQ ID NO: 245); a CDR-L2 comprising the amino acid sequence of GNSNRPX₆ (SEQ ID NO: 246), wherein X₆ is S, T, K or R; and a CDR-L3 comprising the amino acid sequence of QX₇SDRX₈X₉SGSV (SEQ ID NO: 247), wherein X₇ is S, T, A, I, L or V, and X₈ is S, T, or Y, and X₉ is L, I, or V.

13. The antibody of claim 12, wherein:
the CDR-H1 comprises the amino acid sequence of FTFX₁RYWMH (SEQ ID NO: 248), wherein X₁ is S or R; the CDR-H2 comprises the amino acid sequence of RINSDGSSTTYADSVX₂G (SEQ ID NO: 249), wherein X₂ is K or E; the CDR-H3 comprises the amino acid sequence of ARELYYYDX₃RGX₄EEGEGWFDP (SEQ ID NO: 250), wherein X₃ is S or R, and X₄ is A or F; and/or
the CDR-L1 comprises the amino acid sequence of TGISSNIGAX₅YDVH (SEQ ID NO: 251), wherein X₅ is G, Q, or E; the CDR-L2 comprises the amino acid sequence of GNSNRPX₆ (SEQ ID NO: 252), wherein X₆ is S or R; and the CDR-L3 comprises the amino acid sequence of QX₇SDRX₈X₉SGSV (SEQ ID NO: 253), wherein X₇ is S or V, and X₈ is S or Y, and X₉ is L or V.

14. The antibody of claim 12 or claim 13, wherein:
the CDR-H1 comprises the amino acid sequence of SEQ ID NO: 133, the CDR-H2 comprises the amino acid sequence of SEQ ID NO: 134, the CDR-H3 comprises the amino acid sequence of SEQ ID NO: 135; and/or
the CDR-L1 comprises the amino acid sequence of SEQ ID NO: 136, the CDR-L2 comprises the amino acid sequence of SEQ ID NO: 137, and the CDR-L3 comprises the amino acid sequence of SEQ ID NO: 138.

15. The antibody of claim 14, wherein the VH comprises the amino acid sequence of SEQ ID NO: 167, and the VL comprises the amino acid sequence of SEQ ID NO: 168.

16. The antibody of claim 14, wherein the VH comprises the amino acid sequence of SEQ ID NO: 139, and the VL comprises the amino acid sequence of SEQ ID NO: 140.

17. The antibody of claim 12 or claim 13, wherein:
the CDR-H1 comprises the amino acid sequence of SEQ ID NO: 169, the CDR-H2 comprises the amino acid sequence of SEQ ID NO: 134, the CDR-H3 comprises the amino acid sequence of SEQ ID NO: 170; and/or
the CDR-L1 comprises the amino acid sequence of SEQ ID NO: 136, the CDR-L2 comprises the amino acid sequence of SEQ ID NO: 137, and the CDR-L3 comprises the amino acid sequence of SEQ ID NO: 138.

18. The antibody of claim 17, wherein the VH comprises the amino acid sequence of SEQ ID NO: 171, and the VL comprises the amino acid sequence of SEQ ID NO: 168.

19. The antibody of claim 12 or claim 13, wherein:
the CDR-H1 comprises the amino acid sequence of SEQ ID NO: 133, the CDR-H2 comprises the amino acid sequence of SEQ ID NO: 134, the CDR-H3 comprises the amino acid sequence of SEQ ID NO: 135; and/or
the CDR-L1 comprises the amino acid sequence of SEQ ID NO: 172, the CDR-L2 comprises the amino acid sequence of SEQ ID NO: 173, and the CDR-L3 comprises the amino acid sequence of SEQ ID NO: 174.

20. The antibody of claim 19, wherein the VH comprises the amino acid sequence of SEQ ID NO: 167, and the VL comprises the amino acid sequence of SEQ ID NO: 175.

21. The antibody of claim 12 or claim 13, wherein:
the CDR-H1 comprises the amino acid sequence of SEQ ID NO: 169, the CDR-H2 comprises the amino acid sequence of SEQ ID NO: 176, the CDR-H3 comprises the amino acid sequence of SEQ ID NO: 177; and/or
the CDR-L1 comprises the amino acid sequence of SEQ ID NO: 178, the CDR-L2 comprises the amino acid sequence of SEQ ID NO: 173, and the CDR-L3 comprises the amino acid sequence of SEQ ID NO: 179.

22. The antibody of claim 21, wherein the VH comprises the amino acid sequence of SEQ ID NO: 180, and the VL comprises the amino acid sequence of SEQ ID NO: 181.

23. An antibody that binds to a SARS-CoV-2 spike protein, wherein the antibody comprises:
a VH comprising a CDR-H1 comprising the amino acid sequence of X₁TFSSX₂DMS (SEQ ID NO: 254), wherein X₁ is F, W or P, and X₂ is Y, T or S; a CDR-H2 comprising the amino acid sequence of GISGX₃GAITYYTDSVKG (SEQ ID NO: 255), wherein X₃ is S, T, D or E; a CDR-H3 comprising the amino acid sequence of TKENNRX₄TFFDY (SEQ ID NO: 256), wherein X₄ is N, Q, A, L, I, V, or G; and/or
a VL comprising a CDR-LT comprising the amino acid sequence of X₅ASQTVRSDLA (SEQ ID NO: 257), wherein X₅ is R, K, S or T; a CDR-L2 comprising the amino acid sequence of GX₆STRAT (SEQ ID NO: 258), wherein X₆ is A, V, L, I or G; and a CDR-L3 comprising the amino acid sequence of QQYYEWPPHSDS (SEQ ID NO: 186).

24. The antibody of claim 23, wherein:
the CDR-H1 comprises the amino acid sequence of X₁TFSSX₂DMS (SEQ ID NO: 259), wherein X₁ is F or P, and X₂ is Y or T; the CDR-H2 comprises the amino acid sequence of GISGX₃GAITYYTDSVKG (SEQ ID NO: 260), wherein X₃ is S or E; the CDR-H3 comprises the amino acid sequence of TKENNRX₄TFFDY (SEQ ID NO: 261), wherein X₄ is N, A, or G; and/or the CDR-L1 comprises the amino acid sequence of X₅ASQTVRSDLA (SEQ ID NO: 262), wherein X₅ is R or S; the CDR-L2 comprises the amino acid sequence of GX₆STRAT (SEQ ID NO: 263), wherein X₆ is A or G; and the CDR-L3 comprises the amino acid sequence of QQYYEWPPHSDS (SEQ ID NO: 186).

25. The antibody of claim 23 or claim 24, wherein:
the CDR-H1 comprises the amino acid sequence of SEQ ID NO: 182, the CDR-H2 comprises the amino acid sequence of SEQ ID NO: 183, the CDR-H3 comprises the amino acid sequence of SEQ ID NO: 184; and/or
the CDR-L1 comprises the amino acid sequence of SEQ ID NO: 185, the CDR-L2 comprises the amino acid sequence of SEQ ID NO: 77, and the CDR-L3 comprises the amino acid sequence of SEQ ID NO: 186.

26. The antibody of claim 25, wherein the VH comprises the amino acid sequence of SEQ ID NO: 187, and the VL comprises the amino acid sequence of SEQ ID NO: 188.

27. The antibody of claim 23 or claim 24, wherein:
the CDR-H1 comprises the amino acid sequence of SEQ ID NO: 189, the CDR-H2 comprises the amino acid sequence of SEQ ID NO: 183, the CDR-H3 comprises the amino acid sequence of SEQ ID NO: 190; and/or
the CDR-L1 comprises the amino acid sequence of SEQ ID NO: 185, the CDR-L2 comprises the amino acid sequence of SEQ ID NO: 77, and the CDR-L3 comprises the amino acid sequence of SEQ ID NO: 186.

28. The antibody of claim 27, wherein the VH comprises the amino acid sequence of SEQ ID NO: 191, and the VL comprising the amino acid sequence of SEQ ID NO: 188.

29. The antibody of claim 23 or claim 24, wherein:
the CDR-H1 comprises the amino acid sequence of SEQ ID NO: 192, the CDR-H2 comprises the amino acid sequence of SEQ ID NO: 183, the CDR-H3 comprises the amino acid sequence of SEQ ID NO: 193; and/or
the CDR-L1 comprises the amino acid sequence of SEQ ID NO: 194, the CDR-L2 comprises the amino acid sequence of SEQ ID NO: 195, and the CDR-L3 comprises the amino acid sequence of SEQ ID NO: 186.

30. The antibody of claim 29, wherein the VH comprises the amino acid sequence of SEQ ID NO: 196, and the VL comprises the amino acid sequence of SEQ ID NO: 197.

31. The antibody of claim 23 or claim 24, wherein:
the CDR-H1 comprises the amino acid sequence of SEQ ID NO: 182, the CDR-H2 comprises the amino acid sequence of SEQ ID NO: 198, the CDR-H3 comprises the amino acid sequence of SEQ ID NO: 190; and/or
the CDR-L1 comprises the amino acid sequence of SEQ ID NO: 185, the CDR-L2 comprises the amino acid sequence of SEQ ID NO: 195, and the CDR-L3 comprises the amino acid sequence of SEQ ID NO: 186.

32. The antibody of claim 31, wherein the VH comprises the amino acid sequence of SEQ ID NO: 199, and the VL comprises the amino acid sequence of SEQ ID NO: 200.

33. An antibody that binds to a SARS-CoV-2 spike protein, wherein the antibody comprises:
a VH comprising a CDR-H1 comprising the amino acid sequence of FTX₁DDYAMH (SEQ ID NO: 264), wherein X₁ is F, W, I, or L; a CDR-H2 comprising the amino acid sequence of GX₂SWNX₃GTIGYADSVKG (SEQ ID NO: 265), wherein X₂ is I, L, V, A, S or T, and X₃ is S, T or G; comprising CDR-H3 comprising the amino acid sequence of X₄KDRKREDPSLGGMDV (SEQ ID NO: 266), wherein X₄ is G T, L, A or V; and/or
a VL comprising a CDR-L1 comprising the amino acid sequence of X₅GNNIGSKSVH (SEQ ID NO: 267), wherein X₅ is G, L, I, A or V; a CDR-L2 comprising the amino acid sequence of DDTDRPS (SEQ ID NO: 123); and a CDR-L3 comprising the amino acid sequence of QVWDX₆X₇SDX₈YV (SEQ ID NO: 268), wherein X₆ is S, T, I, L, A or V, and X₇ is S, T, D or E, and X₈ is H, S, T, E or D.

34. The antibody of claim 33, wherein:
the CDR-H1 comprises the amino acid sequence of FTX₁DDYAMH (SEQ ID NO: 269), wherein X₁ is F or I; the CDR-H2 comprises the amino acid sequence of GX₂SWNX₃GTIGYADSVKG (SEQ ID NO: 270), wherein X₂ is I or T, and X₃ is S or G;
the CDR-H3 comprises the amino acid sequence of X₄KDRKREDPSLGGMDV (SEQ ID NO: 271), wherein X₄ is G or V; and/or
the CDR-L1 comprises the amino acid sequence of X₅GNNIGSKSVH (SEQ ID NO: 272), wherein X₅ is G or L; the CDR-L2 comprises the amino acid sequence of DDTDRPS (SEQ ID NO: 123); and the CDR-L3 comprises the amino acid sequence of QVWDX₆X₇SDX₈YV (SEQ ID NO: 273), wherein X₆ is S or V, and X₇ is S or E, and X₈ is H, S, or D.

35. The antibody of claim 33 or claim 34, wherein:
the CDR-H1 comprises the amino acid sequence of SEQ ID NO: 119, the CDR-H2 comprises the amino acid sequence of SEQ ID NO: 120, the CDR-H3 comprises the amino acid sequence of SEQ ID NO: 121; and/or
the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 124.

36. The antibody of claim 35, wherein the VH comprises the amino acid sequence of SEQ ID NO: 201, and the VL comprises the amino acid sequence of SEQ ID NO: 202.

37. The antibody of claim 35, wherein the VH comprises the amino acid sequence of SEQ ID NO: 125, and the VL comprises the amino acid sequence of SEQ ID NO: 126.

38. The antibody of claim 33 or claim 34, wherein:
the CDR-H1 comprises the amino acid sequence of SEQ ID NO: 203, the CDR-H2 comprises the amino acid sequence of SEQ ID NO: 204, the CDR-H3 comprises the amino acid sequence of SEQ ID NO: 121; and/or
the CDR-L1 comprises the amino acid sequence of SEQ ID NO: 205, the CDR-L2 comprises the amino acid sequence of SEQ ID NO: 123, and the CDR-L3 comprises the amino acid sequence of SEQ ID NO: 206.

39. The antibody of claim 38, wherein the VH comprises the amino acid sequence of SEQ ID NO: 207, and the VL comprises the amino acid sequence of SEQ ID NO: 208.

40. The antibody of claim 33 or claim 34, wherein:
the CDR-H1 comprises the amino acid sequence of SEQ ID NO: 119, the CDR-H2 comprises the amino acid sequence of SEQ ID NO: 209, the CDR-H3 comprises the amino acid sequence of SEQ ID NO: 210; and/or
the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 211.

41. The antibody of claim 40, wherein the VH comprises the amino acid sequence of SEQ ID NO: 212, and the VL comprises the amino acid sequence of SEQ ID NO: 213.

42. The antibody of claim 33 or claim 34, wherein:
the CDR-H1 comprises the amino acid sequence of SEQ ID NO: 119, the CDR-H2 comprises the amino acid sequence of SEQ ID NO: 209, the CDR-H3 comprises the amino acid sequence of SEQ ID NO: 210; and/or
the CDR-L1 comprises the amino acid sequence of SEQ ID NO: 122, the CDR-L2 comprises the amino acid sequence of SEQ ID NO: 123, and the CDR-L3 comprises the amino acid sequence of SEQ ID NO: 214.

43. The antibody of claim 42, wherein the VH comprises the amino acid sequence of SEQ ID NO: 212, and the VL comprises the amino acid sequence of SEQ ID NO: 215.

44. An antibody that binds to a SARS-CoV-2 spike protein, wherein the antibody comprises:
a VH comprising a CDR-H1 comprising the amino acid sequence of FTFADYX₁MS (SEQ ID NO: 274), wherein X₁ is A, V, S or T; a CDR-H2 comprising the amino acid sequence of FIRSX₂PYGGTTEYAASVKG (SEQ ID NO: 275), wherein X₂ is K, R, E, and D; a CDR-H3 comprising the amino acid sequence of SRDPWYCSGGDCYAVTGSWFDP (SEQ ID NO: 67); and/or
a VL comprising a CDR-L1 comprising the amino acid sequence of QASX₃DIKX₄YLN (SEQ ID NO: 276), wherein X₃ is Q, N, K or R and X₄ is K, R, N, or Q; a CDR-L2 comprising the amino acid sequence of DX₅SNLET (SEQ ID NO: 277), wherein X₅ is V, I, L, A, N or Q; and a CDR-L3 comprising the amino acid sequence of QQYX₆X₇LPQT (SEQ ID NO: 278), wherein X₆ is D, E, F or W, and X₇ is N or Q.

45. The antibody of claim 44, wherein:
the CDR-H1 comprises the amino acid sequence of FTFADYX₁MS (SEQ ID NO: 279), wherein X₁ is A or T; the CDR-H2 comprises the amino acid sequence of FIRSX₂PYGGTTEYAASVKG (SEQ ID NO: 280), wherein X₂ is K or E; the CDR-H3 comprises the amino acid sequence of SRDPWYCSGGDCYAVTGSWFDP (SEQ ID NO: 67); and/or
the CDR-L1 comprises the amino acid sequence of QASX₃DIKX₄YLN (SEQ ID NO: 281), wherein X₃ is Q or R and X₄ is K or Q; the CDR-L2 comprises the amino acid sequence of DX₅SNLET (SEQ ID NO: 282), wherein X₅ is V or Q; and the CDR-L3 comprises the amino acid sequence of QQYX₆X₇LPQT (SEQ ID NO: 283), wherein X₆ is D or W, and X₇ is N or Q.

46. The antibody of claim 44 or claim 45, wherein:
the CDR-H1 comprises the amino acid sequence of SEQ ID NO: 65, the CDR-H2 comprises the amino acid sequence of SEQ ID NO: 66, the CDR-H3 comprises the amino acid sequence of SEQ ID NO: 67; and/or
the CDR-L1 comprises the amino acid sequence of SEQ ID NO: 68, the CDR-L2 comprises the amino acid sequence of SEQ ID NO: 69, and the CDR-L3 comprises the amino acid sequence of SEQ ID NO: 70.

47. The antibody of claim 46, wherein the VH comprises the amino acid sequence of SEQ ID NO: 71, and the VL comprises the amino acid sequence of SEQ ID NO: 216.

48. The antibody of claim 46, wherein the VH comprises the amino acid sequence of SEQ ID NO: 71, and the VL comprises the amino acid sequence of SEQ ID NO: 72.

49. The antibody of claim 44 or claim 45, wherein:
the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or
the CDR-L1 comprises the amino acid sequence of SEQ ID NO: 68, the CDR-L2 comprises the amino acid sequence of SEQ ID NO: 69, and the CDR-L3 comprises the amino acid sequence of SEQ ID NO: 70.

50. The antibody of claim 49, wherein the VH comprises the amino acid sequence of SEQ ID NO: 219, and the VL comprises the amino acid sequence of SEQ ID NO: 216.

51. The antibody of claim 44 or claim 45, wherein:
the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or
the CDR-L1 comprises the amino acid sequence of SEQ ID NO: 220, the CDR-L2 comprises the amino acid sequence of SEQ ID NO: 221, and the CDR-L3 comprises the amino acid sequence of SEQ ID NO: 222.

52. The antibody of claim 51, wherein the VH comprises the amino acid sequence of SEQ ID NO: 219, and the VL comprises the amino acid sequence of SEQ ID NO: 223.

53. The antibody of claim 44 comprising:
the CDR-H1 comprises the amino acid sequence of SEQ ID NO: 217, the CDR-H2 comprises the amino acid sequence of SEQ ID NO: 218, the CDR-H3 comprises the amino acid sequence of SEQ ID NO: 67; and/or
the CDR-L1 comprises the amino acid sequence of SEQ ID NO: 224, the CDR-L2 comprises the amino acid sequence of SEQ ID NO: 69, and the CDR-L3 comprises the amino acid sequence of SEQ ID NO: 225.

54. The antibody of claim 53, wherein the VH comprises the amino acid sequence of SEQ ID NO: 219, and the VL comprises the amino acid sequence of SEQ ID NO: 226.

55. An antibody that binds to a SARS-CoV-2 spike protein, the antibody comprising:
a VH comprising a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3; and/or a VL comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
a VH comprising a CDR-H1 comprising the amino acid sequence of SEQ ID NO: 9, a CDR-H2 comprising the amino acid sequence of SEQ ID NO: 10, a CDR-H3 comprising the amino acid sequence of SEQ ID NO: 11; and/or a VL comprising a CDR-L1 comprising the amino acid sequence of SEQ ID NO: 12, a CDR-L2 comprising the amino acid sequence of SEQ ID NO: 13, and a CDR-L3 comprising the amino acid sequence of SEQ ID NO: 14;
a VH comprising a CDR-H1 comprising the amino acid sequence of SEQ ID NO: 17, a CDR-H2 comprising the amino acid sequence of SEQ ID NO: 18, a CDR-H3 comprising the amino acid sequence of SEQ ID NO: 19; and/or a VL comprising a CDR-L1 comprising the amino acid sequence of SEQ ID NO: 20, a CDR-L2 comprising the amino acid sequence of SEQ ID NO: 21, and a CDR-L3 comprising the amino acid sequence of SEQ ID NO: 22;
a VH comprising a CDR-H1 comprising the amino acid sequence of SEQ ID NO: 25, a CDR-H2 comprising the amino acid sequence of SEQ ID NO: 26, a CDR-H3 comprising the amino acid sequence of SEQ ID NO: 27; and/or a VL comprising a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
a VH comprising a CDR-H1 comprises the amino acid sequence of SEQ ID NO: 33, a CDR-H2 comprising the amino acid sequence of SEQ ID NO: 34, a CDR-H3 comprising the amino acid sequence of SEQ ID NO: 35; and/or a VL comprising a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 38;
a VH comprising a CDR-H1 comprising the amino acid sequence of SEQ ID NO: 49, a CDR-H2 comprising the amino acid sequence of SEQ ID NO: 50, a CDR-H3 comprising the amino acid sequence of SEQ ID NO: 51; and/or a VL comprising a CDR-L1 comprising the amino acid sequence of SEQ ID NO: 52, a CDR-L2 comprising the amino acid sequence of SEQ ID NO: 53 and a CDR-L3 comprising the amino acid sequence of SEQ ID NO: 54;
a VH comprising a CDR-H1 comprising the amino acid sequence of SEQ ID NO: 57, a CDR-H2 comprising the amino acid sequence of SEQ ID NO: 58, a CDR-H3 comprising the amino acid sequence of SEQ ID NO: 59; and/or a VL comprising a CDR-L1 comprising the amino acid sequence of SEQ ID NO: 60, a CDR-L2 comprising the amino acid sequence of SEQ ID NO: 61, and a CDR-L3 comprising the amino acid sequence of SEQ ID NO: 62;
a VH comprising a CDR-H1 comprising the amino acid sequence of SEQ ID NO: 73, a CDR-H2 comprising the amino acid sequence of SEQ ID NO: 74, a CDR-H3 comprising the amino acid sequence of SEQ ID NO: 75; and/or a VL comprising a CDR-L1 comprising the amino acid sequence of SEQ ID NO: 76, a CDR-L2 comprising the amino acid sequence of SEQ ID NO: 77, and a CDR-L3 comprising the amino acid sequence of SEQ ID NO: 78;
a VH comprising a CDR-H1 comprising the amino acid sequence of SEQ ID NO: 81, a CDR-H2 comprising the amino acid sequence of SEQ ID NO: 82, a CDR-H3 comprising the amino acid sequence of SEQ ID NO: 83; and/or a VL comprising a CDR-L1 comprising the amino acid sequence of SEQ ID NO: 84, a CDR-L2 comprising the amino acid sequence of SEQ ID NO: 37, and a CDR-L3 comprising the amino acid sequence of SEQ ID NO: 85;
a VH comprising a CDR-H1 comprising the amino acid sequence of SEQ ID NO: 88, a CDR-H2 comprising the amino acid sequence of SEQ ID NO: 89, a CDR-H3 comprising the amino acid sequence of SEQ ID NO: 90; and/or a VL comprising a CDR-L1 comprising the amino acid sequence of SEQ ID NO: 91, a CDR-L2 comprising the amino acid sequence of SEQ ID NO: 92, and a CDR-L3 comprising the amino acid sequence of SEQ ID NO: 93;
a VH comprising a CDR-H1 comprising the amino acid sequence of SEQ ID NO: 96, a CDR-H2 comprising the amino acid sequence of SEQ ID NO: 97, a CDR-H3 comprising the amino acid sequence of SEQ ID NO: 98; and/or a VL comprising a CDR-L1 comprising the amino acid sequence of SEQ ID NO: 99, a CDR-L2 comprising the amino acid sequence of SEQ ID NO: 100, and a CDR-L3 comprising the amino acid sequence of SEQ ID NO: 101;
a VH comprising a CDR-H1 comprising the amino acid sequence of SEQ ID NO: 104, a CDR-H2 comprising the amino acid sequence of SEQ ID NO: 105, a CDR-H3 comprising the amino acid sequence of SEQ ID NO: 106; and/or a VL comprising a CDR-L1 comprising the amino acid sequence of SEQ ID NO: 107, a CDR-L2 comprising the amino acid sequence of SEQ ID NO: 108, and a CDR-L3 comprising the amino acid sequence of SEQ ID NO: 109;
a VH comprising a CDR-H1 comprising the amino acid sequence of SEQ ID NO: 112, a CDR-H2 comprising the amino acid sequence of SEQ ID NO: 113, a CDR-H3 comprising the amino acid sequence of SEQ ID NO: 114; and/or a VL comprising a CDR-L1 comprising the amino acid sequence of SEQ ID NO: 115, a CDR-L2 comprising the amino acid sequence of SEQ ID NO: 45, and a CDR-L3 comprising the amino acid sequence of SEQ ID NO: 116;
a VH comprising a CDR-H1 comprising the amino acid sequence of SEQ ID NO: 127, a CDR-H2 comprising the amino acid sequence of SEQ ID NO: 128, a CDR-H3 comprising the amino acid sequence of SEQ ID NO: 129; and/or a VL comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 130; or
a VH comprising a CDR-H1 comprising the amino acid sequence of SEQ ID NO: 141, a CDR-H2 comprising the amino acid sequence of SEQ ID NO: 142, a CDR-H3 comprising the amino acid sequence of SEQ ID NO: 143; and/or a VL comprising a CDR-L1 comprising the amino acid sequence of SEQ ID NO: 144, a CDR-L2 comprising the amino acid sequence of SEQ ID NO: 145, and a CDR-L3 comprising the amino acid sequence of SEQ ID NO: 146.

56. The antibody of claim 55, wherein:
the CDR-H1 comprises the amino acid sequence of SEQ ID NO: 1, the CDR-H2 comprises the amino acid sequence of SEQ ID NO: 2, and the CDR-H3 comprises the amino acid sequence of SEQ ID NO: 3; and/or
the CDR-L1 comprises the amino acid sequence of SEQ ID NO: 4, the CDR-L2 comprises the amino acid sequence of SEQ ID NO: 5, and the CDR-L3 comprises the amino acid sequence of SEQ ID NO: 6.

57. The antibody of claim 56, wherein the VH comprises the amino acid sequence of SEQ ID NO: 7, and the VL comprises the amino acid sequence of SEQ ID NO: 8.

58. The antibody of claim 55, wherein:
the CDR-H1 comprises the amino acid sequence of SEQ ID NO: 9, the CDR-H2 comprises the amino acid sequence of SEQ ID NO: 10, and the CDR-H3 comprises the amino acid sequence of SEQ ID NO: 11; and/or
the CDR-L1 comprises the amino acid sequence of SEQ ID NO: 12, the CDR-L2 comprises the amino acid sequence of SEQ ID NO: 13, and the CDR-L3 comprises the amino acid sequence of SEQ ID NO: 14.

59. The antibody of claim 58, wherein the VH comprises the amino acid sequence of SEQ ID NO: 15, and the VL comprises the amino acid sequence of SEQ ID NO: 16.

60. The antibody of claim 55, wherein:
the CDR-H1 comprises the amino acid sequence of SEQ ID NO: 17, the CDR-H2 comprises the amino acid sequence of SEQ ID NO: 18, and the CDR-H3 comprises the amino acid sequence of SEQ ID NO: 19; and/or
the CDR-L1 comprises the amino acid sequence of SEQ ID NO: 20, the CDR-L2 comprises the amino acid sequence of SEQ ID NO: 21, and the CDR-L3 comprises the amino acid sequence of SEQ ID NO: 22.

61. The antibody of claim 60, wherein the VH comprises the amino acid sequence of SEQ ID NO: 23, and the VL comprises the amino acid sequence of SEQ ID NO: 24.

62. The antibody of claim 55, wherein:
the CDR-H1 comprises the amino acid sequence of SEQ ID NO: 25, the CDR-H2 comprises the amino acid sequence of SEQ ID NO: 26, and the CDR-H3 comprises the amino acid sequence of SEQ ID NO: 27; and/or
the CDR-L1 comprises the amino acid sequence of SEQ ID NO: 28, the CDR-L2 comprises the amino acid sequence of SEQ ID NO: 29, and the CDR-L3 comprises the amino acid sequence of SEQ ID NO: 30.

63. The antibody of claim 62, wherein the VH comprises the amino acid sequence of SEQ ID NO: 31, and the VL comprises the amino acid sequence of SEQ ID NO: 32.

64. The antibody of claim 55, wherein:
the CDR-H1 comprises the amino acid sequence of SEQ ID NO: 33, the CDR-H2 comprises the amino acid sequence of SEQ ID NO: 34, and the CDR-H3 comprises the amino acid sequence of SEQ ID NO: 35; and/or
the CDR-L1 comprises the amino acid sequence of SEQ ID NO: 36, the CDR-L2 comprises the amino acid sequence of SEQ ID NO: 37, and the CDR-L3 comprises the amino acid sequence of SEQ ID NO: 38.

65. The antibody of claim 64, wherein the VH comprises the amino acid sequence of SEQ ID NO: 39, and the VL comprises the amino acid sequence of SEQ ID NO: 40.

66. The antibody of claim 55, wherein:
the CDR-H1 comprises the amino acid sequence of SEQ ID NO: 49, the CDR-H2 comprises the amino acid sequence of SEQ ID NO: 50, and the CDR-H3 comprises the amino acid sequence of SEQ ID NO: 51; and/or
the CDR-L1 comprises the amino acid sequence of SEQ ID NO: 52, the CDR-L2 comprises the amino acid sequence of SEQ ID NO: 53 and the CDR-L3 comprises the amino acid sequence of SEQ ID NO: 54.

67. The antibody of claim 66, wherein the VH comprises the amino acid sequence of SEQ ID NO: 55, and the VL comprises the amino acid sequence of SEQ ID NO: 56.

68. The antibody of claim 55, wherein:
the CDR-H1 comprises the amino acid sequence of SEQ ID NO: 57, the CDR-H2 comprises the amino acid sequence of SEQ ID NO: 58, and the CDR-H3 comprises the amino acid sequence of SEQ ID NO: 59; and/or
the CDR-L1 comprises the amino acid sequence of SEQ ID NO: 60, the CDR-L2 comprises the amino acid sequence of SEQ ID NO: 61, and the CDR-L3 comprises the amino acid sequence of SEQ ID NO: 62.

69. The antibody of claim 68, wherein the VH comprises the amino acid sequence of SEQ ID NO: 63, and the VL comprises the amino acid sequence of SEQ ID NO: 64.

70. The antibody of claim 55, wherein:
the CDR-H1 comprises the amino acid sequence of SEQ ID NO: 73, the CDR-H2 comprises the amino acid sequence of SEQ ID NO: 74, and the CDR-H3 comprises the amino acid sequence of SEQ ID NO: 75; and/or
the CDR-L1 comprises the amino acid sequence of SEQ ID NO: 76, the CDR-L2 comprises the amino acid sequence of SEQ ID NO: 77, and the CDR-L3 comprises the amino acid sequence of SEQ ID NO: 78.

71. The antibody of claim 70, wherein the VH comprises the amino acid sequence of SEQ ID NO: 79, and the VL comprises the amino acid sequence of SEQ ID NO: 80.

72. The antibody of claim 55, wherein:
the CDR-H1 comprises the amino acid sequence of SEQ ID NO: 81, the CDR-H2 comprises the amino acid sequence of SEQ ID NO: 82, and the CDR-H3 comprises the amino acid sequence of SEQ ID NO: 83; and/or
the CDR-L1 comprises the amino acid sequence of SEQ ID NO: 84, the CDR-L2 comprises the amino acid sequence of SEQ ID NO: 37, and the CDR-L3 comprises the amino acid sequence of SEQ ID NO: 85.

73. The antibody of claim 72, wherein the VH comprises the amino acid sequence of SEQ ID NO: 86, and the VL comprises the amino acid sequence of SEQ ID NO: 87.

74. The antibody of claim 55, wherein:
the CDR-H1 comprises the amino acid sequence of SEQ ID NO: 88, the CDR-H2 comprises the amino acid sequence of SEQ ID NO: 89, and the CDR-H3 comprises the amino acid sequence of SEQ ID NO: 90; and/or
the CDR-L1 comprises the amino acid sequence of SEQ ID NO: 91, the CDR-L2 comprises the amino acid sequence of SEQ ID NO: 92, and the CDR-L3 comprises the amino acid sequence of SEQ ID NO: 93.

75. The antibody of claim 74, wherein the VH comprises the amino acid sequence of SEQ ID NO: 94, and the VL comprises the amino acid sequence of SEQ ID NO: 95.

76. The antibody of claim 55, wherein:
the CDR-H1 comprises the amino acid sequence of SEQ ID NO: 96, the CDR-H2 comprises the amino acid sequence of SEQ ID NO: 97, and the CDR-H3 comprises the amino acid sequence of SEQ ID NO: 98; and/or
the CDR-L1 comprises the amino acid sequence of SEQ ID NO: 99, the CDR-L2 comprises the amino acid sequence of SEQ ID NO: 100, and the CDR-L3 comprises the amino acid sequence of SEQ ID NO: 101.

77. The antibody of claim 76, wherein the VH comprises the amino acid sequence of SEQ ID NO: 102, and the VL comprises the amino acid sequence of SEQ ID NO: 103.

78. The antibody of claim 55, wherein:
the CDR-H1 comprises the amino acid sequence of SEQ ID NO: 104, the CDR-H2 comprises the amino acid sequence of SEQ ID NO: 105, and the CDR-H3 comprises the amino acid sequence of SEQ ID NO: 106; and/or
the CDR-L1 comprises the amino acid sequence of SEQ ID NO: 107, the CDR-L2 comprises the amino acid sequence of SEQ ID NO: 108, and the CDR-L3 comprises the amino acid sequence of SEQ ID NO: 109.

79. The antibody of claim 78, wherein the VH comprises the amino acid sequence of SEQ ID NO: 110, and the VL comprises the amino acid sequence of SEQ ID NO: 111.

80. The antibody of claim 55, wherein:
the CDR-H1 comprises the amino acid sequence of SEQ ID NO: 112, the CDR-H2 comprises the amino acid sequence of SEQ ID NO: 113, and the CDR-H3 comprises the amino acid sequence of SEQ ID NO: 114; and/or
the CDR-L1 comprises the amino acid sequence of SEQ ID NO: 115, the CDR-L2 comprises the amino acid sequence of SEQ ID NO: 45, and the CDR-L3 comprises the amino acid sequence of SEQ ID NO: 116.

81. The antibody of claim 80, wherein the VH comprises the amino acid sequence of SEQ ID NO: 117, and the VL comprises the amino acid sequence of SEQ ID NO: 118.

82. The antibody of claim 55, wherein:
the CDR-H1 comprises the amino acid sequence of SEQ ID NO: 127, the CDR-H2 comprises the amino acid sequence of SEQ ID NO: 128, and the CDR-H3 comprises the amino acid sequence of SEQ ID NO: 129; and/or
the CDR-L1 comprises the amino acid sequence of SEQ ID NO: 4, the CDR-L2 comprises the amino acid sequence of SEQ ID NO: 5, and the CDR-L3 comprises the amino acid sequence of SEQ ID NO: 130.

83. The antibody of claim 82, wherein the VH comprises the amino acid sequence of SEQ ID NO: 131, and the VL comprises the amino acid sequence of SEQ ID NO: 132.

84. The antibody of claim 55, wherein:
the CDR-H1 comprises the amino acid sequence of SEQ ID NO: 141, the CDR-H2 comprises the amino acid sequence of SEQ ID NO: 142, and the CDR-H3 comprises the amino acid sequence of SEQ ID NO: 143; and/or
the CDR-L1 comprises the amino acid sequence of SEQ ID NO: 144, the CDR-L2 comprises the amino acid sequence of SEQ ID NO: 145, and the CDR-L3 comprises the amino acid sequence of SEQ ID NO: 146.

85. The antibody of claim 84, wherein the VH comprises the amino acid sequence of SEQ ID NO: 147, and the VL comprises the amino acid sequence of SEQ ID NO: 148.

86. The antibody of any one of claims 1-85, wherein the antibody is selected from the group consisting of a full length IgG, a Fab fragment, a F(ab′) fragment, a F(ab′)2 fragment, an scFv, and Fv.

87. The antibody of claim 86, wherein the antibody is a full length lgG.

88. The antibody of claim 87, wherein the full length IgG comprises a heavy chain constant region of isotype IgG1, IgG2, IgG3, or IgG4.

89. The antibody of claim 86, wherein the antibody is a Fab.

90. The antibody of any one of claims 1-89, wherein the antibody binds to a SARS-CoV-2 spike protein of SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, or SARS-CoV-2 Omicron.

91. The antibody of any one of claims 1-90, wherein the antibody binds to a SARS-COV-2 spike protein with a K_{D} between 1.0×10⁻⁸ M and 1.0×10⁻¹⁰ M.

92. The antibody of any one of claims 1-91, wherein the antibody is a humanized antibody.

93. The antibody of any one of claims 1-91, wherein the antibody is a human antibody.

94. A pharmaceutical composition comprising the antibody of any one of claims 1-93 and a pharmaceutically acceptable excipient.

95. A nucleic acid sequence encoding the antibody of any one of claims 1-93.

96. A vector comprising the nucleic acid sequence of claim 95.

97. A cell comprising the antibody of any one of claims 1-93, the nucleic acid sequence of claim 95, or the vector of claim 96.

98. A method of treating a subject infected with a coronavirus, the method comprising administering the antibody of any one of claims 1-93, the pharmaceutical composition of claim 94, the nucleic acid sequence of claim 95, the vector of claim 96, or the cell of claim 97 to the subject.

99. The method of claim 98, wherein the subject is infected with severe acute respiratory syndrome corona virus 1 (SARS-CoV-1), SARS-CoV-2 or Bat coronavirus WIV16.

100. The method of claim 98, wherein the subject is infected with SARS-CoV-2 alpha, SARS-CoV-2 beta, SARS-CoV-2 Delta, SARS-CoV-2 Epsilon, SARS-CoV-2 Gamma, SARS-CoV-2 Kappa, SARS-CoV-2 Iota, or SARS-CoV-2 Omicron.

101. The method of any one of claims 98-100, wherein the subject is a human subject.

102. The method of any one of claims 98-101, wherein the subject has an underlying condition that increases the risk of severe disease or death from SARS-CoV-2 infection.

103. The method of claim 102, wherein the underlying condition is cancer, chronic liver disease, chronic kidney disease, chronic lung disease, cystic fibrosis, dementia, diabetes, disability, heart disease, HIV infection, immunocompromise, obesity, pregnancy, sickle cell disease, thalassemia, smoking, transplant recipient, cerebrovascular disease, substance abuse disorders, or tuberculosis.

104. The method of any one of claims 98-103, further comprising administering an additional therapeutic for treatment of coronavirus.

105. The method of claim 104, wherein the additional therapeutic comprises an antiviral molecule or an antibody.

106. The method of claim 105, wherein the antiviral molecule is nirmatrelvir and ritonavir, remdesivir, or molnupiravir.
